SEQUENCE LISTING (58 sequences)

SEQ ID NO: 1 - DNA, 5804 bp, homo sapien
aaaaggtctgtcatcctccagttgaagtctgtgtgcctaccaagaatggccggaggactcgctgcttgacgggagggatgctccagcttggtctccagggacaactgtacctggagataaagtctggataccagggagggacagagatgctcttccttccacagtgtggccacttgctgggccatgtgaaccagcagaggagagttccttggctatgctgttgttcccccgctgtccagggagaatggaggtggactgaggagtgaagtttgggcgaactgcacagagctgctcctcttcaccccgaaaatttgtctttttacagaatccaggttctcccctccctcatcactgctgttgcttcctttgaaagtctaaaccactggaggcttctttttccttcctctctccttccccagtttcctctgtcccaattaaaaccaggatggagagcatttgctggctggccccaattattgtacccttgcccaaagggaggggcctctgtcgtgacccctcagaaatctggcagtctgggtttccacctttcccctatttaactctcagcccccacccatcccctggggttgcggctggcaggccgggacttgcagaaccaaatgggccaggggccaagttcattcttttgggagagagcaatggtaccttcccttaacgggaaaacgagaaatattgaggggaagatggactgcgatccaaacgccctggctctcaggcctggactctagggcttagccagatgcctaaaccgcccaagccgagaaacaacttagaagacagacataaccctgggattcagggaaggcgcgagcaccgcccaggacctggtagggtgcgagccgcgagcagtccgggagggagcgcgcctagggcggagcgtaggctgtggggggagggctgggagtccggggccgccccacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccaggtaaggctggagctccgaactgactaaggcggggccagccacagaagggggcgtgtcggagaccacagaaaggcttggtctcatccctgggcctgggaagaagccgactatcccttggggttcgaaagtgggtggagataggagtctccgggtttgagaagggatgaggctgaatctttggtgccccaaggatccccaaggagtcgagttccaggcctcagacccaagatggaatgtccatacccttcaccctcatccaagagcagtgtgggctgggatagggaggggacatgggtcataggctcttctaggtagagccagggccccggcgttgtgctttcccacccttctagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggtgagggccgggcaccatacttagctccccgaaaccaaggcagaggaaggcaggggtctgctcccagaccactccttcaaagcccccagagaggatgctgatttcattggcacatcctttttctccccatcatttcactccccagtctcctcacctcctactcatttctccacctcccccagaccacccatatttccaggacagcctggtctcggtcctcagcttctgtcctgcctccgcctctttctgtgggaaacccagcgggggtgcaaacagataggtagagcttggagtttgcccttcccctgatcgtcccataactgccaccccacaggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggtcagtctccccaaccccaccttcattaattcatccagtagacatttattaagcacgtactatggtgtgagtcctccagggtagataatctagaggacggatggggcagagccctactcaccaggagctctgtacaatagggcaatgtcacctgcctagacctacagactgaggaacggtgaggggggccatacagccacataactccaggctgcagggtcaaaggcagggaaggactgtgtcagtccaggaacaggcaagtaagagaagagggagagagagacaaggaagtgagagtgtggaggatgagatctttggggctgggaagtagggaaagggcattcacctcttctaaggctggggacaggggccgggggaggggaaagagggccagcacggcttcctgacggtgtgactcccacagtgggcccttcccagggaaagactacagtgatggctggggtctggaaagaggggcttgggctccccactctgctcctaatcttacctcccttcttcccccctgcccacttctggcaggggccacagtggtgctgaaatccaagttctcggctggtcagttctgggaagattgccagcagcacagggtgacggtgttccagtacattggggagctgtgccgataccttgtcaaccagcccccggtgcgtgggcacagatcctgggcagagctgctgacacagggctagctcacggggagcaggacagttgacaggagacagggagttggagggagaagcagcaagaagaaaatggcagtgtaagataaggagccagagatggcctaggccatctgatgcttacggcctctgaaggaggtcacagtaggagggggttctggggaatggggaacaggaattcacttccgggagcgggcatcttgatgctgaagctccggcccctctcccactcatctcagagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaaggtgaggggcagagaggaaactgaaaacccgtggaacagcagagggctggcaggagagggggctcatgtgactgcaatgatccagtacccaggtctccctttccccagcatatcttccccttctccttgattcgctatgatgtcaccacaggagagccaattcgggacccccaggggcactgtatggccacatctccaggttggtggtgttctggtggggtgggcggggtgctgaagctggcacaggaggactggaattggagactggggtggatgggggcagaaggctctgggaaaggtgacaccactcctgaccctggtgactctgccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtatctgtccataactggtttttcatcctggacatctgatctctgtgatccaaagcttctgaacctcaactctctaatctgccacctcaacctgggtcctaagctaatctctcattctcagatctcaccatttcatccctgtgacactgacctctgacctcatctccccaccaagcccataaggccctgacccctgactcccagtttcagatctctgctctctgacaggtggaagggggagaatgtggccacaaccgaggtggcagaggtcttcgaggccctagattttcttcaggaggtgaacgtctatggagtcactgtgccaggtgcctaggcatggaaggtgggggaggcacccagccaccaccccgaattggtagtacttgggcgcagggagcacgaggccttcgtggtggtcagccatggaggggcttactctgtctcccacacccaccagggcatgaaggcagggctggaatggcagccctagttctgcgtcccccccacgctttggaccttatgcagctctacacccacgtgtctgagaacttgccaccttatgcccggccccgattcctcaggctccaggtaaccggccacttcccccgccggcccctcaccccatatatcctcaccccacatatccaccccgtgtatcaacttaggagtttgatggctcccaaactccacaaagggacccccaacgtaatacactccgtgaagagaaaaaacacggagacacaagctcttcaccccacttctttccttatccctgccttcttctggcctggctctttctcctccctgtcacctcctcccctaaaccttgacctcacactccctttcccaagacttgctccttaacaaaattcaggcaactcccctgaaccacgtgggcagagtccccttccctccaaatccctgaccctcctgtccccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctaca

SEQ ID NO: 2 - RNA, 3351 nt, homo sapien
aaaaggucugucauccuccaguugaagucugugugccuaccaagaauggccggaggacucgcugcuugacgggagggaugcuccagcuuggucuccagggacaacuguaccuggagauaaagucuggauaccagggagggacagagaugcucuuccuuccacaguguggccacuugcugggccaugugaaccagcagaggagaguuccuuggcuaugcuguuguucccccgcuguccagggagaauggagguggacugaggagugaaguuugggcgaacugcacagagcugcuccucuucaccccgaaaauuugucuuuuuacagaauccagguucuccccucccucaucacugcuguugcuuccuuugaaagucuaaaccacuggaggcuucuuuuuccuuccucucuccuuccccaguuuccucugucccaauuaaaaccaggauggagagcauuugcuggcuggccccaauuauuguacccuugcccaaagggaggggccucugucgugaccccucagaaaucuggcagucuggguuuccaccuuuccccuauuuaacucucagcccccacccauccccugggguugcggcuggcaggccgggacuugcagaaccaaaugggccaggggccaaguucauucuuuugggagagagcaaugguaccuucccuuaacgggaaaacgagaaauauugaggggaagauggacugcgauccaaacgcccuggcucucaggccuggacucuagggcuuagccagaugccuaaaccgcccaagccgagaaacaacuuagaagacagacauaacccugggauucagggaaggcgcgagcaccgcccaggaccugguagggugcgagccgcgagcaguccgggagggagcgcgccuagggcggagcguaggcuguggggggagggcugggaguccggggccgccccacacccgcacuccucccggguuucugcucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuaca

SEQ ID NO: 3 - RNA, 2298 nt, homo sapien
agaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuaca

SEQ ID NO: 4 - RNA, 2193 nt, homo sapien
agaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuaca

SEQ ID NO: 5 - RNA, 3343 nt, homo sapien
aaaaggucugucauccuccaguugaagucugugugccuaccaagaauggccggaggacucgcugcuugacgggagggaugcuccagcuuggucuccagggacaacuguaccuggggauaaagucuggauaccagggagggacagagaugcucuuccuuccacaguguggccacuugcugggccaugugaaccagcagaggagaguuccuuggcuaugcuguuguucccccgcuguccagggagaauggagguggacugaggagugaaguuugggcgaacugcacagagcugcuccucuucaccccgaaaauuugucuuuuuacagaauccagguucuccccucccucaucacugcuguugcuuccuuugaaagucuaaaccacuggaggcuucuuuuuccuuccucucuccuuccccaguuuccucugucccaauuaaaaccaggauggagagcauuugcuggcuggccccaauuauuguacccuugcccaaagggaggggccucugucgugaccccucagaaaucuggcagucuggguuuccaccuuuccccuauuuaacucucagcccccacccauccccugggguugcggcuggcaggccgggacuugcagaaccaaaugggccaggggccaaguucauucuuuugggagagagcaaugguaccuucccuuaacgggaaaacgagaaauauugaggggaagauggacugcgauccaaacgcccuggcucucaggccuggacucuagggcuuagccagaugccuaaaccgcccaagccgagaaacaacuuagaagacagauauaacccugggauucagggaaggcgcgagcaccgcccaggaccugguagggugcgagccgcgagcaguccgggagggagcgcgccuagggcggagcguaggcuguggggggagggcugggaguccggggccgccccacacccgcacuccucccggguuucugcucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucug

SEQ ID NO: 6 - RNA, 2430 nt, homo sapien
cacacccgcacuccucccggguuucugcucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuacaaaaaaaaaaaaaaaaaa

SEQ ID NO: 7 - RNA, 2405 nt, homo sapien
gcacuccucccggguuucugcucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuac

SEQ ID NO: 8 - RNA, 2414 nt, homo sapien
cccacgcguccgcugcucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuaaaaaaaaaaaaaaaa

SEQ ID NO: 9 - RNA, 4507 nt, homo sapien
cucuccgcccguguggaguggugggggccugggugggaaugggcgugugccagcgcacgcgcgcucccuggaaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagguaaggcuggagcuccgaacugacuaaggcggggccagccacagaagggggcgugucggagaccacagaaaggcuuggucucaucccugggccugggaagaagccgacuaucccuugggguucgaaaguggguggagauaggagucuccggguuugagaagggaugaggcugaaucuuuggugccccaaggauccccaaggagucgaguuccaggccucagacccaagauggaauguccauacccuucacccucauccaagagcagugugggcugggauagggaggggacaugggucauaggcucuucuagguagagccagggccccggcguugugcuuucccacccuucuagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggucagucuccccaaccccaccuucauuaauucauccaguagacauuuauuaagcacguacuauggugugaguccuccaggguagauaaucuagaggacggauggggcagagcccuacucaccaggagcucuguacaauagggcaaugucaccugccuagaccuacagacugaggaacggugaggggggccauacagccacauaacuccaggcugcagggucaaaggcagggaaggacugugucaguccaggaacaggcaaguaagagaagagggagagagagacaaggaagugagaguguggaggaugagaucuuuggggcugggaaguagggaaagggcauucaccucuucuaaggcuggggacaggggccgggggaggggaaagagggcccagcacggcuuccugacggugugacucccacagugggcccuucccagggaaagacuacagugauggcuggggucuggaaagaggggcuugggcuccccacucugcuccuaaucuuaccuccuuccuucuucccccugcccacuucuggcaggggccacaguggugcugaaauccaaguucucggcuggucaguucugggaagauugccagcagcacagggugacgguguuccaguacauuggggagcugugccgauaccuugucaaccagcccccggugcgugggcacagauccugggcagagcugcugacacagggcuagcucacggggagcaggacaguugacaggagacagggaguuggagggagaagcagcaagaagaaaauggcaguguaagauaaggagccagagauggccuaggccaucugaugcuuacggccucugaaggaggucacaguaggaggggguucuggggaauggggaacaggaauucacuuccgggagcgggcaucuugaugcugaagcuccggccccucucccacucaucucagagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaaggugaggggcagagaggaaacugaaaacccguggaacagcagagggcuggcaggagagggggcucaugugacugcaaugauccaguacccaggucucccuuuccccagcauaucuuccccuucuccuugauucgcuaugaugucaccacaggagagccaauucgggacccccaggggcacuguauggccacaucuccagguuggugguguucugguggggugggcggggugcugaagcuggcacaggaggacuggaauuggagacugggguggaugggggcagaaggcucugggaaaggugacaccacuccugacccuggugacucugccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguaucuguccauaacugguuuuucauccuggacaucugaucucugugauccaaagcuucugaaccucaacucucuaaucugccaccucaaccuggguccuaagcuaaucucucauucucagaucucaccauuucaucccugugacacugaccucugaccucaucuccccaccaagcccauaaggcccugaccccugacucccaguuucagaucucugcucucugacagguggaagggggagaauguggccacaaccgagguggcagaggucuucgaggcccuagauuuucuucaggaggugaacgucuauggagucacugugccaggugccuaggcauggaaggugggggaggcacccagccaccaccccgaauugguaguacuugggcgcagggagcacgaggccuucgugguggucagccauggaggggcuuacucugucucccacacccaccagggcaugaaggcagggcuggaauggcagcccuaguucugcgucccccccacgcuuuggaccuuaugcagcucuacacccacgugucugagaacuugccaccuuaugcccggccccgauuccucaggcuccagguaaccggccacuucccccgccggccccucaccccauauauccucaccccacauauccaccccguguaucaacuuaggaguuugauggcucccaaacuccacaaagggacccccagcguaauacacuccgugaagagaaaaaacacggagacacaagcucuucaccccacuucuuuccuuaucccugcccucuucuggccuggcucuuucuccucccugucaccuccuccccuaaaccuugaccucacacucccuuucccaagacuugcuccuuaacaaaauucaggcaacuccccugaaccacgugggcagaguccccuucccuccaaaucccugacccuccuguccccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuac

SEQ ID NO: 10 - RNA, 2314 nt, homo sapien
gggaggagaagucucagcuagaacgagcggcccuagguuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagaguauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccu

SEQ ID NO: 11 - RNA, 2333 nt, homo sapien
guuuucggaagggaggaucagggauguuugcgagcggcuggaaccagacggugccgauagaggaagcgggcuccauggcugcccuccugcugcugccccugcugcuguugcuaccgcugcugcugcugaagcuacaccucuggccgcaguugcgcuggcuuccggcggacuuggccuuugcggugcgagcucugugcugcaaaagggcucuucgagcucgcgcccuggccgcggcugccgccgacccggaaggucccgaggggggcugcagccuggccuggcgccucgcggaacuggcccagcagcgcgccgcgcacaccuuucucauucacggcucgcggcgcuuuagcuacucagaggcggagcgcgagaguaacagggcugcacgcgccuuccuacgugcgcuaggcugggacuggggacccgacggcggcgacagcggcgaggggagcgcuggagaaggcgagcgggcagcgccgggagccggagaugcagcggccggaagcggcgcggaguuugccggaggggacggugccgccagagguggaggagccgccgccccucugucaccuggagcaacuguggcgcugcuccuccccgcuggcccagaguuucuguggcucugguucgggcuggccaaggccggccugcgcacugccuuugugcccaccgcccugcgccggggcccccugcugcacugccuccgcagcugcggcgcgcgcgcgcuggugcuggcgccagaguuucuggagucccuggagccggaccugcccgcccugagagccauggggcuccaccugugggcugcaggcccaggaacccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagggcuucgaccccagcacccugucugacccacuguacguucuggaccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuacaaaaaaaaaaaaaaaaaaaaaaaaaaaaaaaaaaaaaaaaaaaaaaaaaaaaa

SEQ ID NO: 12 - RNA, 1380 nt, homo sapien
cccacccugcuggaauuagcgauuugcuggcugaaguguccgcugaaguggaugggccagugccaggauaccucucuuccccccagagcauaacagacacgugccuguacaucuucaccucuggcaccacgggccuccccaaggcugcucggaucagucaucugaagauccugcaaugccagggcuucuaucagcugugugguguccaccaggaagaugugaucuaccucgcccucccacucuaccacauguccgguucccugcugggcaucgugggcugcaugggcauuggggccacaguggugcugaaauccaaguucucggcuggucaguucugggaagauugccagcagcacagggugacgguguuccaguacauuggggagcugugccgauaccuugucaaccagcccccgagcaaggcagaacguggccauaagguccggcuggcagugggcagcgggcugcgcccagauaccugggagcguuuugugcggcgcuucgggccccugcaggugcuggagacauuuggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccaggugagccagggcugcugguggccccgguaagccagcaguccccauuccugggcuaugcuggcgggccagagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggcuguaggugccuaccugccccucacaacugcccgguacagcgcccuccuggcaggaaaccuucgaaucugagaacuuccacaccugaggcaccugagagaggaacucuguggggugggggccguugcagguguacugggcugucagggaucuuuucuauaccagaacugcggucacuauuuuguaauaaauguggcuggagcugauccagcugucucugaccuacaaaaaaaaaaaaaaaaaaaaaaaa

SEQ ID NO: 13 - RNA, 457 nt, homo sapien
caauucgggacccccaggggcacuguauggccacaucuccagcuggcccaggggaaguugcuaaaggaugucuuccggccuggggauguuuucuucaacacuggggaccugcuggucugcgaugaccaagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccccccacgcuuuggaccuuaugcagcucuacacccacgugucugagaacuugccaccuuaugcccggccccgauuccucaggcuccaggagucuuuggccaccacagagaccuucaaacagcagaaaguucggauggcaaaugagg

SEQ ID NO: 14 - RNA, 433 nt, homo sapien
gugggcagcgggcugcgcccagauaccugggagcguuuugugcggcgcuucgggccccugcaggugcuggagacauauggacugacagagggcaacguggccaccaucaacuacacaggacagcggggcgcuguggggcgugcuuccuggcuuuacaagcauaucuuccccuucuccuugauucgcuaugaugucaccacaggagagccaauucgggacccccaggggcacuguauggccacaucuccagguuuucuccgcuuccaugaucguacuggagacaccuucagguggaagggggagaauguggccacaaccgagguggcagaggucuucgaggcccuagauuuucuucaggaggugaacgucuauggagucacugugccagggcaugaaggcagggcuggaauggcagcccuaguucugcgucccc

SEQ ID NO: 15 - DNA, 3351 bp, homo sapien
aaaaggtctgtcatcctccagttgaagtctgtgtgcctaccaagaatggccggaggactcgctgcttgacgggagggatgctccagcttggtctccagggacaactgtacctggagataaagtctggataccagggagggacagagatgctcttccttccacagtgtggccacttgctgggccatgtgaaccagcagaggagagttccttggctatgctgttgttcccccgctgtccagggagaatggaggtggactgaggagtgaagtttgggcgaactgcacagagctgctcctcttcaccccgaaaatttgtctttttacagaatccaggttctcccctccctcatcactgctgttgcttcctttgaaagtctaaaccactggaggcttctttttccttcctctctccttccccagtttcctctgtcccaattaaaaccaggatggagagcatttgctggctggccccaattattgtacccttgcccaaagggaggggcctctgtcgtgacccctcagaaatctggcagtctgggtttccacctttcccctatttaactctcagcccccacccatcccctggggttgcggctggcaggccgggacttgcagaaccaaatgggccaggggccaagttcattcttttgggagagagcaatggtaccttcccttaacgggaaaacgagaaatattgaggggaagatggactgcgatccaaacgccctggctctcaggcctggactctagggcttagccagatgcctaaaccgcccaagccgagaaacaacttagaagacagacataaccctgggattcagggaaggcgcgagcaccgcccaggacctggtagggtgcgagccgcgagcagtccgggagggagcgcgcctagggcggagcgtaggctgtggggggagggctgggagtccggggccgccccacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctaca

SEQ ID NO: 16 - DNA, 2298 bp, homo sapien
agaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctaca

SEQ ID NO: 17 - DNA, 2193 bp, homo sapien
agaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctaca

SEQ ID NO: 18 - DNA, 3343 bp, homo sapien
aaaaggtctgtcatcctccagttgaagtctgtgtgcctaccaagaatggccggaggactcgctgcttgacgggagggatgctccagcttggtctccagggacaactgtacctggggataaagtctggataccagggagggacagagatgctcttccttccacagtgtggccacttgctgggccatgtgaaccagcagaggagagttccttggctatgctgttgttcccccgctgtccagggagaatggaggtggactgaggagtgaagtttgggcgaactgcacagagctgctcctcttcaccccgaaaatttgtctttttacagaatccaggttctcccctccctcatcactgctgttgcttcctttgaaagtctaaaccactggaggcttctttttccttcctctctccttccccagtttcctctgtcccaattaaaaccaggatggagagcatttgctggctggccccaattattgtacccttgcccaaagggaggggcctctgtcgtgacccctcagaaatctggcagtctgggtttccacctttcccctatttaactctcagcccccacccatcccctggggttgcggctggcaggccgggacttgcagaaccaaatgggccaggggccaagttcattcttttgggagagagcaatggtaccttcccttaacgggaaaacgagaaatattgaggggaagatggactgcgatccaaacgccctggctctcaggcctggactctagggcttagccagatgcctaaaccgcccaagccgagaaacaacttagaagacagatataaccctgggattcagggaaggcgcgagcaccgcccaggacctggtagggtgcgagccgcgagcagtccgggagggagcgcgcctagggcggagcgtaggctgtggggggagggctgggagtccggggccgccccacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctg

SEQ ID NO: 19 - DNA, 2430 bp, homo sapien
cacacccgcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctacaaaaaaaaaaaaaaaaaa

SEQ ID NO: 20 - DNA, 2405 bp, homo sapien
gcactcctcccgggtttctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctac

SEQ ID NO: 21 - DNA, 2414 bp, homo sapien
cccacgcgtccgctgctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctaaaaaaaaaaaaaaaa

SEQ ID NO: 22 - DNA, 4507 bp, homo sapien
ctctccgcccgtgtggagtggtgggggcctgggtgggaatgggcgtgtgccagcgcacgcgcgctccctggaaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccaggtaaggctggagctccgaactgactaaggcggggccagccacagaagggggcgtgtcggagaccacagaaaggcttggtctcatccctgggcctgggaagaagccgactatcccttggggttcgaaagtgggtggagataggagtctccgggtttgagaagggatgaggctgaatctttggtgccccaaggatccccaaggagtcgagttccaggcctcagacccaagatggaatgtccatacccttcaccctcatccaagagcagtgtgggctgggatagggaggggacatgggtcataggctcttctaggtagagccagggccccggcgttgtgctttcccacccttctagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggtcagtctccccaaccccaccttcattaattcatccagtagacatttattaagcacgtactatggtgtgagtcctccagggtagataatctagaggacggatggggcagagccctactcaccaggagctctgtacaatagggcaatgtcacctgcctagacctacagactgaggaacggtgaggggggccatacagccacataactccaggctgcagggtcaaaggcagggaaggactgtgtcagtccaggaacaggcaagtaagagaagagggagagagagacaaggaagtgagagtgtggaggatgagatctttggggctgggaagtagggaaagggcattcacctcttctaaggctggggacaggggccgggggaggggaaagagggcccagcacggcttcctgacggtgtgactcccacagtgggcccttcccagggaaagactacagtgatggctggggtctggaaagaggggcttgggctccccactctgctcctaatcttacctccttccttcttccccctgcccacttctggcaggggccacagtggtgctgaaatccaagttctcggctggtcagttctgggaagattgccagcagcacagggtgacggtgttccagtacattggggagctgtgccgataccttgtcaaccagcccccggtgcgtgggcacagatcctgggcagagctgctgacacagggctagctcacggggagcaggacagttgacaggagacagggagttggagggagaagcagcaagaagaaaatggcagtgtaagataaggagccagagatggcctaggccatctgatgcttacggcctctgaaggaggtcacagtaggagggggttctggggaatggggaacaggaattcacttccgggagcgggcatcttgatgctgaagctccggcccctctcccactcatctcagagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaaggtgaggggcagagaggaaactgaaaacccgtggaacagcagagggctggcaggagagggggctcatgtgactgcaatgatccagtacccaggtctccctttccccagcatatcttccccttctccttgattcgctatgatgtcaccacaggagagccaattcgggacccccaggggcactgtatggccacatctccaggttggtggtgttctggtggggtgggcggggtgctgaagctggcacaggaggactggaattggagactggggtggatgggggcagaaggctctgggaaaggtgacaccactcctgaccctggtgactctgccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtatctgtccataactggtttttcatcctggacatctgatctctgtgatccaaagcttctgaacctcaactctctaatctgccacctcaacctgggtcctaagctaatctctcattctcagatctcaccatttcatccctgtgacactgacctctgacctcatctccccaccaagcccataaggccctgacccctgactcccagtttcagatctctgctctctgacaggtggaagggggagaatgtggccacaaccgaggtggcagaggtcttcgaggccctagattttcttcaggaggtgaacgtctatggagtcactgtgccaggtgcctaggcatggaaggtgggggaggcacccagccaccaccccgaattggtagtacttgggcgcagggagcacgaggccttcgtggtggtcagccatggaggggcttactctgtctcccacacccaccagggcatgaaggcagggctggaatggcagccctagttctgcgtcccccccacgctttggaccttatgcagctctacacccacgtgtctgagaacttgccaccttatgcccggccccgattcctcaggctccaggtaaccggccacttcccccgccggcccctcaccccatatatcctcaccccacatatccaccccgtgtatcaacttaggagtttgatggctcccaaactccacaaagggacccccagcgtaatacactccgtgaagagaaaaaacacggagacacaagctcttcaccccacttctttccttatccctgccctcttctggcctggctctttctcctccctgtcacctcctcccctaaaccttgacctcacactccctttcccaagacttgctccttaacaaaattcaggcaactcccctgaaccacgtgggcagagtccccttccctccaaatccctgaccctcctgtccccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctac

SEQ ID NO: 23 - DNA, 2314 bp, homo sapien
gggaggagaagtctcagctagaacgagcggccctaggttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagtataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacct

SEQ ID NO: 24 - DNA, 2333 bp, homo sapien
gttttcggaagggaggatcagggatgtttgcgagcggctggaaccagacggtgccgatagaggaagcgggctccatggctgccctcctgctgctgcccctgctgctgttgctaccgctgctgctgctgaagctacacctctggccgcagttgcgctggcttccggcggacttggcctttgcggtgcgagctctgtgctgcaaaagggctcttcgagctcgcgccctggccgcggctgccgccgacccggaaggtcccgaggggggctgcagcctggcctggcgcctcgcggaactggcccagcagcgcgccgcgcacacctttctcattcacggctcgcggcgctttagctactcagaggcggagcgcgagagtaacagggctgcacgcgccttcctacgtgcgctaggctgggactggggacccgacggcggcgacagcggcgaggggagcgctggagaaggcgagcgggcagcgccgggagccggagatgcagcggccggaagcggcgcggagtttgccggaggggacggtgccgccagaggtggaggagccgccgcccctctgtcacctggagcaactgtggcgctgctcctccccgctggcccagagtttctgtggctctggttcgggctggccaaggccggcctgcgcactgcctttgtgcccaccgccctgcgccggggccccctgctgcactgcctccgcagctgcggcgcgcgcgcgctggtgctggcgccagagtttctggagtccctggagccggacctgcccgccctgagagccatggggctccacctgtgggctgcaggcccaggaacccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagggcttcgaccccagcaccctgtctgacccactgtacgttctggaccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctacaaaaaaaaaaaaaaaaaaaaaaaaaaaaaaaaaaaaaaaaaaaaaaaaaaaaa

SEQ ID NO: 25 - DNA, 1380 bp, homo sapien
cccaccctgctggaattagcgatttgctggctgaagtgtccgctgaagtggatgggccagtgccaggatacctctcttccccccagagcataacagacacgtgcctgtacatcttcacctctggcaccacgggcctccccaaggctgctcggatcagtcatctgaagatcctgcaatgccagggcttctatcagctgtgtggtgtccaccaggaagatgtgatctacctcgccctcccactctaccacatgtccggttccctgctgggcatcgtgggctgcatgggcattggggccacagtggtgctgaaatccaagttctcggctggtcagttctgggaagattgccagcagcacagggtgacggtgttccagtacattggggagctgtgccgataccttgtcaaccagcccccgagcaaggcagaacgtggccataaggtccggctggcagtgggcagcgggctgcgcccagatacctgggagcgttttgtgcggcgcttcgggcccctgcaggtgctggagacatttggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggtgagccagggctgctggtggccccggtaagccagcagtccccattcctgggctatgctggcgggccagagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggctgtaggtgcctacctgcccctcacaactgcccggtacagcgccctcctggcaggaaaccttcgaatctgagaacttccacacctgaggcacctgagagaggaactctgtggggtgggggccgttgcaggtgtactgggctgtcagggatcttttctataccagaactgcggtcactattttgtaataaatgtggctggagctgatccagctgtctctgacctacaaaaaaaaaaaaaaaaaaaaaaaa

SEQ ID NO: 26 - DNA, 457 bp, homo sapien
caattcgggacccccaggggcactgtatggccacatctccagctggcccaggggaagttgctaaaggatgtcttccggcctggggatgttttcttcaacactggggacctgctggtctgcgatgaccaaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccccccacgctttggaccttatgcagctctacacccacgtgtctgagaacttgccaccttatgcccggccccgattcctcaggctccaggagtctttggccaccacagagaccttcaaacagcagaaagttcggatggcaaatgagg

SEQ ID NO: 27 - DNA, 433 bp, homo sapien
gtgggcagcgggctgcgcccagatacctgggagcgttttgtgcggcgcttcgggcccctgcaggtgctggagacatatggactgacagagggcaacgtggccaccatcaactacacaggacagcggggcgctgtggggcgtgcttcctggctttacaagcatatcttccccttctccttgattcgctatgatgtcaccacaggagagccaattcgggacccccaggggcactgtatggccacatctccaggttttctccgcttccatgatcgtactggagacaccttcaggtggaagggggagaatgtggccacaaccgaggtggcagaggtcttcgaggccctagattttcttcaggaggtgaacgtctatggagtcactgtgccagggcatgaaggcagggctggaatggcagccctagttctgcgtcccc

SEQ ID NO: 28 - protein, 811 aa, homo sapien
MPKPPKPRNNLEDRHNPGIQGRREHRPGPGRVRAASSPGGSAPRAERRLWGEGWESGAAPHPHSSRVSALRPCGVVGAWVGMGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANEGFDPSTLSDPLYVLDQAVGAYLPLTTARYSALLAGNLRI

SEQ ID NO: 29 - protein, 683 aa, homo sapien
MAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANEGFDPSTLSDPLYVLDQAVGAYLPLTTARYSALLAGNLRI

SEQ ID NO: 30 - protein, 648 aa, homo sapien
MAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQAVGAYLPLTTARYSALLAGNLRI

SEQ ID NO: 31 - protein, 640 aa, homo sapien
MPKPPKPRNNLEDRYNPGIQGRREHRPGPGRVRAASSPGGSAPRAERRLWGEGWESGAAPHPHSSRVSALRPCGVVGAWVGMGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVASSPHSWAMLAGQSWPRGSC

SEQ ID NO: 32 - protein, 730 aa, homo sapien
MGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPEFLESLEPDLPALRAMGLHLWAAGPGTHPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETYGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANEGFDPSTLSDPLYVLDQAVGAYLPLTTARYSALLAGNLRI

SEQ ID NO: 33 - protein, 288 aa, homo sapien
LRPCGVVGAWVGMGVCQRTRAPWKEKSQLERAALGFRKGGSGMFASGWNQTVPIEEAGSMAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPGKAGAPN

SEQ ID NO: 34 - protein, 700 aa, homo sapien [sequence omitted]

SEQ ID NO: 35 - protein, 399 aa, homo sapien
HPAGISDLLAEVSAEVDGPVPGYLSSPQSITDTCLYIFTSGTTGLPKAARISHLKILQCQGFYQLCGVHQEDVIYLALPLYHMSGSLLGIVGCMGIGATVVLKSKFSAGQFWEDCQQHRVTVFQYIGELCRYLVNQPPSKAERGHKVRLAVGSGLRPDTWERFVRRFGPLQVLETFGLTEGNVATINYTGQRGAVGRASWLYKHIFPFSLIRYDVTTGEPIRDPQGHCMATSPGEPGLLVAPVSQQSPFLGYAGGPELAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQAVGAYLPLTTARYSALLAGNLRI

SEQ ID NO: 36 - protein, 152 aa, homo sapien
QFGTPRGTVWPHLQLAQGKLLKDVFRPGDVFFNTGDLLVCDDQGFLRFHDRTGDTFRWKGENVATTEVAEVFEALDFLQEVNVYGVTVPGHEGRAGMAALVLRPPHALDLMQLYTHVSENLPPYARPRFLRLQESLATTETFKQQKVRMANE

SEQ ID NO: 37 - protein, 144 aa, homo sapien
VGSGLRPDTWERFVRRFGPLQVLETYGLTEGNVATINYTGQRGAVGRASWLYKHIFPFSLIRYDVTTGEPIRDPQGHCMATSPGFLRFHDRTGDTFRWKGENVATTEVAEVFEALDFLQEVNVYGVTVPGHEGRAGMAALVLRP

SEQ ID NO: 38 - protein, 229 aa, homo sapien
MAALLLLPLLLLLPLLLLKLHLWPQLRWLPADLAFAVRALCCKRALRARALAAAAADPEGPEGGCSLAWRLAELAQQRAAHTFLIHGSRRFSYSEAERESNRAARAFLRALGWDWGPDGGDSGEGSAGEGERAAPGAGDAAAGSGAEFAGGDGAARGGGAAAPLSPGATVALLLPAGPEFLWLWFGLAKAGLRTAFVPTALRRGPLLHCLRSCGARALVLAPGKAGAPN

SEQ ID NO: 39 - DNA, 20 bp, artificial sequence
  Synthetic sequence; gRNA Recognition Sequence
atcaactacacaggacagcg

SEQ ID NO: 40 - DNA, 20 bp, artificial sequence
  Synthetic sequence; gRNA Recognition Sequence
gagacatatggactgacaga

SEQ ID NO: 41 - DNA, 20 bp, artificial sequence
  Synthetic sequence; gRNA Recognition Sequence
gacggtgttccagtacattg

SEQ ID NO: 42 - DNA, 20 bp, artificial sequence
  Synthetic sequence; gRNA Recognition Sequence
ccagcagggaaccggacatg

SEQ ID NO: 43 - DNA, 20 bp, artificial sequence
  Synthetic sequence; gRNA Recognition Sequence
gcaaatcgctaattccagca

SEQ ID NO: 44 - DNA, 20 bp, artificial sequence
  Synthetic sequence; gRNA Recognition Sequence
agtcccagcctagcgcacgt

SEQ ID NO: 45 - DNA, 20 bp, artificial sequence
  Synthetic sequence; gRNA Recognition Sequence
cctttctcattcacggctcg

SEQ ID NO: 46 - DNA, 20 bp, artificial sequence
  Synthetic sequence; gRNA Recognition Sequence
cgtgtctgttatgctctggg

SEQ ID NO: 47 - DNA, 20 bp, artificial sequence
  Synthetic sequence; gRNA Recognition Sequence
ccatcaactacacaggacag

SEQ ID NO: 48 - DNA, 20 bp, artificial sequence
  Synthetic sequence; gRNA Recognition Sequence
tggactgacagagggcaacg

SEQ ID NO: 49 - DNA, 20 bp, artificial sequence
  Synthetic sequence; gRNA Recognition Sequence
ggagacatatggactgacag

SEQ ID NO: 50 - DNA, 20 bp, artificial sequence
  Synthetic sequence; gRNA Recognition Sequence
aatcgctaattccagcaggg

SEQ ID NO: 51 - DNA, 20 bp, artificial sequence
  Synthetic sequence; gRNA Recognition Sequence
ctgtcctgtgtagttgatgg

SEQ ID NO: 52 - DNA, 20 bp, artificial sequence
  Synthetic sequence; gRNA Recognition Sequence
gcggcgcggagtttgccgga

SEQ ID NO: 53 - DNA, 20 bp, artificial sequence
  Synthetic sequence; gRNA Recognition Sequence
ccgacccggaaggtcccgag

SEQ ID NO: 54 - DNA, 20 bp, artificial sequence
  Synthetic sequence; gRNA Recognition Sequence
cagggcttctatcagctgtg

SEQ ID NO: 55 - DNA, 20 bp, artificial sequence
  Synthetic sequence; gRNA Recognition Sequence
gtgaatgagaaaggtgtgcg

SEQ ID NO: 56 - DNA, 20 bp, artificial sequence
  Synthetic sequence; gRNA Recognition Sequence
ggacatgtggtagagtggga

SEQ ID NO: 57 - DNA, 20 bp, artificial sequence
  Synthetic sequence; gRNA Recognition Sequence
tgggcacaaaggcagtgcgc

SEQ ID NO: 58 - DNA, 20 bp, artificial sequence
  Synthetic sequence; gRNA Recognition Sequence
gactgatccgagcagccttg

(19) United States
(12) Patent Application Publication
(10) Pub. No.: US 2023/0023182 A1
(43) Pub. Date: Jan. 26, 2023

(54) Methods Of Treating Asthma With Solute Carrier Family 27 Member 3 (SLC27A3) Inhibitors

(71) Applicant: Regeneron Pharmaceuticals, Inc., Tarrytown, NY (US)

(72) Inventors: Manuel Allen Revez Ferreira, Tarrytown, NY (US); Joshua Backman, Tarrytown, NY (US); Alexander Li, Tarrytown, NY (US); Goncalo Abecasis, Tarrytown, NY (US); Julie Horowitz, Tarrytown, NY (US); Katherine Siminovitch, Tarrytown, NY (US); Aris Baras, Tarrytown, NY (US)

(21) Appl. No.: 17/854,283

(22) Filed: Jun. 30, 2022

Related U.S. Application Data
(60) Provisional application No. 63/217,913, filed on Jul. 2, 2021.

(51) Int. Cl. C12Q 1/6883; A61K 31/7088

(52) U.S. Cl. CPC C12Q 1/6883; A61K 31/7088; C12Q 2600/156; C12Q 2600/158

(57) ABSTRACT

The present disclosure provides methods of treating a subject having asthma or at risk of developing asthma, and methods of identifying subjects having an increased risk of developing asthma.

### REFERENCE TO SEQUENCE LISTING

This application includes a Sequence Listing filed electronically as a text file named 18923807701SEQ, created on Jun. 25, 2022, with a size of 137 kilobytes. The Sequence Listing is incorporated herein by reference.

### FIELD

The present disclosure relates generally to the treatment of subjects having asthma or at risk of developing asthma with a Solute Carrier Family 27 Member 3 (SLC27A3) inhibitor, and methods of identifying subjects having an increased risk of developing asthma.

### BACKGROUND

Asthma is a chronic lung condition that affects millions of people worldwide. Nearly 25 million people in the United States had asthma in 2018, including approximately 5.5 million children. Globally, an estimated 339 million people have asthma. Airway inflammation, including bronchial hyperresponsiveness, and airway remodeling are predominant features of asthma, a phenotypically heterogeneous chronic respiratory disease. Significant evidence points to a role for aberrant bronchial epithelial cell and immune cell activity in classic asthma.

Current approaches to treat asthma and other inflammatory pulmonary diseases are categorized into two general classes, long-term control medications to achieve and maintain control of persistent disease, and quick-relief medications for treating acute symptoms and exacerbations, most requiring passive drug uptake by target cells through oral or aerosolized delivery. While effective in many patients, a growing number of patients are refractory to current approaches, requiring more improved treatment strategies for controlling disease. A recent approach of bronchial thermoplasty, applying radiofrequency energy to the airway in severe asthma patients, is also used clinically but elicits inflammation at the targeted sites and is associated with spasm and severe asthma exacerbations.

Solute Carrier Family 27 Member 3 (SLC27A3) is a member of a gene family of integral membrane proteins and encodes a protein that is involved in lipid metabolism. This protein has acyl-CoA ligase activity for long-chain and very-long-chain fatty acid. The increased expression of this gene in human neural stem cells derived from induced pluripotent stem cells suggests that it plays an important role in early brain development. Naturally occurring mutations in this gene are associated with autism spectrum disorders.

### SUMMARY

The present disclosure provides methods of treating a subject having asthma or at risk of developing asthma, the methods comprising administering an SLC27A3 inhibitor to the subject.

The present disclosure also provides methods of treating a subject having allergic asthma or at risk of developing allergic asthma, the methods comprising administering an SLC27A3 inhibitor to the subject.

The present disclosure also provides methods of treating a subject having nonallergic asthma or at risk of developing nonallergic asthma, the methods comprising administering an SLC27A3 inhibitor to the subject.

The present disclosure also provides methods of treating a subject having exercise-Induced bronchoconstriction or at risk of developing exercise-induced bronchoconstriction, the methods comprising administering an SLC27A3 inhibitor to the subject.

The present disclosure also provides methods of treating a subject having asthma-chronic obstructive pulmonary disease (COPD) overlap syndrome (ACOS) or at risk of developing ACOS, the methods comprising administering an SLC27A3 inhibitor to the subject.

The present disclosure also provides methods of treating a subject having eosinophilic asthma or at risk of developing eosinophilic asthma, the methods comprising administering an SLC27A3 inhibitor to the subject.

The present disclosure also provides methods of treating a subject having childhood asthma or at risk of developing childhood asthma, the methods comprising administering an SLC27A3 inhibitor to the subject.

The present disclosure also provides methods of treating a subject having occupational asthma or at risk of developing occupational asthma, the methods comprising administering an SLC27A3 inhibitor to the subject.

The present disclosure also provides methods of treating a subject with a therapeutic agent that treats or prevents asthma, wherein the subject has asthma or is at risk of developing asthma, the methods comprising the steps of: determining whether the subject has an SLC27A3 variant nucleic acid molecule encoding an SLC27A3 predicted loss-of-function polypeptide by: obtaining or having obtained a biological sample from the subject; and performing or having performed a sequence analysis on the biological sample to determine if the subject has a genotype comprising the SLC27A3 variant nucleic acid molecule encoding the SLC27A3 predicted loss-of-function polypeptide; and: i) administering or continuing to administer the therapeutic agent that treats or prevents asthma in a standard dosage amount to a subject that is SLC27A3 reference, and/or administering an SLC27A3 inhibitor to the subject; ii) administering or continuing to administer the therapeutic agent that treats or prevents asthma in an amount that is the same as or less than a standard dosage amount to a subject that is heterozygous for the SLC27A3 variant nucleic acid molecule, and/or administering an SLC27A3 inhibitor to the subject; or iii) administering or continuing to administer the therapeutic agent that treats or prevents asthma in an amount that is the same as or less than a standard dosage amount to a subject that is homozygous for the SLC27A3 variant nucleic acid molecule; wherein the presence of a genotype having the SLC27A3 variant nucleic acid molecule encoding the SLC27A3 predicted loss-of-function polypeptide indicates the subject has a decreased risk of developing asthma.

The present disclosure also provides methods of identifying a subject having an increased risk of developing asthma, the methods comprising: determining or having determined the presence or absence of an SLC27A3 variant nucleic acid molecule encoding an SLC27A3 predicted loss-of-function polypeptide in a biological sample obtained from the subject; when the subject is SLC27A3 reference, then the subject has an increased risk of developing asthma; and when the subject is heterozygous or homozygous for the SLC27A3 variant nucleic acid molecule encoding the SLC27A3 predicted loss-of-function polypeptide, then the subject has a decreased risk of developing asthma.

The present disclosure also provides therapeutic agents that treat or prevent asthma for use in the treatment or prevention of asthma in a subject having: an SLC27A3 variant genomic nucleic acid molecule encoding an SLC27A3 predicted loss-of-function polypeptide; an SLC27A3 variant mRNA molecule encoding an SLC27A3 predicted loss-of-function polypeptide; or an SLC27A3 variant cDNA molecule encoding an SLC27A3 predicted loss-of-function polypeptide.

The present disclosure also provides SLC27A3 inhibitors for use in the treatment or prevention of asthma in a subject that: a) is reference for an SLC27A3 genomic nucleic acid molecule, an SLC27A3 mRNA molecule, or an SLC27A3 cDNA molecule; or b) is heterozygous for: i) an SLC27A3 variant genomic nucleic acid molecule encoding an SLC27A3 predicted loss-of-function polypeptide; ii) an SLC27A3 variant mRNA molecule encoding an SLC27A3 predicted loss-of-function polypeptide; or iii) an SLC27A3 variant cDNA molecule encoding an SLC27A3 predicted loss-of-function polypeptide.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that SLC27A3 variant nucleic acid molecules encoding SLC27A3 predicted loss-of-function polypeptides (whether these variations are homozygous or heterozygous in a particular subject) associate with a decreased risk of developing asthma. It is believed that SLC27A3 variant nucleic acid molecules encoding SLC27A3 predicted loss-of-function polypeptides have not been associated with asthma. Moreover, the identification by the present disclosure of the association between additional variants and gene burden masks indicates that SLC27A3 itself (rather than linkage disequilibrium with variants in another gene) is responsible for a protective effect in asthma.

Therefore, subjects that are SLC27A3 reference or heterozygous for SLC27A3 variant nucleic acid molecules encoding SLC27A3 predicted loss-of-function polypeptides may be treated with an SLC27A3 inhibitor such that asthma is inhibited or prevented, the symptoms thereof are reduced or prevented, and/or development of symptoms is repressed or prevented. It is also believed that such subjects having asthma may further be treated with therapeutic agents that treat or prevents asthma.

For purposes of the present disclosure, any particular subject, such as a human, can be categorized as having one of three SLC27A3 genotypes: i) SLC27A3 reference; ii) heterozygous for an SLC27A3 variant nucleic acid molecule encoding an SLC27A3 predicted loss-of-function polypeptide; or iii) homozygous for an SLC27A3 variant nucleic acid molecules encoding an SLC27A3 predicted loss-of-function polypeptide. A subject is SLC27A3 reference when the subject does not have a copy of an SLC27A3 variant nucleic acid molecules encoding an SLC27A3 predicted loss-of-function polypeptide. A subject is heterozygous for an SLC27A3 variant nucleic acid molecules encoding an SLC27A3 predicted loss-of-function polypeptide when the subject has a single copy of an SLC27A3 variant nucleic acid molecules encoding an SLC27A3 predicted loss-of-function polypeptide. An SLC27A3 variant nucleic acid molecule encoding an SLC27A3 predicted loss-of-function polypeptide is any nucleic acid molecule (such as, a genomic nucleic acid molecule, an mRNA molecule, or a cDNA molecule) encoding a variant SLC27A3 polypeptide having a partial loss-of-function, a complete loss-of-function, a predicted partial loss-of-function, or a predicted complete loss-of-function. A subject who has an SLC27A3 polypeptide having a partial loss-of-function (or predicted partial loss-of-function) is hypomorphic for SLC27A3. A subject is homozygous for an SLC27A3 variant nucleic acid molecule encoding an SLC27A3 predicted loss-of-function polypeptide when the subject has two copies (same or different) of an SLC27A3 variant nucleic acid molecule encoding an SLC27A3 predicted loss-of-function polypeptide.

For subjects that are genotyped or determined to be SLC27A3 reference, such subjects have an increased risk of developing asthma, such as allergic asthma, nonallergic asthma, exercise-induced bronchoconstriction, ACOS, eosinophilic asthma, childhood asthma, and/or occupational asthma. For subjects that are genotyped or determined to be either SLC27A3 reference or heterozygous for an SLC27A3 variant nucleic acid molecule encoding an SLC27A3 predicted loss-of-function polypeptide, such subjects can be treated with an SLC27A3 inhibitor.

In any of the embodiments described herein, the subject in whom asthma is prevented by administering the SLC27A3 inhibitor can be anyone at risk for developing asthma including, but not limited to, subjects with allergies, respiratory infections, genetic predisposition to asthma or obese subjects. In some embodiments, any subject can be at risk of developing asthma. In some embodiments, administering an SLC27A3 inhibitor may be carried out to prevent development of an additional asthma in a subject who has already had asthma.

In any of the embodiments described herein, the SLC27A3 variant nucleic acid molecules encoding SLC27A3 predicted loss-of-function polypeptides can be any nucleic acid molecule (such as, for example, genomic nucleic acid molecule, mRNA molecule, or cDNA molecule) encoding an SLC27A3 variant polypeptide having a partial loss-of-function, a complete loss-of-function, a predicted partial loss-of-function, or a predicted complete loss-of-function. In some embodiments, the SLC27A3 variant nucleic acid molecule encoding an SLC27A3 predicted loss-of-function polypeptide is associated with a reduced in vitro response to SLC27A3 ligands compared with reference SLC27A3. In some embodiments, the SLC27A3 variant nucleic acid molecule encoding an SLC27A3 predicted loss-of-function polypeptide is an SLC27A3 variant that results or is predicted to result in a premature truncation of an SLC27A3 polypeptide compared to the human reference genome sequence. In some embodiments, the SLC27A3 variant nucleic acid molecule encoding an SLC27A3 predicted loss-of-function polypeptide is a variant that is predicted to be damaging by in vitro prediction algorithms such as Polyphen, SIFT, or similar algorithms. In some embodiments, the SLC27A3 variant nucleic acid molecule encoding an SLC27A3 predicted loss-of-function polypeptide is a variant that causes or is predicted to cause a nonsynonymous amino-acid substitution in SLC27A3 and whose allele frequency is less than 1/100 alleles in the population from which the subject is selected. In some embodiments, the SLC27A3 variant nucleic acid molecule encoding an SLC27A3 predicted loss-of-function polypeptide is any rare variant (allele frequency <0.1%; or 1 in 1,000 alleles), or any splice-site, stop-gain, start-loss, stop-loss, frameshift, or in-frame indel, or other frameshift SLC27A3 variant.

In any of the embodiments described herein, the SLC27A3 predicted loss-of-function polypeptide can be any SLC27A3 polypeptide having a partial loss-of-function, a complete loss-of-function, a predicted partial loss-of-function, or a predicted complete loss-of-function.

In any of the embodiments described herein, the SLC27A3 variant nucleic acid molecule encoding an SLC27A3 predicted loss-of-function polypeptide can include variations at positions of chromosome 1 using the nucleotide sequence of the SLC27A3 reference genomic nucleic acid molecule (SEQ ID NO:1; ENSG00000143554.14, located at chr1:153,774,354-153,780,157 in the GRCh38/hg38 human genome assembly) as a reference sequence.

Any one or more (i.e., any combination) of the SLC27A3 variant nucleic acid molecules encoding SLC27A3 predicted loss-of-function polypeptides can be used within any of the methods described herein to determine whether a subject has an increased risk of developing asthma. The combinations of particular variants can form a mask used for statistical analysis of the particular correlation of SLC27A3 and decreased risk of developing asthma.

In any of the embodiments described herein, the asthma is allergic asthma, nonallergic asthma, exercise-induced bronchoconstriction, ACOS, eosinophilic asthma, childhood asthma, and/or occupational asthma. In some embodiments, the asthma is allergic asthma. In some embodiments, the asthma is nonallergic asthma. In some embodiments, the asthma is exercise-induced bronchoconstriction. In some embodiments, the asthma is ACOS. In some embodiments, the asthma is eosinophilic asthma. In some embodiments, the asthma is childhood asthma. In some embodiments, the asthma is occupational asthma.

Symptoms of allergic asthma include, but are not limited to, shortness of breath, chest tightness, cough, especially at night, and wheezing.

Symptoms of nonallergic asthma include, but are not limited to, shortness of breath, chest tightness, cough, especially at night, and wheezing.

Symptoms of exercise-induced bronchoconstriction include, but are not limited to, shortness of breath, chest tightness, cough.

Symptoms of ACOS include, but are not limited to, difficulty breathing, excess mucus (more than usual), feeling tired, frequent coughing, frequent shortness of breath, and wheezing.

Symptoms of eosinophilic asthma include, but are not limited to, shortness of breath, chest tightness, cough, especially at night, wheezing, chronic nasal and sinus inflammation, and nasal polyps.

Symptoms of childhood asthma include, but are not limited to, coughing during sleep, repeated instances of bronchitis or pneumonia, coughing or wheezing as the result of laughing, crying, or playing, and loud or fast breathing.

Symptoms of occupational asthma include, but are not limited to, wheezing, shortness of breath, runny nose, nasal congestion, eye irritation, and chest tightness, which may get worse during exposure to the irritant(s) at work.

The present disclosure provides methods of treating a subject having asthma or at risk of developing asthma, the methods comprising administering an SLC27A3 inhibitor to the subject.

The present disclosure also provides methods of treating a subject having allergic asthma or at risk of developing allergic asthma, the methods comprising administering an SLC27A3 inhibitor to the subject.

The present disclosure also provides methods of treating a subject having nonallergic asthma or at risk of developing nonallergic asthma, the methods comprising administering an SLC27A3 inhibitor to the subject.

The present disclosure also provides methods of treating a subject having exercise-induced bronchoconstriction or at risk of developing exercise-induced bronchoconstriction, the methods comprising administering an SLC27A3 inhibitor to the subject.

The present disclosure also provides methods of treating a subject having ACOS or at risk of developing ACOS, the methods comprising administering an SLC27A3 inhibitor to the subject.

The present disclosure also provides methods of treating a subject having eosinophilic asthma or at risk of developing eosinophilic asthma, the methods comprising administering an SLC27A3 inhibitor to the subject.

The present disclosure also provides methods of treating a subject having childhood asthma or at risk of developing childhood asthma, the methods comprising administering an SLC27A3 inhibitor to the subject.

The present disclosure also provides methods of treating a subject having occupational asthma or at risk of developing occupational asthma, the methods comprising administering an SLC27A3 inhibitor to the subject.

In some embodiments, the SLC27A3 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an SLC27A3 nucleic acid molecule. In some embodiments, the antisense RNA, siRNA, or shRNA hybridizes to a sequence within an SLC27A3 genomic nucleic acid molecule or mRNA molecule and decreases expression of the SLC27A3 polypeptide in a cell in the subject. In some embodiments, the SLC27A3 inhibitor comprises an antisense molecule that hybridizes to an SLC27A3 genomic nucleic acid molecule or mRNA molecule and decreases expression of the SLC27A3 polypeptide in a cell in the subject. In some embodiments, the SLC27A3 inhibitor comprises an siRNA that hybridizes to an SLC27A3 genomic nucleic acid molecule or mRNA molecule and decreases expression of the SLC27A3 polypeptide in a cell in the subject. In some embodiments, the SLC27A3 inhibitor comprises an shRNA that hybridizes to an SLC27A3 genomic nucleic acid molecule or mRNA molecule and decreases expression of the SLC27A3 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:

Sense: mN*mN*/i2FN/mN/i2FN/mN/i2FN/mN/i2FN/mN/i2FN/mN/i2FN/mN/i2FN/mN/i2FN/*mN*/32FN/

### Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the SLC27A3 inhibitor comprises a nuclease agent that induces one or more nicks or double-strand breaks at a recognition sequence(s) or a DNA-binding protein that binds to a recognition sequence within an SLC27A3 genomic nucleic acid molecule. The recognition sequence can be located within a coding region of the SLC27A3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SLC27A3 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an SLC27A3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of SLC27A3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an SLC27A3 genomic nucleic acid molecule or it can be a nickase that creates a single-strand break in an SLC27A3 genomic nucleic acid molecule. Additional examples of Cas proteins include, but are not limited to, Cas1, Cas1B, Cast, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (Cas6), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of SLC27A3 genomic nucleic acid molecules can be generated by contacting a cell with a Cas protein and one or more gRNAs that hybridize to one or more gRNA recognition sequences within a target genomic locus in the SLC27A3 genomic nucleic acid molecule. For example, a gRNA recognition sequence can be located within a region of SEQ ID NO:1. The gRNA recognition sequence can include or be proximate to the start codon of an SLC27A3 genomic nucleic acid molecule or the stop codon of an SLC27A3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an SLC27A3 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an SLC27A3 genomic nucleic acid molecule. An exemplary gRNA is a gRNA effective to direct a Cas enzyme to bind to or cleave an SLC27A3 genomic nucleic acid molecule, wherein the gRNA comprises a DNA-targeting segment that hybridizes to a gRNA recognition sequence within the SLC27A3 genomic nucleic acid molecule. Exemplary gRNAs comprise a DNA-targeting segment that hybridizes to a gRNA recognition sequence present within an SLC27A3 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the human SLC27A3 reference gene are set forth in Table 1 as SEQ ID NOs:39-58.

TABLE 1
Guide RNA Recognition Sequences
Near the SLC27A3 Gene

| | | SEQ |
|---|---|---|
| | gRNA Recognition | ID |
| Strand | Sequence | NO: |
| + | ATCAACTACACAGGACAGCG | 39 |
| + | GAGACATATGGACTGACAGA | 40 |
| + | GACGGTGTTCCAGTACATTG | 41 |
| − | CCAGCAGGGAACCGGACATG | 42 |
| − | GCAAATCGCTAATTCCAGCA | 43 |
| − | AGTCCCAGCCTAGCGCACGT | 44 |
| + | CCTTTCTCATTCACGGCTCG | 45 |
| − | CGTGTCTGTTATGCTCTGGG | 46 |
| + | CCATCAACTACACAGGACAG | 47 |
| + | TGGACTGACAGAGGGCAACG | 48 |
| + | GGAGACATATGGACTGACAG | 49 |
| − | AATCGCTAATTCCAGCAGGG | 50 |
| − | CTGTCCTGTGTAGTTGATGG | 51 |
| + | GCGGCGCGGAGTTTGCCGGA | 52 |
| + | CCGACCCGGAAGGTCCCGAG | 53 |
| + | CAGGGCTTCTATCAGCTGTG | 54 |
| − | GTGAATGAGAAAGGTGTGCG | 55 |
| − | GGACATGTGGTAGAGTGGGA | 56 |
| − | TGGGCACAAAGGCAGTGCGC | 57 |
| − | GACTGATCCGAGCAGCCTTG | 58 |

The Cas protein and the gRNA form a complex, and the Cas protein cleaves the target SLC27A3 genomic nucleic acid molecule. The Cas protein can cleave the nucleic acid molecule at a site within or outside of the nucleic acid sequence present in the target SLC27A3 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the SLC27A3 genomic nucleic acid molecule to which a DNA-targeting segment of a gRNA will bind.

Such methods can result, for example, in an SLC27A3 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SLC27A3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods of treatment and/or prevention further comprise detecting the presence or absence of an SLC27A3 variant nucleic acid molecule encoding an SLC27A3 predicted loss-of-function polypeptide in a biological sample from the subject. As used throughout the present disclosure, a “SLC27A3 variant nucleic acid molecule encoding an SLC27A3 predicted loss-of-function polypeptide” is any SLC27A3 nucleic acid molecule (such as, for example, genomic nucleic acid molecule, mRNA molecule, or cDNA molecule) encoding an SLC27A3 polypeptide having a partial loss-of-function, a complete loss-of-function, a predicted partial loss-of-function, or a predicted complete loss-of-function.

The present disclosure also provides methods of treating a subject with a therapeutic agent that treats or prevents asthma, wherein the subject has asthma or is at risk of developing asthma. In some embodiments, the methods comprise determining whether the subject has an SLC27A3 variant nucleic acid molecule encoding an SLC27A3 predicted loss-of-function polypeptide by obtaining or having obtained a biological sample from the subject, and performing or having performed a sequence analysis on the biological sample to determine if the subject has a genotype comprising the SLC27A3 variant nucleic acid molecule encoding an SLC27A3 predicted loss-of-function polypeptide. In some embodiments, the methods further comprise administering or continuing to administer the therapeutic agent that treats or prevents asthma in a standard dosage amount to a subject that is SLC27A3 reference, and/or administering an SLC27A3 inhibitor to the subject. In some embodiments, the methods further comprise administering or continuing to administer the therapeutic agent that treats or prevents asthma in an amount that is the same as or less than a standard dosage amount to a subject that is heterozygous for the SLC27A3 variant nucleic acid molecule, and/or administering an SLC27A3 inhibitor to the subject. In some embodiments, the methods further comprise administering or continuing to administer the therapeutic agent that treats or prevents asthma in an amount that is the same as or less than a standard dosage amount to a subject that is homozygous for the SLC27A3 variant nucleic acid molecule. The presence of a genotype having the SLC27A3 variant nucleic acid molecule encoding the SLC27A3 predicted loss-of-function polypeptide indicates the subject has a decreased risk of developing asthma. In some embodiments, the subject is SLC27A3 reference. In some embodiments, the subject is heterozygous for an SLC27A3 variant nucleic acid molecule encoding an SLC27A3 predicted loss-of-function polypeptide.

For subjects that are genotyped or determined to be either SLC27A3 reference or heterozygous for an SLC27A3 variant nucleic acid molecule encoding an SLC27A3 predicted loss-of-function polypeptide, such subjects can be administered an SLC27A3 inhibitor, as described herein.

Detecting the presence or absence of an SLC27A3 variant nucleic acid molecule encoding an SLC27A3 predicted loss-of-function polypeptide in a biological sample from a subject and/or determining whether a subject has an SLC27A3 variant nucleic acid molecule encoding an SLC27A3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when the subject is SLC27A3 reference, the subject is administered a therapeutic agent that treats or prevents asthma in a standard dosage amount. In some embodiments, when the subject is heterozygous for an SLC27A3 variant nucleic acid molecule encoding an SLC27A3 predicted loss-of-function polypeptide, the subject is administered a therapeutic agent that treats or prevents asthma in a dosage amount that is the same as or less than a standard dosage amount.

In some embodiments, the treatment and/or prevention methods further comprise detecting the presence or absence of an SLC27A3 predicted loss-of-function polypeptide in a biological sample from the subject. In some embodiments, when the subject does not have an SLC27A3 predicted loss-of-function polypeptide, the subject is also administered a therapeutic agent that treats or prevents asthma in a standard dosage amount. In some embodiments, when the subject has an SLC27A3 predicted loss-of-function polypeptide, the subject is also administered a therapeutic agent that treats or prevents asthma in a dosage amount that is the same as or less than a standard dosage amount.

The present disclosure also provides methods of treating a subject with a therapeutic agent that treats or prevents asthma, wherein the subject has asthma or is at risk of developing asthma. In some embodiments, the method comprises determining whether the subject has an SLC27A3 predicted loss-of-function polypeptide by obtaining or having obtained a biological sample from the subject, and performing or having performed an assay on the biological sample to determine if the subject has an SLC27A3 predicted loss-of-function polypeptide. When the subject does not have an SLC27A3 predicted loss-of-function polypeptide, the therapeutic agent that treats or prevents asthma is administered or continued to be administered to the subject in a standard dosage amount, and/or an SLC27A3 inhibitor is administered to the subject. When the subject has an SLC27A3 predicted loss-of-function polypeptide, the therapeutic agent that treats or prevents asthma is administered or continued to be administered to the subject in an amount that is the same as or less than a standard dosage amount, and/or an SLC27A3 inhibitor is administered to the subject. The presence of an SLC27A3 predicted loss-of-function polypeptide indicates the subject has a decreased risk of developing asthma. In some embodiments, the subject has an SLC27A3 predicted loss-of-function polypeptide. In some embodiments, the subject does not have an SLC27A3 predicted loss-of-function polypeptide.

Detecting the presence or absence of an SLC27A3 predicted loss-of-function polypeptide in a biological sample from a subject and/or determining whether a subject has an SLC27A3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

In some embodiments, the SLC27A3 inhibitor is a small molecule. In some embodiments, the SLC27A3 inhibitor is an anti-SLC27A3 antibody.

Examples of therapeutic agents that treat or prevent asthma include, but are not limited to, inhaled steroids (such as, mometasone, ciclesonide, fluticasone, budesonide, flunisolide, beclomethasone, and triamcinolone); combination medications (such as, fluticasone and salmeterol, mometasone and formoterol, budesonide and formoterol, and fluticasone furoate and vilanterol combination); anticholinergic maintenance medications (such as, aclidinium, glycopyrronium, ipratropium, tiotropium, and umeclidinium); leukotriene modifiers (such as, zafirlukast, montelukast), and zileuton); and biologic immunomodulators (such as, mepolizumab, reslizumab, benralizumab, omalizumab, and dupilumab).

In some embodiments, the therapeutic agent that treats or prevents asthma is an inhaled steroid. In some embodiments, the therapeutic agent that treats or prevents asthma is a combination medication. In some embodiments, the therapeutic agent that treats or prevents asthma is an anticholinergic maintenance medication. In some embodiments, the therapeutic agent that treats or prevents asthma is a leukotriene modifier. In some embodiments, the therapeutic agent that treats or prevents asthma is a biologic immunomodulator.

In some embodiments, the inhaled steroid is mometasone. In some embodiments, the inhaled steroid is ciclesonide. In some embodiments, the inhaled steroid is fluticasone. In some embodiments, the inhaled steroid is budesonide. In some embodiments, the inhaled steroid is flunisolide. In some embodiments, the inhaled steroid is beclomethasone. In some embodiments, the inhaled steroid is triamcinolone. In some embodiments, the combination medication is fluticasone and salmeterol. In some embodiments, the combination medication is mometasone and formoterol. In some embodiments, the combination medication is budesonide and formoterol. In some embodiments, the combination medication is fluticasone furoate and vilanterol. In some embodiments, the anticholinergic maintenance medication is aclidinium. In some embodiments, the anticholinergic maintenance medication is glycopyrronium. In some embodiments, the anticholinergic maintenance medication is ipratropium. In some embodiments, the anticholinergic maintenance medication is tiotropium. In some embodiments, the anticholinergic maintenance medication is umeclidinium. In some embodiments, the leukotriene modifier is zafirlukast. In some embodiments, the leukotriene modifier is montelukast. In some embodiments, the leukotriene modifier is zileuton. In some embodiments, the biologic immunomodulator is mepolizumab. In some embodiments, the biologic immunomodulator is reslizumab. In some embodiments, the biologic immunomodulator is benralizumab. In some embodiments, the biologic immunomodulator is omalizumab. In some embodiments, the biologic immunomodulator is dupilumab).

In some embodiments, the dose of the therapeutic agents that treat or prevent asthma can be decreased by about 10%, by about 20%, by about 30%, by about 40%, by about 50%, by about 60%, by about 70%, by about 80%, or by about 90% for subjects that are heterozygous for an SLC27A3 variant nucleic acid molecule encoding an SLC27A3 predicted loss-of-function polypeptide (i.e., a less than the standard dosage amount) compared to subjects that are SLC27A3 reference (who may receive a standard dosage amount). In some embodiments, the dose of the therapeutic agents that treat or prevent asthma can be decreased by about 10%, by about 20%, by about 30%, by about 40%, or by about 50%. In addition, the subjects that are heterozygous for an SLC27A3 variant nucleic acid molecule encoding an SLC27A3 predicted loss-of-function polypeptide can be administered less frequently compared to subjects that are SLC27A3 reference.

In some embodiments, the dose of the therapeutic agents that treat or prevent asthma can be decreased by about 10%, by about 20%, by about 30%, by about 40%, by about 50%, for subjects that are homozygous for an SLC27A3 variant nucleic acid molecule encoding an SLC27A3 predicted loss-of-function polypeptide compared to subjects that are heterozygous for an SLC27A3 variant nucleic acid molecule encoding an SLC27A3 predicted loss-of-function polypeptide. In some embodiments, the dose of the therapeutic agents that treat or prevent asthma can be decreased by about 10%, by about 20%, by about 30%, by about 40%, or by about 50%. In addition, the dose of therapeutic agents that treat or prevent asthma in subjects that are homozygous for an SLC27A3 variant nucleic acid molecule encoding an SLC27A3 predicted loss-of-function polypeptide can be administered less frequently compared to subjects that are heterozygous for an SLC27A3 variant nucleic acid molecule encoding an SLC27A3 predicted loss-of-function polypeptide.

Administration of the therapeutic agents that treat or prevent asthma and/or SLC27A3 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the therapeutic agents that treat or prevent asthma and/or SLC27A3 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in asthma, a decrease/reduction in the severity of asthma (such as, for example, a reduction or inhibition of development of asthma), a decrease/reduction in symptoms and asthma-related effects, delaying the onset of symptoms and asthma-related effects, reducing the severity of symptoms of asthma-related effects, reducing the number of symptoms and asthma-related effects, reducing the latency of symptoms and asthma-related effects, an amelioration of symptoms and asthma-related effects, reducing secondary symptoms, reducing secondary infections, preventing relapse to asthma, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of asthma development/progression (such as, for example, a complete or partial avoidance/inhibition or a delay), and an increased survival time of the affected host animal, following administration of a therapeutic protocol. Treatment of asthma encompasses the treatment of a subject already diagnosed as having any form of asthma at any clinical stage or manifestation, the delay of the onset or evolution or aggravation or deterioration of the symptoms or signs of asthma, and/or preventing and/or reducing the severity of asthma.

The present disclosure also provides methods of identifying a subject having an increased risk of developing asthma. In some embodiments, the method comprises determining or having determined in a biological sample obtained from the subject the presence or absence of an SLC27A3 variant nucleic acid molecule (such as a genomic nucleic acid molecule, mRNA molecule, and/or cDNA molecule) encoding an SLC27A3 predicted loss-of-function polypeptide encoding an SLC27A3 polypeptide. When the subject lacks an SLC27A3 variant nucleic acid molecule encoding an SLC27A3 predicted loss-of-function polypeptide (i.e., the subject is genotypically categorized as an SLC27A3 reference), then the subject has an increased risk of developing asthma. When the subject has an SLC27A3 variant nucleic acid molecule encoding an SLC27A3 predicted loss-of-function polypeptide (i.e., the subject is heterozygous or homozygous for an SLC27A3 variant nucleic acid molecule encoding an SLC27A3 predicted loss-of-function polypeptide), then the subject has a decreased risk of developing asthma.

Having a single copy of an SLC27A3 variant nucleic acid molecule encoding an SLC27A3 predicted loss-of-function polypeptide is more protective of a subject from developing asthma than having no copies of an SLC27A3 variant nucleic acid molecule encoding an SLC27A3 predicted loss-of-function polypeptide. Without intending to be limited to any particular theory or mechanism of action, it is believed that a single copy of an SLC27A3 variant nucleic acid molecule encoding an SLC27A3 predicted loss-of-function polypeptide (i.e., heterozygous for an SLC27A3 variant nucleic acid molecule encoding an SLC27A3 predicted loss-of-function polypeptide) is protective of a subject from developing asthma, and it is also believed that having two copies of an SLC27A3 variant nucleic acid molecule encoding an SLC27A3 predicted loss-of-function polypeptide (i.e., homozygous for an SLC27A3 variant nucleic acid molecule encoding an SLC27A3 predicted loss-of-function polypeptide) may be more protective of a subject from developing asthma, relative to a subject with a single copy. Thus, in some embodiments, a single copy of an SLC27A3 variant nucleic acid molecule encoding an SLC27A3 predicted loss-of-function polypeptide may not be completely protective, but instead, may be partially or incompletely protective of a subject from developing asthma. While not desiring to be bound by any particular theory, there may be additional factors or molecules involved in the development of asthma that are still present in a subject having a single copy of an SLC27A3 variant nucleic acid molecule encoding an SLC27A3 predicted loss-of-function polypeptide, thus resulting in less than complete protection from the development of asthma.

Determining whether a subject has an SLC27A3 variant nucleic acid molecule encoding an SLC27A3 predicted loss-of-function polypeptide in a biological sample from a subject and/or determining whether a subject has an SLC27A3 variant nucleic acid molecule encoding an SLC27A3 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing asthma, the subject is administered a therapeutic agent that treats or prevents asthma, and/or an SLC27A3 inhibitor, as described herein. For example, when the subject is SLC27A3 reference, and therefore has an increased risk of developing asthma, the subject is administered an SLC27A3 inhibitor. In some embodiments, such a subject is also administered a therapeutic agent that treats or prevents asthma. In some embodiments, when the subject is heterozygous for an SLC27A3 variant nucleic acid molecule encoding an SLC27A3 predicted loss-of-function polypeptide, the subject is administered the therapeutic agent that treats or prevents asthma in a dosage amount that is the same as or less than a standard dosage amount, and is also administered an SLC27A3 inhibitor. In some embodiments, such a subject is also administered a therapeutic agent that treats or prevents asthma. In some embodiments, when the subject is homozygous for an SLC27A3 variant nucleic acid molecule encoding an SLC27A3 predicted loss-of-function polypeptide, the subject is administered the therapeutic agent that treats or prevents asthma in a dosage amount that is the same as or less than a standard dosage amount. In some embodiments, the subject is SLC27A3 reference. In some embodiments, the subject is heterozygous for an SLC27A3 variant nucleic acid molecule encoding an SLC27A3 predicted loss-of-function polypeptide. In some embodiments, the subject is homozygous for an SLC27A3 variant nucleic acid molecule encoding an SLC27A3 predicted loss-of-function polypeptide.

In some embodiments, any of the methods described herein can further comprise determining the subject's aggregate burden of having an SLC27A3 variant nucleic acid molecule encoding an SLC27A3 predicted loss-of-function polypeptide, and/or an SLC27A3 predicted loss-of-function variant polypeptide associated with a decreased risk of developing asthma. The aggregate burden is the sum of all variants in the SLC27A3 gene, which can be carried out in an association analysis with asthma. In some embodiments, the subject is homozygous for one or more SLC27A3 variant nucleic acid molecules encoding an SLC27A3 predicted loss-of-function polypeptide associated with a decreased risk of developing asthma. In some embodiments, the subject is heterozygous for one or more SLC27A3 variant nucleic acid molecules encoding an SLC27A3 predicted loss-of-function polypeptide associated with a decreased risk of developing asthma. The result of the association analysis suggests that SLC27A3 variant nucleic acid molecules encoding an SLC27A3 predicted loss-of-function polypeptide are associated with decreased risk of developing asthma. When the subject has a lower aggregate burden, the subject is at a higher risk of developing asthma and the subject is administered or continued to be administered the therapeutic agent that treats or prevents asthma in a standard dosage amount, and/or an SLC27A3 inhibitor. When the subject has a greater aggregate burden, the subject is at a lower risk of developing asthma and the subject is administered or continued to be administered the therapeutic agent that treats or prevents asthma in an amount that is the same as or less than the standard dosage amount. The greater the aggregate burden, the lower the risk of developing asthma.

SLC27A3 variants that can be used in the aggregate burden analysis include any one or more, or any combination, of the following:

| Variant | rsID | Transcript IDs |
|---|---|---|
| 1:153775115:T:A | | ENST00000271857 |
| 1:153775116:G:T | | ENST00000271857 |
| 1:153775116:G:C | | ENST00000271857 |
| 1:153775116:G:A | | ENST00000271857 |
| 1:153775117:C:T | | ENST00000271857 |
| 1:153775120:A:T | | ENST00000271857 |
| 1:153775122:A:C | | ENST00000271857 |
| 1:153775123:C:T | | ENST00000271857 |
| 1:153775124:C:T | | ENST00000271857 |
| 1:153775125:GC:G | | ENST00000271857 |
| 1:153775126:C:T | rs1444740227 | ENST00000271857 |
| 1:153775127:C:T | | ENST00000271857 |
| 1:153775129:A:T | | ENST00000271857 |
| 1:153775132:C:T | rs1435961456 | ENST00000271857 |
| 1:153775133:C:T | rs549168715 | ENST00000271857 |
| 1:153775135:A:G | rs200878053 | ENST00000271857 |
| 1:153775143:C:G | rs531151181 | ENST00000271857 |
| 1:153775145:T:TA | | ENST00000271857 |
| 1:153775145TAG: | | ENST00000271857 |
| T | | |
| 1:153775147:G:T | | ENST00000271857 |
| 1:153775147:G:GA | rs774298665 | ENST00000271857 |
| 1:153775148:A:G | | ENST00000271857 |
| 1:153775148:A:T | | ENST00000271857 |
| 1:153775148:A:C | rs1182659153 | ENST00000271857 |
| 1:153775149:A:ATT | | ENST00000271857 |
| CT | | |
| 1:153775149:A:C | | ENST00000271857 |
| 1:153775150:G:A | rs891823496 | ENST00000271857 |
| 1:153775152:C:CA | | ENST00000271857 |
| 1:153775153:A:G | | ENST00000271857 |
| 1:153775155:A:C | rs1320905930 | ENST00000271857 |
| 1:153775156:CAT:C | rs1043724437 | ENST00000271857 |
| 1:153775156:C:CAT | rs766163606 | ENST00000271857 |
| 1:153775156:C:T | rs370853869 | ENST00000271857 |
| 1:153775157:A:G | rs1447846775 | ENST00000271857 |
| 1:153775159:A:G | | ENST00000271857 |
| 1:153775162:C:G | | ENST00000271857 |
| 1:153775163:C:T | rs750637904 | ENST00000271857 |
| 1:153775165:G:A | rs571550476 | ENST00000271857 |
| 1:153775168:A:G | | ENST00000271857 |
| 1:153775169:T:G | | ENST00000271857 |
| 1:153775172:A:G | | ENST00000271857 |
| 1:153775173:G:C | | ENST00000271857 |
| 1:153775174:G:C | rs1289328759 | ENST00000271857 |
| 1:153775175:G:A | rs1233049780 | ENST00000271857 |
| 1:153775178:G:A | rs539067087 | ENST00000271857 |
| 1:153775180:C:T | rs751624762 | ENST00000271857 |
| 1:153775181:G:A | | ENST00000271857 |
| 1:153775181:G:C | | ENST00000271857 |
| 1:153775183:G:A | rs1441760883 | ENST00000271857 |
| 1:153775184:A:AG | rs1181619421 | ENST00000271857 |
| C | | |
| 1:153775184:A:G | rs1455534085 | ENST00000271857 |
| 1:153775188:C:G | | ENST00000271857 |
| 1:153775189:C:T | rs1244753692 | ENST00000271857 |
| 1:153775189:C:A | | ENST00000271857 |
| 1:153775192:C:A | | ENST00000271857 |
| 1:153775196:G:A | | ENST00000271857 |
| 1:153775198:C:G | rs575288664 | ENST00000271857 |
| 1:153775198:C:T | rs575288664 | ENST00000271857 |
| 1:153775199:C:CT | rs894899211 | ENST00000271857 |
| GGTAGGG | | |
| 1:153775199:C:T | | ENST00000271857 |
| 1:153775199:C:G | rs1451767635 | ENST00000271857 |
| 1:153775201:G:A | | ENST00000271857 |
| 1:153775202:G:C | | ENST00000271857 |
| 1:153775202:G:A | rs1192500304 | ENST00000271857 |
| 1:153775205:G:A | rs1160366367 | ENST00000271857 |
| 1:153775205:G:C | | ENST00000271857 |
| 1:153775207:G:A | | ENST00000271857 |
| 1:153775207:G:T | | ENST00000271857 |
| 1:153775207:G:C | rs1246823627 | ENST00000271857 |
| 1:153775211:G:A | | ENST00000271857 |
| 1:153775212:AGCC | | ENST00000271857 |
| GCGAGCAGTCCGG | | |
| GAGGGAGCGC:A | | |
| 1:153775213:G:A | rs778518509 | ENST00000271857 |
| 1:153775214:C:A | rs542735734 | ENST00000271857 |
| 1:153775214:C:T | rs542735734 | ENST00000271857 |
| 1:153775216:G:A | rs1210099263 | ENST00000271857 |
| 1:153775217:C:G | rs1453811257 | ENST00000271857 |
| 1:153775220:G:A | rs1333621581 | ENST00000271857 |
| 1:153775221:C:G | | ENST00000271857 |
| 1:153775222:A:T | | ENST00000271857 |
| 1:153775223:G:A | | ENST00000271857 |
| 1:153775223:G:C | | ENST00000271857 |
| 1:153775225:C:G | | ENST00000271857 |
| 1:153775225:C:T | rs1240638844 | ENST00000271857 |
| 1:153775226:C:G | rs760167280 | ENST00000271857 |
| 1:153775226:C:T | | ENST00000271857 |
| 1:153775228:G:A | rs573127426 | ENST00000271857 |
| 1:153775228:G:T | | ENST00000271857 |
| 1:153775229:G:C | | ENST00000271857 |
| 1:153775229:G:A | | ENST00000271857 |
| 1:153775231:G:A | | ENST00000271857 |
| 1:153775232:G:A | | ENST00000271857 |
| 1:153775232:GGA: | | ENST00000271857 |
| G | | |
| 1:153775234:A:AG | | ENST00000271857 |
| 1:153775236:C:G | | ENST00000271857 |
| 1:153775237:G:A | | ENST00000271857 |
| 1:153775237:G:C | rs930474655 | ENST00000271857 |
| 1:153775238:C:T | | ENST00000271857 |
| 1:153775244:G:A | | ENST00000271857 |
| 1:153775246:G:A | | ENST00000271857 |
| 1:153775247:C:T | | ENST00000271857 |
| 1:153775249:G:A | rs1461639779 | ENST00000271857 |
| 1:153775249:G:C | | ENST00000271857 |
| 1:153775250:A:G | | ENST00000271857 |
| 1:153775252:C:T | | ENST00000271857 |
| 1:153775252:C:G | rs750715768 | ENST00000271857 |
| 1:153775253:G:A | | ENST00000271857 |
| 1:153775253:G:T | | ENST00000271857 |
| 1:153775255:A:G | | ENST00000271857 |
| 1:153775259:T:C | | ENST00000271857 |
| 1:153775261:T:TG | rs1184177755 | ENST00000271857 |
| 1:153775261:T:G | | ENST00000271857 |
| 1:153775261:T:C | | ENST00000271857 |
| 1:153775261:TG:T | rs963179258 | ENST00000271857 |
| 1:153775262:G:T | rs369246509 | ENST00000271857 |
| 1:153775262:G:A | rs369246509 | ENST00000271857 |
| 1:153775262:G:C | rs369246509 | ENST00000271857 |
| 1:153775263:G:C | rs540566011 | ENST00000271857 |
| 1:153775263:G:T | rs540566011 | ENST00000271857 |
| 1:153775263:G:A | | ENST00000271857 |
| 1:153775264:G:A | rs565610766 | ENST00000271857 |
| 1:153775264:G:C | rs565610766 | ENST00000271857 |
| 1:153775264:G:T | rs565610766 | ENST00000271857 |
| 1:153775265:G:A | | ENST00000271857 |
| 1:153775265:G:T | rs778911407 | ENST00000271857 |
| 1:153775267:G:T | rs900181370 | ENST00000271857 |
| 1:153775267:G:A | | ENST00000271857 |
| 1:153775267:G:C | | ENST00000271857 |
| 1:153775268:A:G | rs1331085849 | ENST00000271857 |
| 1:153775268:A:T | | ENST00000271857 |
| 1:153775270:G:A | rs755738692 | ENST00000271857 |
| 1:153775271:G:A | rs745589728 | ENST00000271857 |
| 1:153775274:G:C | rs771915237 | ENST00000271857 |
| 1:153775275:G:T | | ENST00000271857 |
| 1:153775276:G:C | | ENST00000271857 |
| 1:153775276:G:T | rs532703647 | ENST00000271857 |
| 1:153775277:A:AG | | ENST00000271857 |
| TCCGGGGCC | | |
| 1:153775277:A:G | | ENST00000271857 |
| 1:153775279:T:A | | ENST00000271857 |
| 1:153775282:G:C | | ENST00000271857 |
| 1:153775285:G:T | | ENST00000271857 |
| 1:153775285:GCCG | | ENST00000271857 |
| CCCCACAC:G | | |
| 1:153775288:G:A | rs1285156476 | ENST00000271857 |
| 1:153775289:C:A | rs1324177781 | ENST00000271857 |
| 1:153775292:C:G | | ENST00000271857 |
| 1:153775292:C:T | | ENST00000271857 |
| 1:153775294:C:T | rs776070776 | ENST00000271857 |
| 1:153775295:AC:A | | ENST00000271857 |
| 1:153775296:C:G | rs761418504 | ENST00000271857 |
| 1:153775297:C:T | rs937469156 | ENST00000271857 |
| 1:153775298:C:T | rs966793831 | ENST00000271857 |
| 1:153775298:C:G | | ENST00000271857 |
| 1:153775298:C:A | rs966793831 | ENST00000271857 |
| 1:153775298:C:CG | | ENST00000271857 |
| CACTCCTCCCGGGT | | |
| TTCT | | |
| 1:153775302:C:A | | ENST00000271857 |
| 1:153775302:C:G | | ENST00000271857 |
| 1:153775303:T:G | | ENST00000271857 |
| 1:153775303:T:C | | ENST00000271857 |
| 1:153775304:C:A | rs763221872 | ENST00000271857 |
| 1:153775304:C:T | | ENST00000271857 |
| 1:153775306:TCC:T | | ENST00000271857 |
| 1:153775307:CCCG | | ENST00000271857 |
| GGTTTCT:C | | |
| 1:153775307:C:T | | ENST00000271857 |
| 1:153775307:C:A | | ENST00000271857 |
| 1:153775309:C:G | rs766888135 | ENST00000271857 |
| 1:153775310:G:A | | ENST00000271857 |
| 1:153775311:GGTT | | ENST00000271857 |
| TCTGC:G | | |
| 1:153775312:GT:G | | ENST00000271857 |
| 1:153775312:G:T | | ENST00000271857 |
| 1:153775312:G:A | rs751808699 | ENST00000271857 |
| 1:153775313:T:A | rs1168183742 | ENST00000271857 |
| 1:153775318:G:C | | ENST00000271857 |
| 1:153775318:G:A | | ENST00000271857 |
| 1:153775319:C:G | | ENST00000271857 |
| 1:153775321:C:T | rs752650677 | ENST00000271857 |
| 1:153775321:C:A | rs752650677 | ENST00000271857 |
| 1:153775322:T:C | rs756228690 | ENST00000271857 |
| 1:153775324:C:G | rs1345531047 | ENST00000271857 |
| 1:153775324:C:T | | ENST00000271857 |
| 1:153775324:C:A | | ENST00000271857 |
| 1:153775325:G:C | | ENST00000271857 |
| 1:153775325:G:T | | ENST00000271857 |
| 1:153775325:G:A | rs367588342 | ENST00000271857 |
| 1:153775328:C:G | | ENST00000271857 |
| 1:153775328:C:T | rs1227847914 | ENST00000271857 |
| 1:153775330:T:A | rs1358982222 | ENST00000271857 |
| 1:153775333:G:A | rs930592262 | ENST00000271857 |
| 1:153775334:G:T | rs746829483 | ENST00000271857 |
| 1:153775334:G:A | rs746829483 | ENST00000271857 |
| 1:153775336:G:C | | ENST00000271857 |
| 1:153775336:G:A | rs754585303 | ENST00000271857 |
| 1:153775337:T:A | rs780903625 | ENST00000271857 |
| 1:153775339:G:C | | ENST00000271857 |
| 1:153775340:T:TG | rs767393433 | ENST00000271857 |
| 1:153775345:G:A | | ENST00000271857 |
| 1:153775345:G:C | rs1260227216 | ENST00000271857 |
| 1:153775345:G:T | | ENST00000271857 |
| 1:153775346:C:T | rs779292108 | ENST00000271857 |
| 1:153775347:C:CT | | ENST00000271857 |
| GGG | | |
| 1:153775348:T:A | rs1427751955 | ENST00000271857 |
| 1:153775351:G:A | rs530584891 | ENST00000271857 |
| 1:153775354:G:A | | ENST00000271857 |
| 1:153775355:G:A | | ENST00000271857 |
| 1:153775357:A:G | rs771587570 | ENST00000368661:ENST00000624995 |
| 1:153775360:G:C | | ENST00000271857:ENST00000368661:ENST00000624995 |
| 1:153775363:G:A | | ENST00000271857:ENST00000368661:ENST00000624995 |
| 1:153775363:G:T | | ENST00000271857:ENST00000368661:ENST00000624995 |
| 1:153775364:T:C | rs963294134 | ENST00000271857:ENST00000368661:ENST00000624995 |
| 1:153775367:G:A | | ENST00000271857:ENST00000368661:ENST00000624995 |
| 1:153775369:C:T | | ENST00000271857:ENST00000368661:ENST00000624995 |
| 1:153775369:C:G | rs775836265 | ENST00000271857:ENST00000368661:ENST00000624995 |
| 1:153775370:A:G | | ENST00000271857:ENST00000368661:ENST00000624995 |
| 1:153775372:C:A | | ENST00000271857:ENST00000368661:ENST00000624995 |
| 1:153775372:C:T | rs1363984329 | ENST00000271857:ENST00000368661:ENST00000624995 |
| 1:153775375:A:G | | ENST00000271857:ENST00000368661:ENST00000624995 |
| 1:153775376:C:T | | ENST00000271857:ENST00000368661:ENST00000624995 |
| 1:153775378:C:T | | ENST00000271857:ENST00000368661:ENST00000624995 |
| 1:153775379:G:A | rs1396809758 | ENST00000271857:ENST00000368661:ENST00000624995 |
| 1:153775381:G:T | rs377764357 | ENST00000271857:ENST00000368661:ENST00000624995 |
| 1:153775385:C:A | rs766335084 | ENST00000271857:ENST00000368661:ENST00000624995 |
| 1:153775385:C:T | rs766335084 | ENST00000271857:ENST00000368661:ENST00000624995 |
| 1:153775387:T:G | rs751289599 | ENST00000271857:ENST00000368661:ENST00000624995 |
| 1:153775387:T:A | rs751289599 | ENST00000271857:ENST00000368661:ENST00000624995 |
| 1:153775387:T:C | | ENST00000271857:ENST00000368661:ENST00000624995 |
| 1:153775388:G:T | | ENST00000271857:ENST00000368661:ENST00000624995 |
| 1:153775388:G:A | | ENST00000271857:ENST00000368661:ENST00000624995 |
| 1:153775389:G:C | | ENST00000271857:ENST00000368661:ENST00000624995 |
| 1:153775389:G:T | | ENST00000271857:ENST00000368661:ENST00000624995 |
| 1:153775390:A:T | | ENST00000271857:ENST00000368661:ENST00000624995 |
| 1:153775392:G:T | | ENST00000271857:ENST00000368661:ENST00000624995 |
| 1:153775393:G:A | rs754783148 | ENST00000271857:ENST00000368661:ENST00000624995 |
| 1:153775395:GAA: | rs1160186120 | ENST00000271857:ENST00000368661:ENST00000624995 |
| G | | |
| 1:153775396:A:G | rs900131274 | ENST00000271857:ENST00000368661:ENST00000624995 |
| 1:153775397:A:C | rs1187553931 | ENST00000271857:ENST00000368661:ENST00000624995 |
| 1:153775398:G:C | | ENST00000271857:ENST00000368661:ENST00000624995 |
| 1:153775400:C:T | | ENST00000271857:ENST00000368661:ENST00000624995 |
| 1:153775402:CAGC | rs1390855453 | ENST00000271857:ENST00000368661:ENST00000624995 |
| T:C | | |
| 1:153775403:A:G | rs780696787 | ENST00000271857:ENST00000368661:ENST00000624995 |
| 1:153775403:A:C | | ENST00000271857:ENST00000368661:ENST00000624995 |
| 1:153775404:G:C | | ENST00000271857:ENST00000368661:ENST00000624995 |
| 1:153775405:C:G | | ENST00000271857:ENST00000368661:ENST00000624995 |
| 1:153775406:T:A | | ENST00000271857:ENST00000368661:ENST00000624995 |
| 1:153775406:T:C | rs747873035 | ENST00000271857:ENST00000368661:ENST00000624995 |
| 1:153775408:G:A | rs1418071462 | ENST00000271857:ENST00000368661:ENST00000624995 |
| 1:153775411:C:T | rs755555852 | ENST00000271857:ENST00000368661:ENST00000624995 |
| 1:153775412:G:A | | ENST00000271857:ENST00000368661:ENST00000624995 |
| 1:153775412:G:T | rs368673134 | ENST00000271857:ENST00000368661:ENST00000624995 |
| 1:153775412:G:C | rs368673134 | ENST00000271857:ENST00000368661:ENST00000624995 |
| 1:153775414:G:C | rs1032706082 | ENST00000271857:ENST00000368661:ENST00000624995 |
| 1:153775414:G:A | rs1032706082 | ENST00000271857:ENST00000368661:ENST00000624995 |
| 1:153775415:C:T | | ENST00000271857:ENST00000368661:ENST00000624995 |
| 1:153775415:C:CG | rs1489501977 | ENST00000271857:ENST00000368661:ENST00000624995 |
| 1:153775415:C:A | rs771495380 | ENST00000271857:ENST00000368661:ENST00000624995 |
| 1:153775418:C:T | rs775008853 | ENST00000271857:ENST00000368661:ENST00000624995 |
| 1:153775420:C:G | | ENST00000271857:ENST00000368661:ENST00000624995 |
| 1:153775421:T:C | | ENST00000271857:ENST00000368661:ENST00000624995 |
| 1:153775423:G:C | | ENST00000271857:ENST00000368661:ENST00000624995 |
| 1:153775423:G:T | | ENST00000271857:ENST00000368661:ENST00000624995 |
| 1:153775423:G:A | rs1214189360 | ENST00000271857:ENST00000368661:ENST00000624995 |
| 1:153775424:G:A | rs1317474919 | ENST00000271857:ENST00000368661:ENST00000624995 |
| 1:153775426:T:A | rs772557420 | ENST00000271857:ENST00000368661:ENST00000624995 |
| 1:153775429:CGGA | rs866877278 | ENST00000271857:ENST00000368661:ENST00000624995 |
| A:C | | |
| 1:153775429:C:T | | ENST00000271857:ENST00000368661:ENST00000624995 |
| 1:153775429:C:G | rs1021091180 | ENST00000271857:ENST00000368661:ENST00000624995 |
| 1:153775429:CGGA | rs866877278 | ENST00000271857:ENST00000368661:ENST00000624995 |
| A:C | | |
| 1:153775430:G:C | | ENST00000271857:ENST00000368661:ENST00000624995 |
| 1:153775430:G:A | rs775546505 | ENST00000271857:ENST00000368661:ENST00000624995 |
| 1:153775430:G:T | | ENST00000271857:ENST00000368661:ENST00000624995 |
| 1:153775432:A:T | | ENST00000271857:ENST00000368661:ENST00000624995 |
| 1:153775432:AAG | rs752830947 | ENST00000271857:ENST00000368661:ENST00000624995 |
| GG:A | | |
| 1:153775433:AG:A | | ENST00000271857:ENST00000368661:ENST00000624995 |
| 1:153775434:G:C | | ENST00000271857:ENST00000368661:ENST00000624995 |
| 1:153775436:G:A | | ENST00000271857:ENST00000368661:ENST00000624995 |
| 1:153775439:G:C | | ENST00000271857:ENST00000368661:ENST00000624995 |
| 1:153775439:G:A | rs764249182 | ENST00000271857:ENST00000368661:ENST00000624995 |
| 1:153775441:T:C | | ENST00000271857:ENST00000368661:ENST00000624995 |
| 1:153775441:T:A | | ENST00000271857:ENST00000368661:ENST00000624995 |
| 1:153775442:C:G | rs1263009445 | ENST00000271857:ENST00000368661:ENST00000624995 |
| 1:153775444:G:A | rs752901679 | ENST00000271857:ENST00000368661:ENST00000624995 |
| 1:153775447:A:G | rs776733663 | ENST00000271857:ENST00000368661:ENST00000624995 |
| 1:153775447:A:T | rs776733663 | ENST00000271857:ENST00000368661:ENST00000624995 |
| 1:153775448:T:C | rs1469322592 | ENST00000271857:ENST00000368661:ENST00000624995 |
| 1:153775449:GT:G | rs1175053090 | ENST00000271857:ENST00000368661:ENST00000624995 |
| 1:153775449:G:A | rs761886426 | ENST00000271857:ENST00000368661:ENST00000624995 |
| 1:153775450:T:C | | ENST00000271857:ENST00000368661:ENST00000624995 |
| 1:153775452:T:G | | ENST00000271857:ENST00000368661:ENST00000624995 |
| 1:153775453:G:A | | ENST00000271857:ENST00000368661:ENST00000624995 |
| 1:153775454:C:G | | ENST00000271857:ENST00000368661:ENST00000624995 |
| 1:153775456:A:T | | ENST00000271857:ENST00000368661:ENST00000624995 |
| 1:153775456:A:G | | ENST00000271857:ENST00000368661:ENST00000624995 |
| 1:153775458:CG:C | | ENST00000271857:ENST00000368661:ENST00000624995 |
| 1:153775459:G:A | rs370290114 | ENST00000271857:ENST00000368661:ENST00000624995 |
| 1:153775462:TG:T | rs756424515 | ENST00000271857:ENST00000368661:ENST00000624995 |
| 1:153775464:G:A | rs1229494073 | ENST00000271857:ENST00000368661:ENST00000624995 |
| 1:153775467:C:G | | ENST00000271857:ENST00000368661:ENST00000624995 |
| 1:153775468:C:A | | ENST00000271857:ENST00000368661:ENST00000624995 |
| 1:153775468:CAG: | | ENST00000271857:ENST00000368661:ENST00000624995 |
| c | | |
| 1:153775471:A:C | | ENST00000271857:ENST00000368661:ENST00000624995 |
| 1:153775471:A:G | | ENST00000271857:ENST00000368661:ENST00000624995 |
| 1:153775472:C:G | | ENST00000271857:ENST00000368661:ENST00000624995 |
| 1:153775472:C:A | | ENST00000271857:ENST00000368661:ENST00000624995 |
| 1:153775472:C:T | | ENST00000271857:ENST00000368661:ENST00000624995 |
| 1:153775475:T:C | | ENST00000271857:ENST00000368661:ENST00000624995 |
| 1:153775478:C:G | rs777291258 | ENST00000271857:ENST00000368661:ENST00000624995 |
| 1:153775481:T:G | | ENST00000271857:ENST00000368661:ENST00000624995 |
| 1:153775481:T:C | | ENST00000271857:ENST00000368661:ENST00000624995 |
| 1:153775484:A:G | | ENST00000271857:ENST00000368661:ENST00000624995 |
| 1:153775485:G:T | | ENST00000271857:ENST00000368661:ENST00000624995 |
| 1:153775486:GA:G | | ENST00000271857:ENST00000368661:ENST00000624995 |
| 1:153775486:G:A | | ENST00000271857:ENST00000368661:ENST00000624995 |
| 1:153775487:A:G | | ENST00000271857:ENST00000368661:ENST00000624995 |
| 1:153775490:C:G | | ENST00000271857:ENST00000368661:ENST00000624995 |
| 1:153775490:C:A | | ENST00000271857:ENST00000368661:ENST00000624995 |
| 1:153775495:T:C | rs756882168 | ENST00000271857:ENST00000368661:ENST00000624995 |
| 1:153775495:T:A | | ENST00000271857:ENST00000368661:ENST00000624995 |
| 1:153775496:C:G | | ENST00000271857:ENST00000368661:ENST00000624995 |
| 1:153775496:C:T | rs1437571822 | ENST00000271857:ENST00000368661:ENST00000624995 |
| 1:153775498:A:G | | ENST00000271857:ENST00000368661:ENST00000624995 |
| 1:153775498:A:C | | ENST00000271857:ENST00000368661:ENST00000624995 |
| 1:153775498:A:T | rs779556521 | ENST00000271857:ENST00000368661:ENST00000624995 |
| 1:153775499:T:C | | ENST00000271857:ENST00000368661:ENST00000624995 |
| 1:153775499:T:TG | | ENST00000271857:ENST00000368661:ENST00000624995 |
| GCTG | | |
| 1:153775499:T:A | | ENST00000271857:ENST00000368661:ENST00000624995 |
| 1:153775500:G:T | rs571482201 | ENST00000271857:ENST00000368661:ENST00000624995 |
| 1:153775501:G:C | | ENST00000271857:ENST00000368661:ENST00000624995 |
| 1:153775502:C:T | rs201459909 | ENST00000271857:ENST00000368661:ENST00000624995 |
| 1:153775502:C:A | | ENST00000271857:ENST00000368661:ENST00000624995 |
| 1:153775504:G:A | | ENST00000271857:ENST00000368661:ENST00000624995 |
| 1:153775504:GC:G | | ENST00000271857:ENST00000368661:ENST00000624995 |
| 1:153775505:C:G | | ENST00000271857:ENST00000368661:ENST00000624995 |
| 1:153775505:C:T | rs145619769 | ENST00000271857:ENST00000368661:ENST00000624995 |
| 1:153775505:C:T | rs145619769 | ENST00000271857:ENST00000368661:ENST00000624995 |
| 1:153775507:C:T | | ENST00000271857:ENST00000368661:ENST00000624995 |
| 1:153775508:T:A | | ENST00000271857:ENST00000368661:ENST00000624995 |
| 1:153775511:T:C | | ENST00000271857:ENST00000368661:ENST00000624995 |
| 1:153775511:T:A | rs747369615 | ENST00000271857:ENST00000368661:ENST00000624995 |
| 1:153775514:T:G | | ENST00000271857:ENST00000368661:ENST00000624995 |
| 1:153775516:C:G | | ENST00000271857:ENST00000368661:ENST00000624995 |
| 1:153775517:T:C | | ENST00000271857:ENST00000368661:ENST00000624995 |
| 1:153775519:C:T | rs149310539 | ENST00000271857:ENST00000368661:ENST00000624995 |
| 1:153775519:C:A | | ENST00000271857:ENST00000368661:ENST00000624995 |
| 1:153775520:C:T | | ENST00000271857:ENST00000368661:ENST00000624995 |
| 1:153775520:C:A | | ENST00000271857:ENST00000368661:ENST00000624995 |
| 1:153775522:C:A | | ENST00000271857:ENST00000368661:ENST00000624995 |
| 1:153775523:T:C | | ENST00000271857:ENST00000368661:ENST00000624995 |
| 1:153775528:C:CT | rs780537591 | ENST00000271857:ENST00000368661:ENST00000624995 |
| 1:153775529:T:C | rs761869783 | ENST00000271857:ENST00000368661:ENST00000624995 |
| 1:153775529:T:G | rs761869783 | ENST00000271857:ENST00000368661:ENST00000624995 |
| 1:153775532:T:C | | ENST00000271857:ENST00000368661:ENST00000624995 |
| 1:153775534:C:A | | ENST00000271857:ENST00000368661:ENST00000624995 |
| 1:153775534:C:G | | ENST00000271857:ENST00000368661:ENST00000624995 |
| 1:153775537:C:T | rs769930407 | ENST00000271857:ENST00000368661:ENST00000624995 |
| 1:153775538:C:G | | ENST00000271857:ENST00000368661:ENST00000624995 |
| 1:153775538:C:T | | ENST00000271857:ENST00000368661:ENST00000624995 |
| 1:153775541:T:A | | ENST00000271857:ENST00000368661:ENST00000624995 |
| 1:153775543:C:G | rs759419064 | ENST00000271857:ENST00000368661:ENST00000624995 |
| 1:153775545:GCTG | | ENST00000271857:ENST00000368661:ENST00000624995 |
| CTGA:G | | |
| 1:153775546:C:G | rs1274756950 | ENST00000271857:ENST00000368661:ENST00000624995 |
| 1:153775547:T:C | | ENST00000271857:ENST00000368661:ENST00000624995 |
| 1:153775549:C:G | | ENST00000271857:ENST00000368661:ENST00000624995 |
| 1:153775550:T:C | | ENST00000271857:ENST00000368661:ENST00000624995 |
| 1:153775553:A:C | rs767385672 | ENST00000271857:ENST00000368661:ENST00000624995 |
| 1:153775553:A:G | | ENST00000271857:ENST00000368661:ENST00000624995 |
| 1:153775555:CTA:C | | ENST00000271857:ENST00000368661:ENST00000624995 |
| 1:153775556:T:C | | ENST00000271857:ENST00000368661:ENST00000624995 |
| 1:153775558:C:T | | ENST00000271857:ENST00000368661:ENST00000624995 |
| 1:153775559:A:G | rs752469654 | ENST00000271857:ENST00000368661:ENST00000624995 |
| 1:153775559:A:T | rs752469654 | ENST00000271857:ENST00000368661:ENST00000624995 |
| 1:153775561:C:T | rs760395879 | ENST00000271857:ENST00000368661:ENST00000624995 |
| 1:153775562:T:G | rs763786463 | ENST00000271857:ENST00000368661:ENST00000624995 |
| 1:153775564:T:C | | ENST00000271857:ENST00000368661:ENST00000624995 |
| 1:153775565:G:A | | ENST00000271857:ENST00000368661:ENST00000624995 |
| 1:153775566:G:A | rs983789638 | ENST00000271857:ENST00000368661:ENST00000624995 |
| 1:153775568:C:T | rs753608758 | ENST00000271857:ENST00000368661:ENST00000624995 |
| 1:153775568:C:G | | ENST00000271857:ENST00000368661:ENST00000624995 |
| 1:153775570:C:T | | ENST00000271857:ENST00000368661:ENST00000624995 |
| 1:153775574:T:TG | | ENST00000271857:ENST00000368661:ENST00000624995 |
| 1:153775576:C:T | rs756798412 | ENST00000271857:ENST00000368661:ENST00000624995 |
| 1:153775576:C:A | | ENST00000271857:ENST00000368661:ENST00000624995 |
| 1:153775577:G:T | | ENST00000271857:ENST00000368661:ENST00000624995 |
| 1:153775580:G:C | | ENST00000271857:ENST00000368661:ENST00000624995 |
| 1:153775580:G:A | rs536324024 | ENST00000271857:ENST00000368661:ENST00000624995 |
| 1:153775580:G:T | rs536324024 | ENST00000271857:ENST00000368661:ENST00000624995 |
| 1:153775589:C:A | | ENST00000271857:ENST00000368661:ENST00000624995 |
| 1:153775593:C:G | | ENST00000271857:ENST00000368661:ENST00000624995 |
| 1:153775593:C:A | | ENST00000271857:ENST00000368661:ENST00000624995 |
| 1:153775595:T:C | | ENST00000271857:ENST00000368661:ENST00000624995 |
| 1:153775595:T:TG | | ENST00000271857:ENST00000368661:ENST00000624995 |
| 1:153775596:G:T | | ENST00000271857:ENST00000368661:ENST00000624995 |
| 1:153775596:G:C | | ENST00000271857:ENST00000368661:ENST00000624995 |
| 1:153775600:T:C | rs780430740 | ENST00000271857:ENST00000368661:ENST00000624995 |
| 1:153775603:G:A | | ENST00000271857:ENST00000368661:ENST00000624995 |
| 1:153775603:G:T | rs747340789 | ENST00000271857:ENST00000368661:ENST00000624995 |
| 1:153775603:G:C | | ENST00000271857:ENST00000368661:ENST00000624995 |
| 1:153775604:C:A | | ENST00000271857:ENST00000368661:ENST00000624995 |
| 1:153775604:C:T | | ENST00000271857:ENST00000368661:ENST00000624995 |
| 1:153775604:C:G | | ENST00000271857:ENST00000368661:ENST00000624995 |
| 1:153775606:G:C | | ENST00000271857:ENST00000368661:ENST00000624995 |
| 1:153775606:G:A | rs950250331 | ENST00000271857:ENST00000368661:ENST00000624995 |
| 1:153775607:T:C | | ENST00000271857:ENST00000368661:ENST00000624995 |
| 1:153775607:T:A | | ENST00000271857:ENST00000368661:ENST00000624995 |
| 1:153775610:G:C | rs1289075234 | ENST00000271857:ENST00000368661:ENST00000624995 |
| 1:153775610:G:A | rs1289075234 | ENST00000271857:ENST00000368661:ENST00000624995 |
| 1:153775612:G:C | | ENST00000271857:ENST00000368661:ENST00000624995 |
| 1:153775612:G:A | rs1361033995 | ENST00000271857:ENST00000368661:ENST00000624995 |
| 1:153775613:C:T | rs1209880499 | ENST00000271857:ENST00000368661:ENST00000624995 |
| 1:153775613:C:G | | ENST00000271857:ENST00000368661:ENST00000624995 |
| 1:153775615:CTG: | rs1445225097 | ENST00000271857:ENST00000368661:ENST00000624995 |
| C | | |
| 1:153775615:C:G | | ENST00000271857:ENST00000368661:ENST00000624995 |
| 1:153775616:T:G | | ENST00000271857:ENST00000368661:ENST00000624995 |
| 1:153775618:T:C | | ENST00000271857:ENST00000368661:ENST00000624995 |
| 1:153775620:C:A | | ENST00000271857:ENST00000368661:ENST00000624995 |
| 1:153775623:CA:C | | ENST00000271857:ENST00000368661:ENST00000624995 |
| 1:153775624:A:G | rs921664529 | ENST00000271857:ENST00000368661:ENST00000624995 |
| 1:153775624:A:C | rs921664529 | ENST00000271857:ENST00000368661:ENST00000624995 |
| 1:153775627:A:T | | ENST00000271857:ENST00000368661:ENST00000624995 |
| 1:153775627:A:G | rs138225868 | ENST00000271857:ENST00000368661:ENST00000624995 |
| 1:153775628:G:C | rs1424226184 | ENST00000271857:ENST00000368661:ENST00000624995 |
| 1:153775628:G:A | | ENST00000271857:ENST00000368661:ENST00000624995 |
| 1:153775629:G:T | | ENST00000271857:ENST00000368661:ENST00000624995 |
| 1:153775631:C:G | | ENST00000271857:ENST00000368661:ENST00000624995 |
| 1:153775633:C:T | rs369929708 | ENST00000271857:ENST00000368661:ENST00000624995 |
| 1:153775636:C:G | rs773235434 | ENST00000271857:ENST00000368661:ENST00000624995 |
| 1:153775637:G:C | rs1280581256 | ENST00000271857:ENST00000368661:ENST00000624995 |
| 1:153775639:G:T | rs762946133 | ENST00000271857:ENST00000368661:ENST00000624995 |
| 1:153775639:G:A | rs762946133 | ENST00000271857:ENST00000368661:ENST00000624995 |
| 1:153775639:G:C | rs762946133 | ENST00000271857:ENST00000368661:ENST00000624995 |
| 1:153775640:C:T | | ENST00000271857:ENST00000368661:ENST00000624995 |
| 1:153775642:C:A | | ENST00000271857:ENST00000368661:ENST00000624995 |
| 1:153775642:C:T | rs777978409 | ENST00000271857:ENST00000368661:ENST00000624995 |
| 1:153775643:G:T | | ENST00000271857:ENST00000368661:ENST00000624995 |
| 1:153775643:G:A | rs892817725 | ENST00000271857:ENST00000368661:ENST00000624995 |
| 1:153775645:G:A | | ENST00000271857:ENST00000368661:ENST00000624995 |
| 1:153775649:T:C | | ENST00000271857:ENST00000368661:ENST00000624995 |
| 1:153775652:C:A | | ENST00000271857:ENST00000368661:ENST00000624995 |
| 1:153775652:C:T | | ENST00000271857:ENST00000368661:ENST00000624995 |
| 1:153775652:CCGC | | ENST00000271857:ENST00000368661:ENST00000624995 |
| GGCTGCCGCCGACC | | |
| CGGAAGGT:C | | |
| 1:153775654:G:A | rs1452344007 | ENST00000271857:ENST00000368661:ENST00000624995 |
| 1:153775654:G:C | | ENST00000271857:ENST00000368661:ENST00000624995 |
| 1:153775655:C:T | | ENST00000271857:ENST00000368661:ENST00000624995 |
| 1:153775657:GC:G | | ENST00000271857:ENST00000368661:ENST00000624995 |
| 1:153775658:C:A | | ENST00000271857:ENST00000368661:ENST00000624995 |
| 1:153775660:GC:G | | ENST00000271857:ENST00000368661:ENST00000624995 |
| 1:153775660:G:A | | ENST00000271857:ENST00000368661:ENST00000624995 |
| 1:153775661:C:A | rs1244993644 | ENST00000271857:ENST00000368661:ENST00000624995 |
| 1:153775663:G:C | | ENST00000271857:ENST00000368661:ENST00000624995 |
| 1:153775664:C:T | rs1347873029 | ENST00000271857:ENST00000368661:ENST00000624995 |
| 1:153775664:C:G | | ENST00000271857:ENST00000368661:ENST00000624995 |
| 1:153775666:G:C | rs1268667788 | ENST00000271857:ENST00000368661:ENST00000624995 |
| 1:153775667:A:T | | ENST00000271857:ENST00000368661:ENST00000624995 |
| 1:153775670:C:A | | ENST00000271857:ENST00000368661:ENST00000624995 |
| 1:153775670:C:T | rs1460738673 | ENST00000271857:ENST00000368661:ENST00000624995 |
| 1:153775670:C:G | | ENST00000271857:ENST00000368661:ENST00000624995 |
| 1:153775672:G:T | | ENST00000271857:ENST00000368661:ENST00000624995 |
| 1:153775672:G:A | | ENST00000271857:ENST00000368661:ENST00000624995 |
| 1:153775672:G:C | rs1398137499 | ENST00000271857:ENST00000368661:ENST00000624995 |
| 1:153775674:AG:A | rs1224900446 | ENST00000271857:ENST00000368661:ENST00000624995 |
| 1:153775675:G:A | rs748759243 | ENST00000271857:ENST00000368661:ENST00000624995 |
| 1:153775675:G:GG | | ENST00000271857:ENST00000368661:ENST00000624995 |
| T | | |
| 1:153775676:G:T | | ENST00000271857:ENST00000368661:ENST00000624995 |
| 1:153775676:G:C | | ENST00000271857:ENST00000368661:ENST00000624995 |
| 1:153775678:C:A | | ENST00000271857:ENST00000368661:ENST00000624995 |
| 1:153775678:C:G | rs764018966 | ENST00000271857:ENST00000368661:ENST00000624995 |
| 1:153775679:C:T | | ENST00000271857:ENST00000368661:ENST00000624995 |
| 1:153775681:G:A | | ENST00000271857:ENST00000368661:ENST00000624995 |
| 1:153775681:G:C | rs1188432101 | ENST00000271857:ENST00000368661:ENST00000624995 |
| 1:153775682:A:G | | ENST00000271857:ENST00000368661:ENST00000624995 |
| 1:153775682:A:T | | ENST00000271857:ENST00000368661:ENST00000624995 |
| 1:153775682:A:AG | rs770447338 | ENST00000271857:ENST00000368661:ENST00000624995 |
| 1:153775683:G:C | rs1244759889 | ENST00000271857:ENST00000368661:ENST00000624995 |
| 1:153775683:G:GT | | ENST00000271857:ENST00000368661:ENST00000624995 |
| 1:153775683:G:T | | ENST00000271857:ENST00000368661:ENST00000624995 |
| 1:153775684:G:T | rs143813756 | ENST00000271857:ENST00000368661:ENST00000624995 |
| 1:153775684:G:A | rs143813756 | ENST00000271857:ENST00000368661:ENST00000624995 |
| 1:153775684:G:C | rs143813756 | ENST00000271857:ENST00000368661:ENST00000624995 |
| 1:153775685:G:T | rs34527123 | ENST00000271857:ENST00000368661:ENST00000624995 |
| 1:153775685:G:C | rs34527123 | ENST00000271857:ENST00000368661:ENST00000624995 |
| 1:153775685:G:A | rs34527123 | ENST00000271857:ENST00000368661:ENST00000624995 |
| 1:153775687:G:A | | ENST00000271857:ENST00000368661:ENST00000624995 |
| 1:153775687:G:T | | ENST00000271857:ENST00000368661:ENST00000624995 |
| 1:153775688:G:GC | | ENST00000271857:ENST00000368661:ENST00000624995 |
| 1:153775688:G:A | rs371171645 | ENST00000271857:ENST00000368661:ENST00000624995 |
| 1:153775688:G:T | rs371171645 | ENST00000271857:ENST00000368661:ENST00000624995 |
| 1:153775690:T:C | | ENST00000271857:ENST00000368661:ENST00000624995 |
| 1:153775691:G:A | | ENST00000271857:ENST00000368661:ENST00000624995 |
| 1:153775692:C:A | | ENST00000271857:ENST00000368661:ENST00000624995 |
| 1:153775693:A:G | | ENST00000271857:ENST00000368661:ENST00000624995 |
| 1:153775694:G:C | rs1408673084 | ENST00000271857:ENST00000368661:ENST00000624995 |
| 1:153775696:C:G | | ENST00000271857:ENST00000368661:ENST00000624995 |
| 1:153775697:T:C | rs1481880301 | ENST00000271857:ENST00000368661:ENST00000624995 |
| 1:153775700:C:A | rs1327708098 | ENST00000271857:ENST00000368661:ENST00000624995 |
| 1:153775702:T:C | | ENST00000271857:ENST00000368661:ENST00000624995 |
| 1:153775704:G:C | | ENST00000271857:ENST00000368661:ENST00000624995 |
| 1:153775704:G:T | | ENST00000271857:ENST00000368661:ENST00000624995 |
| 1:153775706:G:T | | ENST00000271857:ENST00000368661:ENST00000624995 |
| 1:153775708:C:T | rs769765605 | ENST00000271857:ENST00000368661:ENST00000624995 |
| 1:153775712:C:T | | ENST00000271857:ENST00000368661:ENST00000624995 |
| 1:153775712:C:A | rs781486036 | ENST00000271857:ENST00000368661:ENST00000624995 |
| 1:153775715:A:C | | ENST00000271857:ENST00000368661:ENST00000624995 |
| 1:153775717:C:A | rs748540383 | ENST00000271857:ENST00000368661:ENST00000624995 |
| 1:153775718:T:A | | ENST00000271857:ENST00000368661:ENST00000624995 |
| 1:153775720:G:GC | | ENST00000271857:ENST00000368661:ENST00000624995 |
| C | | |
| 1:153775720:G:A | | ENST00000271857:ENST00000368661:ENST00000624995 |
| 1:153775723:C:G | | ENST00000271857:ENST00000368661:ENST00000624995 |
| 1:153775726:C:G | | ENST00000271857:ENST00000368661:ENST00000624995 |
| 1:153775726:C:T | | ENST00000271857:ENST00000368661:ENST00000624995 |
| 1:153775729:C:T | | ENST00000271857:ENST00000368661:ENST00000624995 |
| 1:153775730:G:A | rs756258170 | ENST00000271857:ENST00000368661:ENST00000624995 |
| 1:153775732:G:T | rs199648278 | ENST00000271857:ENST00000368661:ENST00000624995 |
| 1:153775732:G:A | rs199648278 | ENST00000271857:ENST00000368661:ENST00000624995 |
| 1:153775735:G:A | rs1261584788 | ENST00000271857:ENST00000368661:ENST00000624995 |
| 1:153775736:C:T | rs775465840 | ENST00000271857:ENST00000368661:ENST00000624995 |
| 1:153775738:C:A | rs746971043 | ENST00000271857:ENST00000368661:ENST00000624995 |
| 1:153775739:A:G | rs768737853 | ENST00000271857:ENST00000368661:ENST00000624995 |
| 1:153775740:C:A | rs199514238 | ENST00000271857:ENST00000368661:ENST00000624995 |
| 1:153775742:C:G | rs761705819 | ENST00000271857:ENST00000368661:ENST00000624995 |
| 1:153775743:CT:C | | ENST00000271857:ENST00000368661:ENST00000624995 |
| 1:153775744:T:C | rs772801728 | ENST00000271857:ENST00000368661:ENST00000624995 |
| 1:153775744:T:A | rs772801728 | ENST00000271857:ENST00000368661:ENST00000624995 |
| 1:153775744:T:G | | ENST00000271857:ENST00000368661:ENST00000624995 |
| 1:153775747:C:T | rs1392680245 | ENST00000271857:ENST00000368661:ENST00000624995 |
| 1:153775750:A:G | | ENST00000271857:ENST00000368661:ENST00000624995 |
| 1:153775750:A:C | | ENST00000271857:ENST00000368661:ENST00000624995 |
| 1:153775751:T:G | | ENST00000271857:ENST00000368661:ENST00000624995 |
| 1:153775753:C:G | | ENST00000271857:ENST00000368661:ENST00000624995 |
| 1:153775754:A:C | rs765849194 | ENST00000271857:ENST00000368661:ENST00000624995 |
| 1:153775756:G:C | rs751100123 | ENST00000271857:ENST00000368661:ENST00000624995 |
| 1:153775756:G:A | rs751100123 | ENST00000271857:ENST00000368661:ENST00000624995 |
| 1:153775756:G:T | | ENST00000271857:ENST00000368661:ENST00000624995 |
| 1:153775760:C:T | | ENST00000271857:ENST00000368661:ENST00000624995 |
| 1:153775762:C:T | rs756528083 | ENST00000271857:ENST00000368661:ENST00000624995 |
| 1:153775763:G:T | rs200975179 | ENST00000271857:ENST00000368661:ENST00000624995 |
| 1:153775763:G:A | rs200975179 | ENST00000271857:ENST00000368661:ENST00000624995 |
| 1:153775765:C:G | | ENST00000271857:ENST00000368661:ENST00000624995 |
| 1:153775765:C:A | | ENST00000271857:ENST00000368661:ENST00000624995 |
| 1:153775765:C:T | rs757335807 | ENST00000271857:ENST00000368661:ENST00000624995 |
| 1:153775766:G:A | rs139923577 | ENST00000271857:ENST00000368661:ENST00000624995 |
| 1:153775766:G:A | rs139923577 | ENST00000271857:ENST00000368661:ENST00000624995 |
| 1:153775766:G:C | rs139923577 | ENST00000271857:ENST00000368661:ENST00000624995 |
| 1:153775766:G:T | | ENST00000271857:ENST00000368661:ENST00000624995 |
| 1:153775768:T:C | rs747946870 | ENST00000271857:ENST00000368661:ENST00000624995 |
| 1:153775771:A:G | | ENST00000271857:ENST00000368661:ENST00000624995 |
| 1:153775777:T:C | | ENST00000271857:ENST00000368661:ENST00000624995 |
| 1:153775778:C:A | | ENST00000271857:ENST00000368661:ENST00000624995 |
| 1:153775778:CAG: | rs759558879 | ENST00000271857:ENST00000368661:ENST00000624995 |
| C | | |
| 1:153775778:C:CA | | ENST00000271857:ENST00000368661:ENST00000624995 |
| A | | |
| 1:153775780:G:C | | ENST00000271857:ENST00000368661:ENST00000624995 |
| 1:153775780:G:T | | ENST00000271857:ENST00000368661:ENST00000624995 |
| 1:153775781:A:C | | ENST00000271857:ENST00000368661:ENST00000624995 |
| 1:153775782:G:C | | ENST00000271857:ENST00000368661:ENST00000624995 |
| 1:153775783:G:A | rs762565606 | ENST00000271857:ENST00000368661:ENST00000624995 |
| 1:153775786:G:A | | ENST00000271857:ENST00000368661:ENST00000624995 |
| 1:153775789:C:A | | ENST00000271857:ENST00000368661:ENST00000624995 |
| 1:153775790:G:A | rs770644974 | ENST00000271857:ENST00000368661:ENST00000624995 |
| 1:153775790:G:GC | | ENST00000271857:ENST00000368661:ENST00000624995 |
| 1:153775791:CGA: | rs771758102 | ENST00000271857:ENST00000368661:ENST00000624995 |
| C | | |
| 1:153775791:CGA: | rs771758102 | ENST00000271857:ENST00000368661:ENST00000624995 |
| C | | |
| 1:153775792:G:A | | ENST00000271857:ENST00000368661:ENST00000624995 |
| 1:153775793:A:G | rs1168800095 | ENST00000271857:ENST00000368661:ENST00000624995 |
| 1:153775795:A:G | | ENST00000271857:ENST00000368661:ENST00000624995 |
| 1:153775796:G:A | rs561008129 | ENST00000271857:ENST00000368661:ENST00000624995 |
| 1:153775796:G:T | | ENST00000271857:ENST00000368661:ENST00000624995 |
| 1:153775799:A:G | | ENST00000271857:ENST00000368661:ENST00000624995 |
| 1:153775800:CAG: | | ENST00000271857:ENST00000368661:ENST00000624995 |
| C | | |
| 1:153775800:C:A | rs1471512091 | ENST00000271857:ENST00000368661:ENST00000624995 |
| 1:153775800:C:G | | ENST00000271857:ENST00000368661:ENST00000624995 |
| 1:153775802:G:A | rs1422193594 | ENST00000271857:ENST00000368661:ENST00000624995 |
| 1:153775804:G:A | rs1295264239 | ENST00000271857:ENST00000368661:ENST00000624995 |
| 1:153775805:C:T | rs1368211656 | ENST00000271857:ENST00000368661:ENST00000624995 |
| 1:153775805:C:G | | ENST00000271857:ENST00000368661:ENST00000624995 |
| 1:153775809:ACGC | | ENST00000271857:ENST00000368661:ENST00000624995 |
| GCCTTCCTACGTG:A | | |
| 1:153775810:C:T | rs767993919 | ENST00000271857:ENST00000368661:ENST00000624995 |
| 1:153775813:G:A | rs1298578246 | ENST00000271857:ENST00000368661:ENST00000624995 |
| 1:153775816:T:C | | ENST00000271857:ENST00000368661:ENST00000624995 |
| 1:153775822:C:T | rs1287139179 | ENST00000271857:ENST00000368661:ENST00000624995 |
| 1:153775823:G:T | rs760995224 | ENST00000271857:ENST00000368661:ENST00000624995 |
| 1:153775823:G:A | | ENST00000271857:ENST00000368661:ENST00000624995 |
| 1:153775825:G:T | | ENST00000271857:ENST00000368661:ENST00000624995 |
| 1:153775826:C:A | rs958724280 | ENST00000271857:ENST00000368661:ENST00000624995 |
| 1:153775826:C:T | | ENST00000271857:ENST00000368661:ENST00000624995 |
| 1:153775826:C:G | | ENST00000271857:ENST00000368661:ENST00000624995 |
| 1:153775828:C:G | | ENST00000271857:ENST00000368661:ENST00000624995 |
| 1:153775832:G:A | | ENST00000271857:ENST00000368661:ENST00000624995 |
| 1:153775834:T:C | | ENST00000271857:ENST00000368661:ENST00000624995 |
| 1:153775835:G:GT | | ENST00000271857:ENST00000368661:ENST00000624995 |
| CCCCGAAACCAA | | |
| 1:153775835:G:GT | | ENST00000271857:ENST00000368661:ENST00000624995 |
| CCCCGAAACCAAGG | | |
| CAGA | | |
| 1:153775837:G:GC | | ENST00000271857:ENST00000368661:ENST00000624995 |
| AGAGGAAGGC | | |
| 1:153775837:G:A | | ENST00000271857:ENST00000368661:ENST00000624995 |
| 1:153775838:A:AA | | ENST00000271857:ENST00000368661:ENST00000624995 |
| GGCAGGGGT | | |
| 1:153775838:A:G | rs1376057074 | ENST00000271857:ENST00000368661:ENST00000624995 |
| 1:153775839:C:G | | ENST00000271857:ENST00000368661:ENST00000624995 |
| 1:153775840:T:A | rs1267811146 | ENST00000271857:ENST00000368661:ENST00000624995 |
| 1:153775841:G:A | | ENST00000271857:ENST00000368661:ENST00000624995 |
| 1:153775842:G:C | | ENST00000271857:ENST00000368661:ENST00000624995 |
| 1:153775843:G:A | rs764414485 | ENST00000271857:ENST00000368661:ENST00000624995 |
| 1:153775846:C:G | | ENST00000271857:ENST00000368661:ENST00000624995 |
| 1:153775847:C:A | | ENST00000271857:ENST00000368661:ENST00000624995 |
| 1:153775849:G:A | rs200003433 | ENST00000271857:ENST00000368661:ENST00000624995 |
| 1:153775850:A:C | | ENST00000271857:ENST00000368661:ENST00000624995 |
| 1:153775851:C:A | | ENST00000271857:ENST00000368661:ENST00000624995 |
| 1:153775851:C:G | | ENST00000271857:ENST00000368661:ENST00000624995 |
| 1:153775852:G:A | rs750412690 | ENST00000271857:ENST00000368661:ENST00000624995 |
| 1:153775856:G:A | rs758612487 | ENST00000271857:ENST00000368661:ENST00000624995 |
| 1:153775858:G:A | rs546727237 | ENST00000271857:ENST00000368661:ENST00000624995 |
| 1:153775861:A:G | rs781275017 | ENST00000271857:ENST00000368661:ENST00000624995 |
| 1:153775862:G:C | | ENST00000271857:ENST00000368661:ENST00000624995 |
| 1:153775864:G:T | rs748140586 | ENST00000271857:ENST00000368661:ENST00000624995 |
| 1:153775864:G:A | | ENST00000271857:ENST00000368661:ENST00000624995 |
| 1:153775865:G:A | rs1312373320 | ENST00000271857:ENST00000368661:ENST00000624995 |
| 1:153775868:A:AG | | ENST00000271857:ENST00000368661:ENST00000624995 |
| 1:153775870:G:C | | ENST00000271857:ENST00000368661:ENST00000624995 |
| 1:153775873:A:C | | ENST00000271857:ENST00000368661:ENST00000624995 |
| 1:153775874:G:C | | ENST00000271857:ENST00000368661:ENST00000624995 |
| 1:153775875:C:A | rs749111000 | ENST00000271857:ENST00000368661:ENST00000624995 |
| 1:153775876:G:A | rs770429395 | ENST00000271857:ENST00000368661:ENST00000624995 |
| 1:153775877:C:T | | ENST00000271857:ENST00000368661:ENST00000624995 |
| 1:153775879:G:A | | ENST00000271857:ENST00000368661:ENST00000624995 |
| 1:153775880:G:A | rs774107164 | ENST00000271857:ENST00000368661:ENST00000624995 |
| 1:153775883:A:G | | ENST00000271857:ENST00000368661:ENST00000624995 |
| 1:153775885:G:A | | ENST00000271857:ENST00000368661:ENST00000624995 |
| 1:153775888:G:C | | ENST00000271857:ENST00000368661:ENST00000624995 |
| 1:153775888:G:T | rs950223186 | ENST00000271857:ENST00000368661:ENST00000624995 |
| 1:153775888:G:A | rs950223186 | ENST00000271857:ENST00000368661:ENST00000624995 |
| 1:153775889:A:G | | ENST00000271857:ENST00000368661:ENST00000624995 |
| 1:153775890:G:T | | ENST00000271857:ENST00000368661:ENST00000624995 |
| 1:153775891:C:T | | ENST00000271857:ENST00000368661:ENST00000624995 |
| 1:153775891:C:G | | ENST00000271857:ENST00000368661:ENST00000624995 |
| 1:153775892:G:T | rs1239090630 | ENST00000271857:ENST00000368661:ENST00000624995 |
| 1:153775894:G:A | rs1047677868 | ENST00000271857:ENST00000368661:ENST00000624995 |
| 1:153775898:C:G | | ENST00000271857:ENST00000368661:ENST00000624995 |
| 1:153775898:C:T | | ENST00000271857:ENST00000368661:ENST00000624995 |
| 1:153775900:C:T | rs1459712406 | ENST00000271857:ENST00000368661:ENST00000624995 |
| 1:153775901:C:A | | ENST00000271857:ENST00000368661:ENST00000624995 |
| 1:153775901:C:T | | ENST00000271857:ENST00000368661:ENST00000624995 |
| 1:153775904:GAGC | | ENST00000271857:ENST00000368661:ENST00000624995 |
| CGGAGATGCAGCG | | |
| GCCGGA:G | | |
| 1:153775904:G:T | rs559005134 | ENST00000271857:ENST00000368661:ENST00000624995 |
| 1:153775904:G:A | rs559005134 | ENST00000271857:ENST00000368661:ENST00000624995 |
| 1:153775906:GC:G | | ENST00000271857:ENST00000368661:ENST00000624995 |
| 1:153775907:C:T | | ENST00000271857:ENST00000368661:ENST00000624995 |
| 1:153775907:C:G | | ENST00000271857:ENST00000368661:ENST00000624995 |
| 1:153775909:G:A | | ENST00000271857:ENST00000368661:ENST00000624995 |
| 1:153775912:G:A | | ENST00000271857:ENST00000368661:ENST00000624995 |
| 1:153775913:A:G | rs764695214 | ENST00000271857:ENST00000368661:ENST00000624995 |
| 1:153775913:A:T | rs764695214 | ENST00000271857:ENST00000368661:ENST00000624995 |
| 1:153775919:C:T | rs1468572471 | ENST00000271857:ENST00000368661:ENST00000624995 |
| 1:153775919:C:A | | ENST00000271857:ENST00000368661:ENST00000624995 |
| 1:153775919:C:G | | ENST00000271857:ENST00000368661:ENST00000624995 |
| 1:153775921:G:A | rs765365021 | ENST00000271857:ENST00000368661:ENST00000624995 |
| 1:153775922:C:G | | ENST00000271857:ENST00000368661:ENST00000624995 |
| 1:153775924:G:A | rs758525342 | ENST00000271857:ENST00000368661:ENST00000624995 |
| 1:153775924:G:T | | ENST00000271857:ENST00000368661:ENST00000624995 |
| 1:153775925:G:A | rs780334151 | ENST00000271857:ENST00000368661:ENST00000624995 |
| 1:153775928:G:A | | ENST00000271857:ENST00000368661:ENST00000624995 |
| 1:153775928:G:C | | ENST00000271857:ENST00000368661:ENST00000624995 |
| 1:153775930:G:A | rs751714680 | ENST00000271857:ENST00000368661:ENST00000624995 |
| 1:153775931:G:A | | ENST00000271857:ENST00000368661:ENST00000624995 |
| 1:153775933:G:A | | ENST00000271857:ENST00000368661:ENST00000624995 |
| 1:153775934:C:T | rs755936017 | ENST00000271857:ENST00000368661:ENST00000624995 |
| 1:153775936:G:A | rs777777588 | ENST00000271857:ENST00000368661:ENST00000624995 |
| 1:153775937:A:G | rs1331826133 | ENST00000271857:ENST00000368661:ENST00000624995 |
| 1:153775939:T:C | rs1041415643 | ENST00000271857:ENST00000368661:ENST00000624995 |
| 1:153775940:T:C | | ENST00000271857:ENST00000368661:ENST00000624995 |
| 1:153775941:T:G | | ENST00000271857:ENST00000368661:ENST00000624995 |
| 1:153775942:G:C | | ENST00000271857:ENST00000368661:ENST00000624995 |
| 1:153775943:C:T | | ENST00000271857:ENST00000368661:ENST00000624995 |
| 1:153775945:G:A | | ENST00000271857:ENST00000368661:ENST00000624995 |
| 1:153775945:G:C | rs1182150857 | ENST00000271857:ENST00000368661:ENST00000624995 |
| 1:153775945:G:T | | ENST00000271857:ENST00000368661:ENST00000624995 |
| 1:153775946:GA:G | | ENST00000271857:ENST00000368661:ENST00000624995 |
| 1:153775946:G:C | | ENST00000271857:ENST00000368661:ENST00000624995 |
| 1:153775948:G:A | rs890378411 | ENST00000271857:ENST00000368661:ENST00000624995 |
| 1:153775949:G:C | | ENST00000271857:ENST00000368661:ENST00000624995 |
| 1:153775949:G:A | rs749019368 | ENST00000271857:ENST00000368661:ENST00000624995 |
| 1:153775949:G:T | | ENST00000271857:ENST00000368661:ENST00000624995 |
| 1:153775951:G:A | rs1321808745 | ENST00000271857:ENST00000368661:ENST00000624995 |
| 1:153775951:G:C | | ENST00000271857:ENST00000368661:ENST00000624995 |
| 1:153775951:GA:G | | ENST00000271857:ENST00000368661:ENST00000624995 |
| 1:153775952:A:T | | ENST00000271857:ENST00000368661:ENST00000624995 |
| 1:153775952:A:G | | ENST00000271857:ENST00000368661:ENST00000624995 |
| 1:153775953:C:A | rs370083818 | ENST00000271857:ENST00000368661:ENST00000624995 |
| 1:153775954:G:A | rs778399497 | ENST00000271857:ENST00000368661:ENST00000624995 |
| 1:153775955:G:A | rs1181406286 | ENST00000271857:ENST00000368661:ENST00000624995 |
| 1:153775955:G:C | rs1181406286 | ENST00000271857:ENST00000368661:ENST00000624995 |
| 1:153775958:C:T | | ENST00000271857:ENST00000368661:ENST00000624995 |
| 1:153775960:G:A | rs771733267 | ENST00000271857:ENST00000368661:ENST00000624995 |
| 1:153775963:A:G | | ENST00000271857:ENST00000368661:ENST00000624995 |
| 1:153775963:A:T | | ENST00000271857:ENST00000368661:ENST00000624995 |
| 1:153775964:G:C | | ENST00000271857:ENST00000368661:ENST00000624995 |
| 1:153775965:A:T | rs140781371 | ENST00000271857:ENST00000368661:ENST00000624995 |
| 1:153775966:G:C | rs774939595 | ENST00000271857:ENST00000368661:ENST00000624995 |
| 1:153775966:G:A | | ENST00000271857:ENST00000368661:ENST00000624995 |
| 1:153775967:G:A | rs746527312 | ENST00000271857:ENST00000368661:ENST00000624995 |
| 1:153775969:G:A | rs1246883931 | ENST00000271857:ENST00000368661:ENST00000624995 |
| 1:153775970:G:C | | ENST00000271857:ENST00000368661:ENST00000624995 |
| 1:153775970:G:A | | ENST00000271857:ENST00000368661:ENST00000624995 |
| 1:153775972:G:A | | ENST00000271857:ENST00000368661:ENST00000624995 |
| 1:153775973:G:A | | ENST00000271857:ENST00000368661:ENST00000624995 |
| 1:153775975:G:C | | ENST00000271857:ENST00000368661:ENST00000624995 |
| 1:153775975:G:A | | ENST00000271857:ENST00000368661:ENST00000624995 |
| 1:153775976:C:A | | ENST00000271857:ENST00000368661:ENST00000624995 |
| 1:153775978:G:T | rs762083552 | ENST00000271857:ENST00000368661:ENST00000624995 |
| 1:153775978:G:A | rs762083552 | ENST00000271857:ENST00000368661:ENST00000624995 |
| 1:153775981:G:T | rs773549207 | ENST00000271857:ENST00000368661:ENST00000624995 |
| 1:153775981:G:C | rs773549207 | ENST00000271857:ENST00000368661:ENST00000624995 |
| 1:153775984:CCT:C | | ENST00000271857:ENST00000368661:ENST00000624995 |
| 1:153775984:C:T | | ENST00000271857:ENST00000368661:ENST00000624995 |
| 1:153775985:C:A | | ENST00000271857:ENST00000368661:ENST00000624995 |
| 1:153775985:C:T | | ENST00000271857:ENST00000368661:ENST00000624995 |
| 1:153775985:C:G | | ENST00000271857:ENST00000368661:ENST00000624995 |
| 1:153775988:T:C | | ENST00000271857:ENST00000368661:ENST00000624995 |
| 1:153775990:T:C | rs766710897 | ENST00000271857:ENST00000368661:ENST00000624995 |
| 1:153775991:C:T | rs751628904 | ENST00000271857:ENST00000368661:ENST00000624995 |
| 1:153775993:C:T | | ENST00000271857:ENST00000368661:ENST00000624995 |
| 1:153775993:C:G | | ENST00000271857:ENST00000368661:ENST00000624995 |
| 1:153775996:G:A | | ENST00000271857:ENST00000368661:ENST00000624995 |
| 1:153775997:G:T | | ENST00000271857:ENST00000368661:ENST00000624995 |
| 1:153775997:G:C | | ENST00000271857:ENST00000368661:ENST00000624995 |
| 1:153776000:C:T | rs1352342488 | ENST00000271857:ENST00000368661:ENST00000624995 |
| 1:153776002:A:G | | ENST00000271857:ENST00000368661:ENST00000624995 |
| 1:153776003:C:A | | ENST00000271857:ENST00000368661:ENST00000624995 |
| 1:153776005:G:A | rs763929708 | ENST00000271857:ENST00000368661:ENST00000624995 |
| 1:153776006:T:C | rs753854112 | ENST00000271857:ENST00000368661:ENST00000624995 |
| 1:153776008:G:C | | ENST00000271857:ENST00000368661:ENST00000624995 |
| 1:153776012:T:C | | ENST00000271857:ENST00000368661:ENST00000624995 |
| 1:153776015:T:A | | ENST00000271857:ENST00000368661:ENST00000624995 |
| 1:153776015:T:G | | ENST00000271857:ENST00000368661:ENST00000624995 |
| 1:153776015:T:C | rs756978807 | ENST00000271857:ENST00000368661:ENST00000624995 |
| 1:153776017:C:T | rs778629923 | ENST00000271857:ENST00000368661:ENST00000624995 |
| 1:153776020:C:T | | ENST00000271857:ENST00000368661:ENST00000624995 |
| 1:153776021:C:T | rs1306573157 | ENST00000271857:ENST00000368661:ENST00000624995 |
| 1:153776021:C:A | | ENST00000271857:ENST00000368661:ENST00000624995 |
| 1:153776021:C:G | | ENST00000271857:ENST00000368661:ENST00000624995 |
| 1:153776023:G:C | rs745691798 | ENST00000271857:ENST00000368661:ENST00000624995 |
| 1:153776023:G:A | | ENST00000271857:ENST00000368661:ENST00000624995 |
| 1:153776023:G:T | | ENST00000271857:ENST00000368661:ENST00000624995 |
| 1:153776024:C:T | rs1295518868 | ENST00000271857:ENST00000368661:ENST00000624995 |
| 1:153776026:G:A | | ENST00000271857:ENST00000368661:ENST00000624995 |
| 1:153776027:G:T | | ENST00000271857:ENST00000368661:ENST00000624995 |
| 1:153776027:G:A | | ENST00000271857:ENST00000368661:ENST00000624995 |
| 1:153776027:GC:G | | ENST00000271857:ENST00000368661:ENST00000624995 |
| 1:153776030:C:T | rs1198671124 | ENST00000271857:ENST00000368661:ENST00000624995 |
| 1:153776030:CAG: | | ENST00000271857:ENST00000368661:ENST00000624995 |
| C | | |
| 1:153776034:G:C | rs1305925792 | ENST00000271857:ENST00000368661:ENST00000624995 |
| 1:153776035:T:C | | ENST00000271857:ENST00000368661:ENST00000624995 |
| 1:153776038:C:G | rs1027700179 | ENST00000271857:ENST00000368661:ENST00000624995 |
| 1:153776044:C:T | | ENST00000271857:ENST00000368661:ENST00000624995 |
| 1:153776048:G:A | rs757980775 | ENST00000271857:ENST00000368661:ENST00000624995 |
| 1:153776048:G:T | | ENST00000271857:ENST00000368661:ENST00000624995 |
| 1:153776048:G:C | rs757980775 | ENST00000271857:ENST00000368661:ENST00000624995 |
| 1:153776053:G:C | rs1409615052 | ENST00000271857:ENST00000368661:ENST00000624995 |
| 1:153776053:G:A | | ENST00000271857:ENST00000368661:ENST00000624995 |
| 1:153776054:G:A | rs768279028 | ENST00000271857:ENST00000368661:ENST00000624995 |
| 1:153776057:T:G | rs748742242 | ENST00000271857:ENST00000368661:ENST00000624995 |
| 1:153776059:G:C | | ENST00000271857:ENST00000368661:ENST00000624995 |
| 1:153776062:A:T | | ENST00000271857:ENST00000368661:ENST00000624995 |
| 1:153776062:A:AA | | ENST00000271857:ENST00000368661:ENST00000624995 |
| GGCCGGCCT | | |
| 1:153776062:A:C | | ENST00000271857:ENST00000368661:ENST00000624995 |
| 1:153776063:A:G | rs139357302 | ENST00000271857:ENST00000368661:ENST00000624995 |
| 1:153776064:G:C | rs770229230 | ENST00000271857:ENST00000368661:ENST00000624995 |
| 1:153776065:G:C | | ENST00000271857:ENST00000368661:ENST00000624995 |
| 1:153776066:C:G | | ENST00000271857:ENST00000368661:ENST00000624995 |
| 1:153776066:C:A | | ENST00000271857:ENST00000368661:ENST00000624995 |
| 1:153776068:G:A | | ENST00000271857:ENST00000368661:ENST00000624995 |
| 1:153776069:G:C | rs773674131 | ENST00000271857:ENST00000368661:ENST00000624995 |
| 1:153776071:C:G | rs528841427 | ENST00000271857:ENST00000368661:ENST00000624995 |
| 1:153776072:T:C | rs1195427635 | ENST00000271857:ENST00000368661:ENST00000624995 |
| 1:153776072:T:G | | ENST00000271857:ENST00000368661:ENST00000624995 |
| 1:153776074:C:T | | ENST00000271857:ENST00000368661:ENST00000624995 |
| 1:153776077:A:C | rs567885989 | ENST00000271857:ENST00000368661:ENST00000624995 |
| 1:153776078:C:A | | ENST00000271857:ENST00000368661:ENST00000624995 |
| 1:153776080:G:C | rs767683594 | ENST00000271857:ENST00000368661:ENST00000624995 |
| 1:153776080:G:A | rs767683594 | ENST00000271857:ENST00000368661:ENST00000624995 |
| 1:153776081:C:T | | ENST00000271857:ENST00000368661:ENST00000624995 |
| 1:153776084:T:A | | ENST00000271857:ENST00000368661:ENST00000624995 |
| 1:153776085:T:A | | ENST00000271857:ENST00000368661:ENST00000624995 |
| 1:153776086:G:C | | ENST00000271857:ENST00000368661:ENST00000624995 |
| 1:153776089:C:G | | ENST00000271857:ENST00000368661:ENST00000624995 |
| 1:153776089:C:T | rs150046345 | ENST00000271857:ENST00000368661:ENST00000624995 |
| 1:153776090:C:A | rs1186313389 | ENST00000271857:ENST00000368661:ENST00000624995 |
| 1:153776090:C:T | | ENST00000271857:ENST00000368661:ENST00000624995 |
| 1:153776092:A:C | rs1222133015 | ENST00000271857:ENST00000368661:ENST00000624995 |
| 1:153776093:C:T | | ENST00000271857:ENST00000368661:ENST00000624995 |
| 1:153776095:G:T | | ENST00000271857:ENST00000368661:ENST00000624995 |
| 1:153776096:C:T | rs1474276364 | ENST00000271857:ENST00000368661:ENST00000624995 |
| 1:153776096:C:G | | ENST00000271857:ENST00000368661:ENST00000624995 |
| 1:153776099:T:C | rs765162301 | ENST00000271857:ENST00000368661:ENST00000624995 |
| 1:153776101:C:A | | ENST00000271857:ENST00000368661:ENST00000624995 |
| 1:153776101:C:T | | ENST00000271857:ENST00000368661:ENST00000624995 |
| 1:153776102:G:T | | ENST00000271857:ENST00000368661:ENST00000624995 |
| 1:153776102:G:A | rs1163020592 | ENST00000271857:ENST00000368661:ENST00000624995 |
| 1:153776104:C:T | | ENST00000271857:ENST00000368661:ENST00000624995 |
| 1:153776105:G:C | | ENST00000271857:ENST00000368661:ENST00000624995 |
| 1:153776105:G:A | rs758248300 | ENST00000271857:ENST00000368661:ENST00000624995 |
| 1:153776107:G:T | rs972064986 | ENST00000271857:ENST00000368661:ENST00000624995 |
| 1:153776107:G:C | | ENST00000271857:ENST00000368661:ENST00000624995 |
| 1:153776108:G:GC | | ENST00000271857:ENST00000368661:ENST00000624995 |
| 1:153776108:G:C | | ENST00000271857:ENST00000368661:ENST00000624995 |
| 1:153776108:G:A | rs779677418 | ENST00000271857:ENST00000368661:ENST00000624995 |
| 1:153776108:GC:G | | ENST00000271857:ENST00000368661:ENST00000624995 |
| 1:153776110:C:G | rs768369879 | ENST00000271857:ENST00000368661:ENST00000624995 |
| 1:153776110:C:T | rs768369879 | ENST00000271857:ENST00000368661:ENST00000624995 |
| 1:153776111:C:G | | ENST00000271857:ENST00000368661:ENST00000624995 |
| 1:153776111:C:T | | ENST00000271857:ENST00000368661:ENST00000624995 |
| 1:153776114:T:G | | ENST00000271857:ENST00000368661:ENST00000624995 |
| 1:153776114:T:C | | ENST00000271857:ENST00000368661:ENST00000624995 |
| 1:153776117:T:C | | ENST00000271857:ENST00000368661:ENST00000624995 |
| 1:153776119:C:T | rs1357804003 | ENST00000271857:ENST00000368661:ENST00000624995 |
| 1:153776120:A:C | rs550497581 | ENST00000271857:ENST00000368661:ENST00000624995 |
| 1:153776120:A:G | rs550497581 | ENST00000271857:ENST00000368661:ENST00000624995 |
| 1:153776122:T:A | | ENST00000271857:ENST00000368661:ENST00000624995 |
| 1:153776123:G:A | rs771458827 | ENST00000271857:ENST00000368661:ENST00000624995 |
| 1:153776123:G:C | | ENST00000271857:ENST00000368661:ENST00000624995 |
| 1:153776124:C:G | rs1463429586 | ENST00000271857:ENST00000368661:ENST00000624995 |
| 1:153776125:C:G | rs774020233 | ENST00000271857:ENST00000368661:ENST00000624995 |
| 1:153776126:T:G | rs774691917 | ENST00000271857:ENST00000368661:ENST00000624995 |
| 1:153776127:CCGC | rs1420417862 | ENST00000271857:ENST00000368661:ENST00000624995 |
| AGCTGCGGCGCGC | | |
| G:C | | |
| 1:153776128:C:T | rs772213803 | ENST00000271857:ENST00000368661:ENST00000624995 |
| 1:153776131:A:G | | ENST00000271857:ENST00000368661:ENST00000624995 |
| 1:153776132:G:C | | ENST00000271857:ENST00000368661:ENST00000624995 |
| 1:153776133:C:A | | ENST00000271857:ENST00000368661:ENST00000624995 |
| 1:153776134:TG:T | | ENST00000271857:ENST00000368661:ENST00000624995 |
| 1:153776136:C:G | | ENST00000271857:ENST00000368661:ENST00000624995 |
| 1:153776137:G:GG | rs761963446 | ENST00000271857:ENST00000368661:ENST00000624995 |
| CGC | | |
| 1:153776137:G:GG | | ENST00000271857:ENST00000368661:ENST00000624995 |
| A | | |
| 1:153776137:G:A | | ENST00000271857:ENST00000368661:ENST00000624995 |
| 1:153776137:GGC: | rs754136629 | ENST00000271857:ENST00000368661:ENST00000624995 |
| G | | |
| 1:153776137:G:GG | rs761963446 | ENST00000271857:ENST00000368661:ENST00000624995 |
| C | | |
| 1:153776140:G:T | rs914315752 | ENST00000271857:ENST00000368661:ENST00000624995 |
| 1:153776140:G:A | | ENST00000271857:ENST00000368661:ENST00000624995 |
| 1:153776141:C:T | rs1164379409 | ENST00000271857:ENST00000368661:ENST00000624995 |
| 1:153776144:G:C | rs1407892136 | ENST00000271857:ENST00000368661:ENST00000624995 |
| 1:153776144:G:A | rs1407892136 | ENST00000271857:ENST00000368661:ENST00000624995 |
| 1:153776146:G:A | | ENST00000271857:ENST00000368661:ENST00000624995 |
| 1:153776147:C:G | | ENST00000271857:ENST00000368661:ENST00000624995 |
| 1:153776147:C:T | rs577404258 | ENST00000271857:ENST00000368661:ENST00000624995 |
| 1:153776149:C:A | rs1161845494 | ENST00000271857:ENST00000368661:ENST00000624995 |
| 1:153776150:T:A | | ENST00000271857:ENST00000368661:ENST00000624995 |
| 1:153776150:T:C | rs1295097050 | ENST00000271857:ENST00000368661:ENST00000624995 |
| 1:153776152:G:C | | ENST00000271857:ENST00000368661:ENST00000624995 |
| 1:153776152:G:A | rs1451503251 | ENST00000271857:ENST00000368661:ENST00000624995 |
| 1:153776152:G:T | rs1451503251 | ENST00000271857:ENST00000368661:ENST00000624995 |
| 1:153776153:T:G | | ENST00000271857:ENST00000368661:ENST00000624995 |
| 1:153776153:T:C | | ENST00000271857:ENST00000368661:ENST00000624995 |
| 1:153776155:C:G | | ENST00000271857:ENST00000368661:ENST00000624995 |
| 1:153776156:T:C | | ENST00000271857:ENST00000368661:ENST00000624995 |
| 1:153776158:G:A | | ENST00000271857:ENST00000368661:ENST00000624995 |
| 1:153776159:C:T | | ENST00000271857:ENST00000368661:ENST00000624995 |
| 1:153776160:G:GC | | ENST00000271857:ENST00000368661:ENST00000624995 |
| 1:153776162:C:A | | ENST00000271857:ENST00000368661:ENST00000624995 |
| 1:153776163:AG:A | | ENST00000271857:ENST00000368661:ENST00000624995 |
| 1:153776164:G:A | | ENST00000271857:ENST00000368661:ENST00000624995 |
| 1:153776164:G:C | | ENST00000271857:ENST00000368661:ENST00000624995 |
| 1:153776165:G:A | | ENST00000271857:ENST00000368661:ENST00000624995 |
| 1:153776165:G:C | | ENST00000271857:ENST00000368661:ENST00000624995 |
| 1:153776166:T:C | rs750427502 | ENST00000271857:ENST00000368661:ENST00000624995 |
| 1:153776516:A:T | | ENST00000271857:ENST00000368661:ENST00000624995 |
| 1:153776517:G:C | rs1201187174 | ENST00000271857:ENST00000368661:ENST00000624995 |
| 1:153776517:G:T | | ENST00000271857:ENST00000368661:ENST00000624995 |
| 1:153776519:GT:G | | ENST00000271857:ENST00000368661:ENST00000624995 |
| 1:153776527:A:G | | ENST00000271857:ENST00000368661:ENST00000624995 |
| 1:153776529:TC:T | | ENST00000271857:ENST00000368661:ENST00000624995 |
| 1:153776530:C:T | | ENST00000271857:ENST00000368661:ENST00000624995 |
| 1:153776533:T:A | rs760741581 | ENST00000271857:ENST00000368661:ENST00000624995 |
| 1:153776535:G:A | | ENST00000271857:ENST00000368661:ENST00000624995 |
| 1:153776535:G:C | | ENST00000271857:ENST00000368661:ENST00000624995 |
| 1:153776538:C:T | | ENST00000271857:ENST00000368661:ENST00000624995 |
| 1:153776539:C:T | rs768764740 | ENST00000271857:ENST00000368661:ENST00000624995 |
| 1:153776541:G:C | | ENST00000271857:ENST00000368661:ENST00000624995 |
| 1:153776541:G:T | rs762832221 | ENST00000271857:ENST00000368661:ENST00000624995 |
| 1:153776542:A:C | | ENST00000271857:ENST00000368661:ENST00000624995 |
| 1:153776543:C:A | | ENST00000271857:ENST00000368661:ENST00000624995 |
| 1:153776545:T:G | | ENST00000271857:ENST00000368661:ENST00000624995 |
| 1:153776545:T:C | rs751304493 | ENST00000271857:ENST00000368661:ENST00000624995 |
| 1:153776547:C:T | | ENST00000271857:ENST00000368661:ENST00000624995 |
| 1:153776548:C:G | rs759304178 | ENST00000271857:ENST00000368661:ENST00000624995 |
| 1:153776550:G:T | rs752328083 | ENST00000271857:ENST00000368661:ENST00000624995 |
| 1:153776550:G:A | | ENST00000271857:ENST00000368661:ENST00000624995 |
| 1:153776551:C:G | | ENST00000271857:ENST00000368661:ENST00000624995 |
| 1:153776551:C:A | | ENST00000271857:ENST00000368661:ENST00000624995 |
| 1:153776551:C:T | | ENST00000271857:ENST00000368661:ENST00000624995 |
| 1:153776554:TGA: | rs1308629613 | ENST00000271857:ENST00000368661:ENST00000624995 |
| T | | |
| 1:153776557:G:T | | ENST00000271857:ENST00000368661:ENST00000624995 |
| 1:153776557:G:A | rs1050445494 | ENST00000271857:ENST00000368661:ENST00000624995 |
| 1:153776558:A:C | | ENST00000271857:ENST00000368661:ENST00000624995 |
| 1:153776561:CAT:C | | ENST00000271857:ENST00000368661:ENST00000624995 |
| 1:153776562:A:G | rs777368122 | ENST00000271857:ENST00000368661:ENST00000624995 |
| 1:153776563:T:C | | ENST00000271857:ENST00000368661:ENST00000624995 |
| 1:153776563:T:TG | | ENST00000271857:ENST00000368661:ENST00000624995 |
| 1:153776565:G:A | rs1435497544 | ENST00000271857:ENST00000368661:ENST00000624995 |
| 1:153776566:G:A | | ENST00000271857:ENST00000368661:ENST00000624995 |
| 1:153776568:C:T | | ENST00000271857:ENST00000368661:ENST00000624995 |
| 1:153776569:T:A | | ENST00000271857:ENST00000368661:ENST00000624995 |
| 1:153776569:T:G | | ENST00000271857:ENST00000368661:ENST00000624995 |
| 1:153776572:A:C | | ENST00000271857:ENST00000368661:ENST00000624995 |
| 1:153776572:A:G | rs779299625 | ENST00000271857:ENST00000368661:ENST00000624995 |
| 1:153776573:C:A | | ENST00000271857:ENST00000368661:ENST00000624995 |
| 1:153776574:C:A | | ENST00000271857:ENST00000368661:ENST00000624995 |
| 1:153776575:T:A | | ENST00000271857:ENST00000368661:ENST00000624995 |
| 1:153776578:G:A | | ENST00000271857:ENST00000368661:ENST00000624995 |
| 1:153776581:C:T | | ENST00000271857:ENST00000368661:ENST00000624995 |
| 1:153776583:G:T | rs746232099 | ENST00000271857:ENST00000368661:ENST00000624995 |
| 1:153776584:C:T | rs758758310 | ENST00000271857:ENST00000368661:ENST00000624995 |
| 1:153776586:G:C | rs35212289 | ENST00000271857:ENST00000368661:ENST00000624995 |
| 1:153776587:G:A | rs1235505455 | ENST00000271857:ENST00000368661:ENST00000624995 |
| 1:153776589:C:G | | ENST00000271857:ENST00000368661:ENST00000624995 |
| 1:153776589:C:T | rs201631543 | ENST00000271857:ENST00000368661:ENST00000624995 |
| 1:153776590:C:G | | ENST00000271857:ENST00000368661:ENST00000624995 |
| 1:153776592:G:A | | ENST00000271857:ENST00000368661:ENST00000624995 |
| 1:153776593:G:A | | ENST00000271857:ENST00000368661:ENST00000624995 |
| 1:153776596:C:T | rs776656622 | ENST00000271857:ENST00000368661:ENST00000624995 |
| 1:153776598:C:T | | ENST00000271857:ENST00000368661:ENST00000624995 |
| 1:153776599:A:C | | ENST00000271857:ENST00000368661:ENST00000624995 |
| 1:153776599:A:G | rs963000739 | ENST00000271857:ENST00000368661:ENST00000624995 |
| 1:153776600:C:G | | ENST00000271857:ENST00000368661:ENST00000624995 |
| 1:153776601:C:T | | ENST00000271857:ENST00000368661:ENST00000624995 |
| 1:153776602:C:T | | ENST00000271857:ENST00000368661:ENST00000624995 |
| 1:153776604:G:T | | ENST00000271857:ENST00000368661:ENST00000624995 |
| 1:153776607:G:A | | ENST00000271857:ENST00000368661:ENST00000624995 |
| 1:153776608:G:C | | ENST00000271857:ENST00000368661:ENST00000624995 |
| 1:153776610:A:G | | ENST00000271857:ENST00000368661:ENST00000624995 |
| 1:153776613:A:G | | ENST00000271857:ENST00000368661:ENST00000624995 |
| 1:153776614:G:C | | ENST00000271857:ENST00000368661:ENST00000624995 |
| 1:153776616:G:T | | ENST00000271857:ENST00000368661:ENST00000624995 |
| 1:153776616:G:C | rs770847119 | ENST00000271857:ENST00000368661:ENST00000624995 |
| 1:153776616:G:A | rs770847119 | ENST00000271857:ENST00000368661:ENST00000624995 |
| 1:153776617:AT:A | | ENST00000271857:ENST00000368661:ENST00000624995 |
| 1:153776619:TTGC | rs750891790 | ENST00000271857:ENST00000368661:ENST00000624995 |
| TGGCTGAAGTGTC: | | |
| T | | |
| 1:153776628:G:T | | ENST00000271857:ENST00000368661:ENST00000624995 |
| 1:153776632:T:C | rs759342432 | ENST00000271857:ENST00000368661:ENST00000624995 |
| 1:153776632:T:G | | ENST00000271857:ENST00000368661:ENST00000624995 |
| 1:153776634:T:C | rs1320221120 | ENST00000271857:ENST00000368661:ENST00000624995 |
| 1:153776635:C:T | | ENST00000271857:ENST00000368661:ENST00000624995 |
| 1:153776637:G:C | rs149254183 | ENST00000271857:ENST00000368661:ENST00000624995 |
| 1:153776637:G:T | | ENST00000271857:ENST00000368661:ENST00000624995 |
| 1:153776637:G:A | rs149254183 | ENST00000271857:ENST00000368661:ENST00000624995 |
| 1:153776641:A:C | | ENST00000271857:ENST00000368661:ENST00000624995 |
| 1:153776647:A:G | | ENST00000271857:ENST00000368661:ENST00000624995 |
| 1:153776648:T:G | | ENST00000271857:ENST00000368661:ENST00000624995 |
| 1:153776649:G:A | | ENST00000271857:ENST00000368661:ENST00000624995 |
| 1:153776652:C:T | | ENST00000271857:ENST00000368661:ENST00000624995 |
| 1:153776653:C:CA | rs750807379 | ENST00000271857:ENST00000368661:ENST00000624995 |
| 1:153776656:T:C | | ENST00000271857:ENST00000368661:ENST00000624995 |
| 1:153776658:C:G | | ENST00000271857:ENST00000368661:ENST00000624995 |
| 1:153776658:C:T | | ENST00000271857:ENST00000368661:ENST00000624995 |
| 1:153776658:C:A | | ENST00000271857:ENST00000368661:ENST00000624995 |
| 1:153776659:C:A | rs753338879 | ENST00000271857:ENST00000368661:ENST00000624995 |
| 1:153776659:C:T | rs753338879 | ENST00000271857:ENST00000368661:ENST00000624995 |
| 1:153776659:C:G | | ENST00000271857:ENST00000368661:ENST00000624995 |
| 1:153776662:G:A | rs1418310002 | ENST00000271857:ENST00000368661:ENST00000624995 |
| 1:153776663:A:AT | rs1364389700 | ENST00000271857:ENST00000368661:ENST00000624995 |
| ACC | | |
| 1:153776665:A:G | | ENST00000271857:ENST00000368661:ENST00000624995 |
| 1:153776666:CCT:C | | ENST00000271857:ENST00000368661:ENST00000624995 |
| 1:153776667:C:T | rs1028491356 | ENST00000271857:ENST00000368661:ENST00000624995 |
| 1:153776668:T:A | | ENST00000271857:ENST00000368661:ENST00000624995 |
| 1:153776668:T:C | rs1267719108 | ENST00000271857:ENST00000368661:ENST00000624995 |
| 1:153776673:TC:T | rs751881128 | ENST00000271857:ENST00000368661:ENST00000624995 |
| 1:153776673:T:A | | ENST00000271857:ENST00000368661:ENST00000624995 |
| 1:153776673T:TC | rs780443002 | ENST00000271857:ENST00000368661:ENST00000624995 |
| 1:153776674:C:T | rs1208478605 | ENST00000271857:ENST00000368661:ENST00000624995 |
| 1:153776674:C:A | | ENST00000271857:ENST00000368661:ENST00000624995 |
| 1:153776674:C:G | | ENST00000271857:ENST00000368661:ENST00000624995 |
| 1:153776676:C:T | rs765771008 | ENST00000271857:ENST00000368661:ENST00000624995 |
| 1:153776676:C:A | | ENST00000271857:ENST00000368661:ENST00000624995 |
| 1:153776677:C:T | rs539968144 | ENST00000271857:ENST00000368661:ENST00000624995 |
| 1:153776679:C:A | | ENST00000271857:ENST00000368661:ENST00000624995 |
| 1:153776679:C:T | rs780236527 | ENST00000271857:ENST00000368661:ENST00000624995 |
| 1:153776679:C:G | | ENST00000271857:ENST00000368661:ENST00000624995 |
| 1:153776680:A:C | | ENST00000271857:ENST00000368661:ENST00000624995 |
| 1:153776682:A:G | | ENST00000271857:ENST00000368661:ENST00000624995 |
| 1:153776682:A:T | | ENST00000271857:ENST00000368661:ENST00000624995 |
| 1:153776683:G:A | rs998972394 | ENST00000271857:ENST00000368661:ENST00000624995 |
| 1:153776686:T:A | | ENST00000271857:ENST00000368661:ENST00000624995 |
| 1:153776687:A:G | | ENST00000271857:ENST00000368661:ENST00000624995 |
| 1:153776688:A:G | rs200391365 | ENST00000271857:ENST00000368661:ENST00000624995 |
| 1:153776693:C:CA | rs777354007 | ENST00000271857:ENST00000368661:ENST00000624995 |
| 1:153776694:A:T | rs1456564512 | ENST00000271857:ENST00000368661:ENST00000624995 |
| 1:153776695:C:T | rs1156688706 | ENST00000271857:ENST00000368661:ENST00000624995 |
| 1:153776698:G:A | rs747409588 | ENST00000271857:ENST00000368661:ENST00000624995 |
| 1:153776698:G:T | rs747409588 | ENST00000271857:ENST00000368661:ENST00000624995 |
| 1:153776698:G:C | rs747409588 | ENST00000271857:ENST00000368661:ENST00000624995 |
| 1:153776701:T:A | rs565076905 | ENST00000271857:ENST00000368661:ENST00000624995 |
| 1:153776701:T:C | rs565076905 | ENST00000271857:ENST00000368661:ENST00000624995 |
| 1:153776703:T:C | rs376383074 | ENST00000271857:ENST00000368661:ENST00000624995 |
| 1:153776704:A:G | rs1378583180 | ENST00000271857:ENST00000368661:ENST00000624995 |
| 1:153776706:A:G | | ENST00000271857:ENST00000368661:ENST00000624995 |
| 1:153776708:C:G | rs769801335 | ENST00000271857:ENST00000368661:ENST00000624995 |
| 1:153776712:A:G | | ENST00000271857:ENST00000368661:ENST00000624995 |
| 1:153776713:C:T | rs745739373 | ENST00000271857:ENST00000368661:ENST00000624995 |
| 1:153776715:T:C | rs1322862865 | ENST00000271857:ENST00000368661:ENST00000624995 |
| 1:153776716:C:G | | ENST00000271857:ENST00000368661:ENST00000624995 |
| 1:153776719:G:A | | ENST00000271857:ENST00000368661:ENST00000624995 |
| 1:153776721:AC:A | rs985863093 | ENST00000271857:ENST00000368661:ENST00000624995 |
| 1:153776724:A:G | rs911659848 | ENST00000271857:ENST00000368661:ENST00000624995 |
| 1:153776725:C:T | rs1228251261 | ENST00000271857:ENST00000368661:ENST00000624995 |
| 1:153776727:G:A | rs959816883 | ENST00000271857:ENST00000368661:ENST00000624995 |
| 1:153776727:G:T | | ENST00000271857:ENST00000368661:ENST00000624995 |
| 1:153776728:G:A | | ENST00000271857:ENST00000368661:ENST00000624995 |
| 1:153777060:A:C | | ENST00000271857:ENST00000368661:ENST00000624995 |
| 1:153777061:G:T | | ENST00000271857:ENST00000368661:ENST00000624995 |
| 1:153777062:G:A | | ENST00000271857:ENST00000368661:ENST00000624995 |
| 1:153777062:G:T | rs140283520 | ENST00000271857:ENST00000368661:ENST00000624995 |
| 1:153777068:C:G | | ENST00000271857:ENST00000368661:ENST00000624995 |
| 1:153777069:CA:C | rs1376196704 | ENST00000271857:ENST00000368661:ENST00000624995 |
| 1:153777071:A:G | rs762542422 | ENST00000271857:ENST00000368661:ENST00000624995 |
| 1:153777073:G:A | | ENST00000271857:ENST00000368661:ENST00000624995 |
| 1:153777074:C:G | | ENST00000271857:ENST00000368661:ENST00000624995 |
| 1:153777074:C:T | | ENST00000271857:ENST00000368661:ENST00000624995 |
| 1:153777077:C:T | rs1392613479 | ENST00000271857:ENST00000368661:ENST00000624995 |
| 1:153777077:C:G | | ENST00000271857:ENST00000368661:ENST00000624995 |
| 1:153777079:C:T | rs150357360 | ENST00000271857:ENST00000368661:ENST00000624995 |
| 1:153777079:C:T | rs150357360 | ENST00000271857:ENST00000368661:ENST00000624995 |
| 1:153777079:C:G | rs150357360 | ENST00000271857:ENST00000368661:ENST00000624995 |
| 1:153777080:G:C | rs138178771 | ENST00000271857:ENST00000368661:ENST00000624995 |
| 1:153777080:G:A | rs138178771 | ENST00000271857:ENST00000368661:ENST00000624995 |
| 1:153777081:GATC | rs1397081768 | ENST00000271857:ENST00000368661:ENST00000624995 |
| A:G | | |
| 1:153777084:C:G | rs756222831 | ENST00000271857:ENST00000368661:ENST00000624995 |
| 1:153777085:A:G | | ENST00000271857:ENST00000368661:ENST00000624995 |
| 1:153777085:A:C | rs1303651369 | ENST00000271857:ENST00000368661:ENST00000624995 |
| 1:153777086:G:A | | ENST00000271857:ENST00000368661:ENST00000624995 |
| 1:153777088:C:T | | ENST00000271857:ENST00000368661:ENST00000624995 |
| 1:153777088:C:A | | ENST00000271857:ENST00000368661:ENST00000624995 |
| 1:153777089:A:T | rs778067257 | ENST00000271857:ENST00000368661:ENST00000624995 |
| 1:153777094:A:G | | ENST00000271857:ENST00000368661:ENST00000624995 |
| 1:153777097:A:G | | ENST00000271857:ENST00000368661:ENST00000624995 |
| 1:153777098:T:C | | ENST00000271857:ENST00000368661:ENST00000624995 |
| 1:153777098:T:A | rs367975823 | ENST00000271857:ENST00000368661:ENST00000624995 |
| 1:153777100:C:G | rs375378070 | ENST00000271857:ENST00000368661:ENST00000624995 |
| 1:153777104:A:G | rs888229091 | ENST00000271857:ENST00000368661:ENST00000624995 |
| 1:153777107:G:A | | ENST00000271857:ENST00000368661:ENST00000624995 |
| 1:153777107:G:C | | ENST00000271857:ENST00000368661:ENST00000624995 |
| 1:153777111:G:C | | ENST00000271857:ENST00000368661:ENST00000624995 |
| 1:153777112:G:A | | ENST00000271857:ENST00000368661:ENST00000624995 |
| 1:153777113:G:A | | ENST00000271857:ENST00000368661:ENST00000624995 |
| 1:153777115:T:C | rs1377278376 | ENST00000271857:ENST00000368661:ENST00000624995 |
| 1:153777116:T:TA | | ENST00000271857:ENST00000368661:ENST00000624995 |
| 1:153777119:A:G | rs768739520 | ENST00000271857:ENST00000368661:ENST00000624995 |
| 1:153777121:C:T | | ENST00000271857:ENST00000368661:ENST00000624995 |
| 1:153777121:C:G | | ENST00000271857:ENST00000368661:ENST00000624995 |
| 1:153777124:CT:C | | ENST00000271857:ENST00000368661:ENST00000624995 |
| 1:153777125:T:C | | ENST00000271857:ENST00000368661:ENST00000624995 |
| 1:153777131:G:A | rs769274892 | ENST00000271857:ENST00000368661:ENST00000624995 |
| 1:153777133:G:A | | ENST00000271857:ENST00000368661:ENST00000624995 |
| 1:153777136:C:G | | ENST00000271857:ENST00000368661:ENST00000624995 |
| 1:153777137:A:G | | ENST00000271857:ENST00000368661:ENST00000624995 |
| 1:153777137:A:C | | ENST00000271857:ENST00000368661:ENST00000624995 |
| 1:153777139:C:T | | ENST00000271857:ENST00000368661:ENST00000624995 |
| 1:153777142:G:A | | ENST00000271857:ENST00000368661:ENST00000624995 |
| 1:153777146:A:G | | ENST00000271857:ENST00000368661:ENST00000624995 |
| 1:153777148:G:A | | ENST00000271857:ENST00000368661:ENST00000624995 |
| 1:153777150:G:GA | rs1285014691 | ENST00000271857:ENST00000368661:ENST00000624995 |
| 1:153777152:T:C | | ENST00000271857:ENST00000368661:ENST00000624995 |
| 1:153777152:T:G | rs1392032251 | ENST00000271857:ENST00000368661:ENST00000624995 |
| 1:153777154:TA:T | rs1316459862 | ENST00000271857:ENST00000368661:ENST00000624995 |
| 1:153777155:A:C | | ENST00000271857:ENST00000368661:ENST00000624995 |
| 1:153777157:C:G | | ENST00000271857:ENST00000368661:ENST00000624995 |
| 1:153777158:T:C | rs770474020 | ENST00000271857:ENST00000368661:ENST00000624995 |
| 1:153777160:G:A | rs759993538 | ENST00000271857:ENST00000368661:ENST00000624995 |
| 1:153777160:G:T | rs759993538 | ENST00000271857:ENST00000368661:ENST00000624995 |
| 1:153777160:G:C | | ENST00000271857:ENST00000368661:ENST00000624995 |
| 1:153777161:C:T | rs1364021444 | ENST00000271857:ENST00000368661:ENST00000624995 |
| 1:153777163:C:A | | ENST00000271857:ENST00000368661:ENST00000624995 |
| 1:153777164:T:C | | ENST00000271857:ENST00000368661:ENST00000624995 |
| 1:153777166:C:T | rs747842753 | ENST00000271857:ENST00000368661:ENST00000624995 |
| 1:153777167:C:T | | ENST00000271857:ENST00000368661:ENST00000624995 |
| 1:153777169:C:G | | ENST00000271857:ENST00000368661:ENST00000624995 |
| 1:153777169:C:T | | ENST00000271857:ENST00000368661:ENST00000624995 |
| 1:153777170:T:G | rs1324843069 | ENST00000271857:ENST00000368661:ENST00000624995 |
| 1:153777172:T:C | | ENST00000271857:ENST00000368661:ENST00000624995 |
| 1:153777173:A:G | rs761157110 | ENST00000271857:ENST00000368661:ENST00000624995 |
| 1:153777175:C:T | | ENST00000271857:ENST00000368661:ENST00000624995 |
| 1:153777176:A:G | rs1216572377 | ENST00000271857:ENST00000368661:ENST00000624995 |
| 1:153777176:A:T | | ENST00000271857:ENST00000368661:ENST00000624995 |
| 1:153777178:A:G | rs143746183 | ENST00000271857:ENST00000368661:ENST00000624995 |
| 1:153777178:A:T | rs143746183 | ENST00000271857:ENST00000368661:ENST00000624995 |
| 1:153777180:G:A | | ENST00000271857:ENST00000368661:ENST00000624995 |
| 1:153777181:T:A | | ENST00000271857:ENST00000368661:ENST00000624995 |
| 1:153777181:T:C | | ENST00000271857:ENST00000368661:ENST00000624995 |
| 1:153777182:C:A | rs1183982767 | ENST00000271857:ENST00000368661:ENST00000624995 |
| 1:153777184:G:C | rs200519940 | ENST00000271857:ENST00000368661:ENST00000624995 |
| 1:153777184:G:A | rs200519940 | ENST00000271857:ENST00000368661:ENST00000624995 |
| 1:153777185:G:A | | ENST00000271857:ENST00000368661:ENST00000624995 |
| 1:153777188:C:T | | ENST00000271857:ENST00000368661:ENST00000624995 |
| 1:153777193:C:A | rs746544937 | ENST00000271857:ENST00000368661:ENST00000624995 |
| 1:153777194:T:G | rs754898570 | ENST00000271857:ENST00000368661:ENST00000624995 |
| 1:153777199:A:T | | ENST00000271857:ENST00000368661:ENST00000624995 |
| 1:153777202:G:A | rs770282287 | ENST00000271857:ENST00000368661:ENST00000624995 |
| 1:153777203:TG:T | | ENST00000271857:ENST00000368661:ENST00000624995 |
| 1:153777205:G:A | | ENST00000271857:ENST00000368661:ENST00000624995 |
| 1:153777206:G:A | | ENST00000271857:ENST00000368661:ENST00000624995 |
| 1:153777210:C:CAT | rs1399682803 | ENST00000271857:ENST00000368661:ENST00000624995 |
| 1:153777211:A:G | rs747886391 | ENST00000271857:ENST00000368661:ENST00000624995 |
| 1:153777212:T:G | | ENST00000271857:ENST00000368661:ENST00000624995 |
| 1:153777215:G:A | | ENST00000271857:ENST00000368661:ENST00000624995 |
| 1:153777217:A:G | rs756007300 | ENST00000271857:ENST00000368661:ENST00000624995 |
| 1:153777220:G:T | | ENST00000271857:ENST00000368661:ENST00000624995 |
| 1:153777221:G:C | | ENST00000271857:ENST00000368661:ENST00000624995 |
| 1:153777222:T:C | | ENST00000271857:ENST00000368661:ENST00000624995 |
| 1:153777759:A:G | rs750124826 | ENST00000271857:ENST00000368661:ENST00000624995 |
| 1:153777760:G:A | | ENST00000271857:ENST00000368661:ENST00000624995 |
| 1:153777761:G:C | | ENST00000271857:ENST00000368661:ENST00000624995 |
| 1:153777763:G:GA | | ENST00000271857:ENST00000368661:ENST00000624995 |
| 1:153777763:G:A | rs758010177 | ENST00000271857:ENST00000368661:ENST00000624995 |
| 1:153777763:G:C | | ENST00000271857:ENST00000368661:ENST00000624995 |
| 1:153777764:C:T | rs139575560 | ENST00000271857:ENST00000368661:ENST00000624995 |
| 1:153777766:A:G | | ENST00000271857:ENST00000368661:ENST00000624995 |
| 1:153777767:C:T | | ENST00000271857:ENST00000368661:ENST00000624995 |
| 1:153777769:G:C | | ENST00000271857:ENST00000368661:ENST00000624995 |
| 1:153777769:G:GT | rs745343614 | ENST00000271857:ENST00000368661:ENST00000624995 |
| 1:153777769:G:GT | rs745343614 | ENST00000271857:ENST00000368661:ENST00000624995 |
| 1:153777770:T:C | | ENST00000271857:ENST00000368661:ENST00000624995 |
| 1:153777772:G:A | | ENST00000271857:ENST00000368661:ENST00000624995 |
| 1:153777772:G:C | | ENST00000271857:ENST00000368661:ENST00000624995 |
| 1:153777776:T:A | rs1032164165 | ENST00000271857:ENST00000368661:ENST00000624995 |
| 1:153777779:A:T | | ENST00000271857:ENST00000368661:ENST00000624995 |
| 1:153777780:A:C | rs1458912560 | ENST00000271857:ENST00000368661:ENST00000624995 |
| 1:153777781:T:G | rs769149759 | ENST00000271857:ENST00000368661:ENST00000624995 |
| 1:153777781:TC:T | rs746960985 | ENST00000271857:ENST00000368661:ENST00000624995 |
| 1:153777781:T:TC | rs780021407 | ENST00000271857:ENST00000368661:ENST00000624995 |
| 1:153777785:A:G | rs781611111 | ENST00000271857:ENST00000368661:ENST00000624995 |
| 1:153777785:A:C | | ENST00000271857:ENST00000368661:ENST00000624995 |
| 1:153777789:C:A | rs748521231 | ENST00000271857:ENST00000368661:ENST00000624995 |
| 1:153777791:C:T | rs199984591 | ENST00000271857:ENST00000368661:ENST00000624995 |
| 1:153777791:C:T | rs199984591 | ENST00000271857:ENST00000368661:ENST00000624995 |
| 1:153777793:G:A | rs771059232 | ENST00000271857:ENST00000368661:ENST00000624995 |
| 1:153777793:G:T | | ENST00000271857:ENST00000368661:ENST00000624995 |
| 1:153777794:C:T | | ENST00000271857:ENST00000368661:ENST00000624995 |
| 1:153777796:G:A | | ENST00000271857:ENST00000368661:ENST00000624995 |
| 1:153777799:C:G | rs759590227 | ENST00000271857:ENST00000368661:ENST00000624995 |
| 1:153777799:C:T | rs759590227 | ENST00000271857:ENST00000368661:ENST00000624995 |
| 1:153777801:GT:G | rs768570076 | ENST00000271857:ENST00000368661:ENST00000624995 |
| 1:153777804:C:G | | ENST00000271857:ENST00000368661:ENST00000624995 |
| 1:153777805:T:C | rs764086117 | ENST00000271857:ENST00000368661:ENST00000624995 |
| 1:153777811:G:T | | ENST00000271857:ENST00000368661:ENST00000624995 |
| 1:153777812:A:G | rs761573364 | ENST00000271857:ENST00000368661:ENST00000624995 |
| 1:153777812:A:T | | ENST00000271857:ENST00000368661:ENST00000624995 |
| 1:153777814:T:C | | ENST00000271857:ENST00000368661:ENST00000624995 |
| 1:153777814:T:A | | ENST00000271857:ENST00000368661:ENST00000624995 |
| 1:153777815:G:T | | ENST00000271857:ENST00000368661:ENST00000624995 |
| 1:153777816:C:G | | ENST00000271857:ENST00000368661:ENST00000624995 |
| 1:153777821:A:T | | ENST00000271857:ENST00000368661:ENST00000624995 |
| 1:153777825:C:G | | ENST00000271857:ENST00000368661:ENST00000624995 |
| 1:153777826:A:G | rs1233105925 | ENST00000271857:ENST00000368661:ENST00000624995 |
| 1:153777826:AGG: | | ENST00000271857:ENST00000368661:ENST00000624995 |
| A | | |
| 1:153777830:T:C | rs758002673 | ENST00000271857:ENST00000368661:ENST00000624995 |
| 1:153777830:T:G | rs758002673 | ENST00000271857:ENST00000368661:ENST00000624995 |
| 1:153777832:A:T | rs779588083 | ENST00000271857:ENST00000368661:ENST00000624995 |
| 1:153777833:C:T | rs151230291 | ENST00000271857:ENST00000368661:ENST00000624995 |
| 1:153777833:C:G | | ENST00000271857:ENST00000368661:ENST00000624995 |
| 1:153777839:T:G | rs1389798378 | ENST00000271857:ENST00000368661:ENST00000624995 |
| 1:153777840:C:A | rs781756523 | ENST00000271857:ENST00000368661:ENST00000624995 |
| 1:153777841:C:T | rs141304230 | ENST00000271857:ENST00000368661:ENST00000624995 |
| 1:153777841:C:T | rs141304230 | ENST00000271857:ENST00000368661:ENST00000624995 |
| 1:153777842:A:G | | ENST00000271857:ENST00000368661:ENST00000624995 |
| 1:153777845:AC:A | | ENST00000271857:ENST00000368661:ENST00000624995 |
| 1:153777847:A:T | rs374690026 | ENST00000271857:ENST00000368661:ENST00000624995 |
| 1:153777847:A:G | rs374690026 | ENST00000271857:ENST00000368661:ENST00000624995 |
| 1:153777848:T:C | rs146814975 | ENST00000271857:ENST00000368661:ENST00000624995 |
| 1:153777848:T:C | rs146814975 | ENST00000271857:ENST00000368661:ENST00000624995 |
| 1:153777849:T:G | | ENST00000271857:ENST00000368661:ENST00000624995 |
| 1:153777850:G:A | | ENST00000271857:ENST00000368661:ENST00000624995 |
| 1:153777850:G:T | | ENST00000271857:ENST00000368661:ENST00000624995 |
| 1:153777851:G:C | | ENST00000271857:ENST00000368661:ENST00000624995 |
| 1:153777851:G:A | rs1340082690 | ENST00000271857:ENST00000368661:ENST00000624995 |
| 1:153777853:G:A | rs1230325283 | ENST00000271857:ENST00000368661:ENST00000624995 |
| 1:153777859:T:G | | ENST00000271857:ENST00000368661:ENST00000624995 |
| 1:153777860:G:A | rs1400495108 | ENST00000271857:ENST00000368661:ENST00000624995 |
| 1:153777862:C:T | rs201878467 | ENST00000271857:ENST00000368661:ENST00000624995 |
| 1:153777862:C:T | rs201878467 | ENST00000271857:ENST00000368661:ENST00000624995 |
| 1:153777863:G:C | | ENST00000271857:ENST00000368661:ENST00000624995 |
| 1:153777863:G:A | rs77673307 | ENST00000271857:ENST00000368661:ENST00000624995 |
| 1:153777863:G:A | rs77673307 | ENST00000271857:ENST00000368661:ENST00000624995 |
| 1:153777867:C:G | | ENST00000271857:ENST00000368661:ENST00000624995 |
| 1:153777867:C:A | | ENST00000271857:ENST00000368661:ENST00000624995 |
| 1:153777868:C:T | rs775569610 | ENST00000271857:ENST00000368661:ENST00000624995 |
| 1:153777869:T:C | rs761700019 | ENST00000271857:ENST00000368661:ENST00000624995 |
| 1:153777871:G:A | rs1241723583 | ENST00000271857:ENST00000368661:ENST00000624995 |
| 1:153777871:G:C | | ENST00000271857:ENST00000368661:ENST00000624995 |
| 1:153777872:T:C | | ENST00000271857:ENST00000368661:ENST00000624995 |
| 1:153777872:T:G | | ENST00000271857:ENST00000368661:ENST00000624995 |
| 1:153777872:TCAA | | ENST00000271857:ENST00000368661:ENST00000624995 |
| C:T | | |
| 1:153777874:A:T | | ENST00000271857:ENST00000368661:ENST00000624995 |
| 1:153777875:AC:A | rs776627744 | ENST00000271857:ENST00000368661:ENST00000624995 |
| 1:153777876:C:G | | ENST00000271857:ENST00000368661:ENST00000624995 |
| 1:153777879:GC:G | rs1245719944 | ENST00000271857:ENST00000368661:ENST00000624995 |
| 1:153777879:G:GC | | ENST00000271857:ENST00000368661:ENST00000624995 |
| 1:153777880:C:G | | ENST00000271857:ENST00000368661:ENST00000624995 |
| 1:153777880:C:T | | ENST00000271857:ENST00000368661:ENST00000624995 |
| 1:153777881:C:T | rs747928166 | ENST00000271857:ENST00000368661:ENST00000624995 |
| 1:153777881:C:A | | ENST00000271857:ENST00000368661:ENST00000624995 |
| 1:153777883:C:T | rs772935114 | ENST00000271857:ENST00000368661:ENST00000624995 |
| 1:153777884:C:A | | ENST00000271857:ENST00000368661:ENST00000624995 |
| 1:153777884:C:T | rs146297812 | ENST00000271857:ENST00000368661:ENST00000624995 |
| 1:153777884:C:T | rs146297812 | ENST00000271857:ENST00000368661:ENST00000624995 |
| 1:153777886:G:C | rs376715519 | ENST00000271857:ENST00000368661:ENST00000624995 |
| 1:153777887:T:C | | ENST00000271857:ENST00000368661:ENST00000624995 |
| 1:153778158:CAG: | rs1421281344 | ENST00000271857:ENST00000368661:ENST00000624995 |
| C | | |
| 1:153778160:G:C | | ENST00000271857:ENST00000368661:ENST00000624995 |
| 1:153778160:G:T | | ENST00000271857:ENST00000368661:ENST00000624995 |
| 1:153778165:A:G | | ENST00000271857:ENST00000368661:ENST00000624995 |
| 1:153778165:A:C | | ENST00000271857:ENST00000368661:ENST00000624995 |
| 1:153778167:G:A | | ENST00000271857:ENST00000368661:ENST00000624995 |
| 1:153778167:G:C | rs1254929682 | ENST00000271857:ENST00000368661:ENST00000624995 |
| 1:153778168:C:T | | ENST00000271857:ENST00000368661:ENST00000624995 |
| 1:153778173:C:G | | ENST00000271857:ENST00000368661:ENST00000624995 |
| 1:153778173:C:T | rs535256255 | ENST00000271857:ENST00000368661:ENST00000624995 |
| 1:153778174:G:A | rs35102232 | ENST00000271857:ENST00000368661:ENST00000624995 |
| 1:153778174:G:A | rs35102232 | ENST00000271857:ENST00000368661:ENST00000624995 |
| 1:153778176:G:T | | ENST00000271857:ENST00000368661:ENST00000624995 |
| 1:153778176:G:A | | ENST00000271857:ENST00000368661:ENST00000624995 |
| 1:153778177:G:T | | ENST00000271857:ENST00000368661:ENST00000624995 |
| 1:153778177:G:A | rs910853740 | ENST00000271857:ENST00000368661:ENST00000624995 |
| 1:153778177:G:GC | rs144727289; | ENST00000271857:ENST00000368661:ENST00000624995 |
| | rS747973469 | |
| 1:153778179:C:T | rs150299304 | ENST00000271857:ENST00000368661:ENST00000624995 |
| 1:153778180:A:G | rs377578450 | ENST00000271857:ENST00000368661:ENST00000624995 |
| 1:153778182:A:C | | ENST00000271857:ENST00000368661:ENST00000624995 |
| 1:153778183:A:G | | ENST00000271857:ENST00000368661:ENST00000624995 |
| 1:153778183:A:C | | ENST00000271857:ENST00000368661:ENST00000624995 |
| 1:153778183:A:T | | ENST00000271857:ENST00000368661:ENST00000624995 |
| 1:153778184:G:T | rs746442496 | ENST00000271857:ENST00000368661:ENST00000624995 |
| 1:153778185:G:T | | ENST00000271857:ENST00000368661:ENST00000624995 |
| 1:153778185:G:A | rs757804292 | ENST00000271857:ENST00000368661:ENST00000624995 |
| 1:153778186:T:A | | ENST00000271857:ENST00000368661:ENST00000624995 |
| 1:153778188:C:T | rs147176615 | ENST00000271857:ENST00000368661:ENST00000624995 |
| 1:153778188:C:T | rs147176615 | ENST00000271857:ENST00000368661:ENST00000624995 |
| 1:153778189:G:A | rs141932545 | ENST00000271857:ENST00000368661:ENST00000624995 |
| 1:153778189:G:C | | ENST00000271857:ENST00000368661:ENST00000624995 |
| 1:153778189:G:A | rs141932545 | ENST00000271857:ENST00000368661:ENST00000624995 |
| 1:153778192:T:A | | ENST00000271857:ENST00000368661:ENST00000624995 |
| 1:153778194:G:A | | ENST00000271857:ENST00000368661:ENST00000624995 |
| 1:153778195:CAGT | | ENST00000271857:ENST00000368661:ENST00000624995 |
| GG:C | | |
| 1:153778195:C:T | rs747088804 | ENST00000271857:ENST00000368661:ENST00000624995 |
| 1:153778198:T:C | rs768766631 | ENST00000271857:ENST00000368661:ENST00000624995 |
| 1:153778198:T:G | | ENST00000271857:ENST00000368661:ENST00000624995 |
| 1:153778200:G:C | | ENST00000271857:ENST00000368661:ENST00000624995 |
| 1:153778200:G:A | | ENST00000271857:ENST00000368661:ENST00000624995 |
| 1:153778201:GC:G | | ENST00000271857:ENST00000368661:ENST00000624995 |
| 1:153778203:A:T | rs904753440 | ENST00000271857:ENST00000368661:ENST00000624995 |
| 1:153778206:G:T | | ENST00000271857:ENST00000368661:ENST00000624995 |
| 1:153778206:G:A | rs146128753 | ENST00000271857:ENST00000368661:ENST00000624995 |
| 1:153778206:G:A | rs146128753 | ENST00000271857:ENST00000368661:ENST00000624995 |
| 1:153778210:T:C | rs773954711 | ENST00000271857:ENST00000368661:ENST00000624995 |
| 1:153778212:C:T | rs775319912 | ENST00000271857:ENST00000368661:ENST00000624995 |
| 1:153778212:C:A | | ENST00000271857:ENST00000368661:ENST00000624995 |
| 1:153778213:G:T | | ENST00000271857:ENST00000368661:ENST00000624995 |
| 1:153778213:G:A | rs142650546 | ENST00000271857:ENST00000368661:ENST00000624995 |
| 1:153778213:G:A | rs142650546 | ENST00000271857:ENST00000368661:ENST00000624995 |
| 1:153778213:G:C | rs142650546 | ENST00000271857:ENST00000368661:ENST00000624995 |
| 1:153778215:C:T | | ENST00000271857:ENST00000368661:ENST00000624995 |
| 1:153778216:C:T | | ENST00000271857:ENST00000368661:ENST00000624995 |
| 1:153778218:G:A | | ENST00000271857:ENST00000368661:ENST00000624995 |
| 1:153778219:A:T | | ENST00000271857:ENST00000368661:ENST00000624995 |
| 1:153778219:A:G | | ENST00000271857:ENST00000368661:ENST00000624995 |
| 1:153778221:A:G | | ENST00000271857:ENST00000368661:ENST00000624995 |
| 1:153778222:C:A | | ENST00000271857:ENST00000368661:ENST00000624995 |
| 1:153778222:C:T | rs547644986 | ENST00000271857:ENST00000368661:ENST00000624995 |
| 1:153778222:C:G | rs547644986 | ENST00000271857:ENST00000368661:ENST00000624995 |
| 1:153778225:G:A | rs1465960552 | ENST00000271857:ENST00000368661:ENST00000624995 |
| 1:153778226:G:A | | ENST00000271857:ENST00000368661:ENST00000624995 |
| 1:153778227:G:A | rs143578814 | ENST00000271857:ENST00000368661:ENST00000624995 |
| 1:153778227:G:T | rs143578814 | ENST00000271857:ENST00000368661:ENST00000624995 |
| 1:153778228:A:C | | ENST00000271857:ENST00000368661:ENST00000624995 |
| 1:153778229:G:T | | ENST00000271857:ENST00000368661:ENST00000624995 |
| 1:153778230:C:T | rs375994470 | ENST00000271857:ENST00000368661:ENST00000624995 |
| 1:153778230:C:G | | ENST00000271857:ENST00000368661:ENST00000624995 |
| 1:153778231:G:A | rs201939587 | ENST00000271857:ENST00000368661:ENST00000624995 |
| 1:153778234:T:C | rs751831690 | ENST00000271857:ENST00000368661:ENST00000624995 |
| 1:153778236:G:A | | ENST00000271857:ENST00000368661:ENST00000624995 |
| 1:153778237:T:G | rs755217881 | ENST00000271857:ENST00000368661:ENST00000624995 |
| 1:153778239:C:G | rs182081931 | ENST00000271857:ENST00000368661:ENST00000624995 |
| 1:153778239:C:T | rs182081931 | ENST00000271857:ENST00000368661:ENST00000624995 |
| 1:153778240:G:A | rs201437608 | ENST00000271857:ENST00000368661:ENST00000624995 |
| 1:153778240:G:C | | ENST00000271857:ENST00000368661:ENST00000624995 |
| 1:153778240:G:T | rs201437608 | ENST00000271857:ENST00000368661:ENST00000624995 |
| 1:153778242:C:T | rs772039356 | ENST00000271857:ENST00000368661:ENST00000624995 |
| 1:153778243:G:A | rs143908472 | ENST00000271857:ENST00000368661:ENST00000624995 |
| 1:153778243:G:A | rs143908472 | ENST00000271857:ENST00000368661:ENST00000624995 |
| 1:153778245:T:C | rs1292647942 | ENST00000271857:ENST00000368661:ENST00000624995 |
| 1:153778248:GGG | | ENST00000271857:ENST00000368661:ENST00000624995 |
| CCCCTGCA:G | | |
| 1:153778248:G:A | rs372623621 | ENST00000271857:ENST00000368661:ENST00000624995 |
| 1:153778248:G:C | | ENST00000271857:ENST00000368661:ENST00000624995 |
| 1:153778248:G:T | | ENST00000271857:ENST00000368661:ENST00000624995 |
| 1:153778249:GGC: | | ENST00000271857:ENST00000368661:ENST00000624995 |
| G | | |
| 1:153778250:G:GC | rs770078882 | ENST00000271857:ENST00000368661:ENST00000624995 |
| 1:153778251:C:T | rs1303528906 | ENST00000271857:ENST00000368661:ENST00000624995 |
| 1:153778252:C:T | | ENST00000271857:ENST00000368661:ENST00000624995 |
| 1:153778254:CT:C | rs773333339 | ENST00000271857:ENST00000368661:ENST00000624995 |
| 1:153778255:T:G | | ENST00000271857:ENST00000368661:ENST00000624995 |
| 1:153778255:T:A | rs1311383969 | ENST00000271857:ENST00000368661:ENST00000624995 |
| 1:153778258:A:G | | ENST00000271857:ENST00000368661:ENST00000624995 |
| 1:153778266:G:A | | ENST00000271857:ENST00000368661:ENST00000624995 |
| 1:153778267:A:G | rs766668912 | ENST00000271857:ENST00000368661:ENST00000624995 |
| 1:153778270:C:T | | ENST00000271857:ENST00000368661:ENST00000624995 |
| 1:153778270:CAT:C | rs763035632 | ENST00000271857:ENST00000368661:ENST00000624995 |
| 1:153778272:T:C | | ENST00000271857:ENST00000368661:ENST00000624995 |
| 1:153778273:A:G | | ENST00000271857:ENST00000368661:ENST00000624995 |
| 1:153778274:T:G | rs139337136 | ENST00000271857:ENST00000368661:ENST00000624995 |
| 1:153778275:G:A | rs979482736 | ENST00000271857:ENST00000368661:ENST00000624995 |
| 1:153778276:G:A | | ENST00000271857:ENST00000368661:ENST00000624995 |
| 1:153778278:C:A | | ENST00000271857:ENST00000368661:ENST00000624995 |
| 1:153778282:C:T | rs1448912602 | ENST00000271857:ENST00000368661:ENST00000624995 |
| 1:153778282:CAG: | rs1491238717 | ENST00000271857:ENST00000368661:ENST00000624995 |
| C | | |
| 1:153778287:G:A | rs1185358227 | ENST00000271857:ENST00000368661:ENST00000624995 |
| 1:153778288:G:A | rs1422259463 | ENST00000271857:ENST00000368661:ENST00000624995 |
| 1:153778291:A:G | | ENST00000271857:ENST00000368661:ENST00000624995 |
| 1:153778293:G:A | rs139458875 | ENST00000271857:ENST00000368661:ENST00000624995 |
| 1:153778293:G:T | | ENST00000271857:ENST00000368661:ENST00000624995 |
| 1:153778293:G:C | rs139458875 | ENST00000271857:ENST00000368661:ENST00000624995 |
| 1:153778293:G:A | rs139458875 | ENST00000271857:ENST00000368661:ENST00000624995 |
| 1:153778294:T:C | | ENST00000271857:ENST00000368661:ENST00000624995 |
| 1:153778297:C:T | | ENST00000271857:ENST00000368661:ENST00000624995 |
| 1:153778300:C:G | | ENST00000271857:ENST00000368661:ENST00000624995 |
| 1:153778300:C:T | | ENST00000271857:ENST00000368661:ENST00000624995 |
| 1:153778302:A:G | rs758614499 | ENST00000271857:ENST00000368661:ENST00000624995 |
| 1:153778303:T:C | | ENST00000271857:ENST00000368661:ENST00000624995 |
| 1:153778305:A:G | | ENST00000271857:ENST00000368661:ENST00000624995 |
| 1:153778306:A:G | | ENST00000271857:ENST00000368661:ENST00000624995 |
| 1:153778306:A:T | rs746822570 | ENST00000271857:ENST00000368661:ENST00000624995 |
| 1:153778307:C:G | rs141993722 | ENST00000271857:ENST00000368661:ENST00000624995 |
| 1:153778308:T:C | rs1285601842 | ENST00000271857:ENST00000368661:ENST00000624995 |
| 1:153778310:C:G | | ENST00000271857:ENST00000368661:ENST00000624995 |
| 1:153778311:A:G | rs747605041 | ENST00000271857:ENST00000368661:ENST00000624995 |
| 1:153778312:C:G | rs141186570 | ENST00000271857:ENST00000368661:ENST00000624995 |
| 1:153778312:C:T | rs141186570 | ENST00000271857:ENST00000368661:ENST00000624995 |
| 1:153778314:G:A | rs376964990 | ENST00000271857:ENST00000368661:ENST00000624995 |
| 1:153778320:C:T | rs772719483 | ENST00000271857:ENST00000368661:ENST00000624995 |
| 1:153778321:G:C | | ENST00000271857:ENST00000368661:ENST00000624995 |
| 1:153778321:G:A | rs374899034 | ENST00000271857:ENST00000368661:ENST00000624995 |
| 1:153778323:G:T | | ENST00000271857:ENST00000368661:ENST00000624995 |
| 1:153778323:G:C | | ENST00000271857:ENST00000368661:ENST00000624995 |
| 1:153778324:G:A | | ENST00000271857:ENST00000368661:ENST00000624995 |
| 1:153778324:G:T | | ENST00000271857:ENST00000368661:ENST00000624995 |
| 1:153778324:G:C | | ENST00000271857:ENST00000368661:ENST00000624995 |
| 1:153778326:G:A | rs150774960 | ENST00000271857:ENST00000368661:ENST00000624995 |
| 1:153778326:G:A | rs150774960 | ENST00000271857:ENST00000368661:ENST00000624995 |
| 1:153778327:CT:C | | ENST00000271857:ENST00000368661:ENST00000624995 |
| 1:153778327:C:A | | ENST00000271857:ENST00000368661:ENST00000624995 |
| 1:153778333:G:A | rs759989805 | ENST00000271857:ENST00000368661:ENST00000624995 |
| 1:153778333:G:T | rs759989805 | ENST00000271857:ENST00000368661:ENST00000624995 |
| 1:153778335:C:T | rs140637267 | ENST00000271857:ENST00000368661:ENST00000624995 |
| 1:153778336:G:GT | | ENST00000271857:ENST00000368661:ENST00000624995 |
| GCT | | |
| 1:153778336:G:A | rs199869514 | ENST00000271857:ENST00000368661:ENST00000624995 |
| 1:153778336:G:C | | ENST00000271857:ENST00000368661:ENST00000624995 |
| 1:153778336:G:A | rs199869514 | ENST00000271857:ENST00000368661:ENST00000624995 |
| 1:153778336:G:T | | ENST00000271857:ENST00000368661:ENST00000624995 |
| 1:153778339:C:G | | ENST00000271857:ENST00000368661:ENST00000624995 |
| 1:153778339:C:T | | ENST00000271857:ENST00000368661:ENST00000624995 |
| 1:153778341:T:C | | ENST00000271857:ENST00000368661:ENST00000624995 |
| 1:153778341:T:A | rs1181764460 | ENST00000271857:ENST00000368661:ENST00000624995 |
| 1:153778344:T:G | | ENST00000271857:ENST00000368661:ENST00000624995 |
| 1:153778345:G:A | rs756306439 | ENST00000271857:ENST00000368661:ENST00000624995 |
| 1:153778348:T:C | rs1165268294 | ENST00000271857:ENST00000368661:ENST00000624995 |
| 1:153778350:T:C | rs1174624690 | ENST00000271857:ENST00000368661:ENST00000624995 |
| 1:153778353:A:G | | ENST00000271857:ENST00000368661:ENST00000624995 |
| 1:153778354:A:C | | ENST00000271857:ENST00000368661:ENST00000624995 |
| 1:153778355:G:T | | ENST00000271857:ENST00000368661:ENST00000624995 |
| 1:153778356:G:A | rs765174664 | ENST00000271857:ENST00000368661:ENST00000624995 |
| 1:153778356:G:T | | ENST00000271857:ENST00000368661:ENST00000624995 |
| 1:153778357:T:G | | ENST00000271857:ENST00000368661:ENST00000624995 |
| 1:153778461:A:C | | ENST00000271857:ENST00000368661:ENST00000624995 |
| 1:153778462:G:GC | | ENST00000271857:ENST00000368661:ENST00000624995 |
| A | | |
| 1:153778463:C:T | rs1183626517 | ENST00000271857:ENST00000368661:ENST00000624995 |
| 1:153778464:A:G | rs755739082 | ENST00000271857:ENST00000368661:ENST00000624995 |
| 1:153778466:A:G | | ENST00000271857:ENST00000368661:ENST00000624995 |
| 1:153778469:T:A | rs777383360 | ENST00000271857:ENST00000368661:ENST00000624995 |
| 1:153778471:C:A | | ENST00000271857:ENST00000368661:ENST00000624995 |
| 1:153778472:C:T | rs150289679 | ENST00000271857:ENST00000368661:ENST00000624995 |
| 1:153778473:C:T | rs778411361 | ENST00000271857:ENST00000368661:ENST00000624995 |
| 1:153778475:T:C | rs1394148660 | ENST00000271857:ENST00000368661:ENST00000624995 |
| 1:153778477:C:G | | ENST00000271857:ENST00000368661:ENST00000624995 |
| 1:153778479:C:G | | ENST00000271857:ENST00000368661:ENST00000624995 |
| 1:153778482:T:C | rs200502524 | ENST00000271857:ENST00000368661:ENST00000624995 |
| 1:153778483:G:C | rs1336584051 | ENST00000271857:ENST00000368661:ENST00000624995 |
| 1:153778483:G:T | | ENST00000271857:ENST00000368661:ENST00000624995 |
| 1:153778484:A:G | rs775876675 | ENST00000271857:ENST00000368661:ENST00000624995 |
| 1:153778485:T:C | rs760994157 | ENST00000271857:ENST00000368661:ENST00000624995 |
| 1:153778486:T:G | | ENST00000271857:ENST00000368661:ENST00000624995 |
| 1:153778487:C:T | rs143621188 | ENST00000271857:ENST00000368661:ENST00000624995 |
| 1:153778487:C:G | rs143621188 | ENST00000271857:ENST00000368661:ENST00000624995 |
| 1:153778488:G:A | rs189944468 | ENST00000271857:ENST00000368661:ENST00000624995 |
| 1:153778488:G:C | rs189944468 | ENST00000271857:ENST00000368661:ENST00000624995 |
| 1:153778488:G:T | | ENST00000271857:ENST00000368661:ENST00000624995 |
| 1:153778488:G:C | rs189944468 | ENST00000271857:ENST00000368661:ENST00000624995 |
| 1:153778489:CTA:C | | ENST00000271857:ENST00000368661:ENST00000624995 |
| 1:153778491:A:G | rs1185745382 | ENST00000271857:ENST00000368661:ENST00000624995 |
| 1:153778492:T:G | rs750471359 | ENST00000271857:ENST00000368661:ENST00000624995 |
| 1:153778493:G:A | rs1216598733 | ENST00000271857:ENST00000368661:ENST00000624995 |
| 1:153778494:A:T | | ENST00000271857:ENST00000368661:ENST00000624995 |
| 1:153778497:T:G | rs368706417 | ENST00000271857:ENST00000368661:ENST00000624995 |
| 1:153778499:A:G | rs767373557 | ENST00000271857:ENST00000368661:ENST00000624995 |
| 1:153778499:A:C | | ENST00000271857:ENST00000368661:ENST00000624995 |
| 1:153778500:C:A | | ENST00000271857:ENST00000368661:ENST00000624995 |
| 1:153778503:C:G | | ENST00000271857:ENST00000368661:ENST00000624995 |
| 1:153778503:C:T | rs1319494307 | ENST00000271857:ENST00000368661:ENST00000624995 |
| 1:153778505:G:T | | ENST00000271857:ENST00000368661:ENST00000624995 |
| 1:153778505:G:A | rs1274076772 | ENST00000271857:ENST00000368661:ENST00000624995 |
| 1:153778508:G:A | | ENST00000271857:ENST00000368661:ENST00000624995 |
| 1:153778508:G:C | | ENST00000271857:ENST00000368661:ENST00000624995 |
| 1:153778509:A:AA | | ENST00000271857:ENST00000368661:ENST00000624995 |
| AACCCGTGGAACA | | |
| GCAGAGGGCTGGC | | |
| AGGAGAGG | | |
| 1:153778509:A:AA | | ENST00000271857:ENST00000368661:ENST00000624995 |
| AACCCGTGGAACA | | |
| GCAGAGGGCTGGC | | |
| AGGAGAG | | |
| 1:153778511:C:A | rs752510425 | ENST00000271857:ENST00000368661:ENST00000624995 |
| 1:153778512:C:T | rs1254375263 | ENST00000271857:ENST00000368661:ENST00000624995 |
| 1:153778514:A:G | | ENST00000271857:ENST00000368661:ENST00000624995 |
| 1:153778517:C:T | rs137908170 | ENST00000271857:ENST00000368661:ENST00000624995 |
| 1:153778517:C:T | rs137908170 | ENST00000271857:ENST00000368661:ENST00000624995 |
| 1:153778518:G:A | rs142414300 | ENST00000271857:ENST00000368661:ENST00000624995 |
| 1:153778518:G:A | rs142414300 | ENST00000271857:ENST00000368661:ENST00000624995 |
| 1:153778521:A:G | | ENST00000271857:ENST00000368661:ENST00000624995 |
| 1:153778521:A:C | rs1371279423 | ENST00000271857:ENST00000368661:ENST00000624995 |
| 1:153778521:A:T | rs1371279423 | ENST00000271857:ENST00000368661:ENST00000624995 |
| 1:153778522:C:A | rs1463400017 | ENST00000271857:ENST00000368661:ENST00000624995 |
| 1:153778523:C:A | | ENST00000271857:ENST00000368661:ENST00000624995 |
| 1:153778523:C:T | rs1400258336 | ENST00000271857:ENST00000368661:ENST00000624995 |
| 1:153778524:C:T | | ENST00000271857:ENST00000368661:ENST00000624995 |
| 1:153778524:C:A | | ENST00000271857:ENST00000368661:ENST00000624995 |
| 1:153778526:CA:C | rs753001506 | ENST00000271857:ENST00000368661:ENST00000624995 |
| 1:153778527:A:AG | | ENST00000271857:ENST00000368661:ENST00000624995 |
| 1:153778529:G:A | | ENST00000271857:ENST00000368661:ENST00000624995 |
| 1:153778530:G:A | rs753433487 | ENST00000271857:ENST00000368661:ENST00000624995 |
| 1:153778532:C:T | rs745383068 | ENST00000271857:ENST00000368661:ENST00000624995 |
| 1:153778533:A:G | | ENST00000271857:ENST00000368661:ENST00000624995 |
| 1:153778534:C:A | | ENST00000271857:ENST00000368661:ENST00000624995 |
| 1:153778535:T:C | rs950326837 | ENST00000271857:ENST00000368661:ENST00000624995 |
| 1:153778536:G:A | rs756909719 | ENST00000271857:ENST00000368661:ENST00000624995 |
| 1:153778536:G:C | rs756909719 | ENST00000271857:ENST00000368661:ENST00000624995 |
| 1:153778538:A:G | | ENST00000271857:ENST00000368661:ENST00000624995 |
| 1:153778540:G:A | rs1325208994 | ENST00000271857:ENST00000368661:ENST00000624995 |
| 1:153778541:G:A | | ENST00000271857:ENST00000368661:ENST00000624995 |
| 1:153778541:G:C | rs778449064 | ENST00000271857:ENST00000368661:ENST00000624995 |
| 1:153778542:C:G | | ENST00000271857:ENST00000368661:ENST00000624995 |
| 1:153778542:C:T | rs1045854293 | ENST00000271857:ENST00000368661:ENST00000624995 |
| 1:153778544:A:G | | ENST00000271857:ENST00000368661:ENST00000624995 |
| 1:153778548:C:T | rs772610632 | ENST00000271857:ENST00000368661:ENST00000624995 |
| 1:153778550:C:A | | ENST00000271857:ENST00000368661:ENST00000624995 |
| 1:153778550:C:G | | ENST00000271857:ENST00000368661:ENST00000624995 |
| 1:153778553:G:C | rs1204039373 | ENST00000271857:ENST00000368661:ENST00000624995 |
| 1:153778553:G:A | | ENST00000271857:ENST00000368661:ENST00000624995 |
| 1:153778553:G:T | | ENST00000271857:ENST00000368661:ENST00000624995 |
| 1:153778685:A:C | | ENST00000271857:ENST00000368661:ENST00000624995 |
| 1:153778685:A:G | rs1190443217 | ENST00000271857:ENST00000368661:ENST00000624995 |
| 1:153778686:G:C | | ENST00000271857:ENST00000368661:ENST00000624995 |
| 1:153778687:G:GT | | ENST00000271857:ENST00000368661:ENST00000624995 |
| 1:153778689:G:A | rs781653260 | ENST00000271857:ENST00000368661:ENST00000624995 |
| 1:153778692:C:T | | ENST00000271857:ENST00000368661:ENST00000624995 |
| 1:153778693:C:G | | ENST00000271857:ENST00000368661:ENST00000624995 |
| 1:153778693:CA:C | rs750093955 | ENST00000271857:ENST00000368661:ENST00000624995 |
| 1:153778695:G:A | | ENST00000271857:ENST00000368661:ENST00000624995 |
| 1:153778695:G:C | | ENST00000271857:ENST00000368661:ENST00000624995 |
| 1:153778696:G:A | rs1352432234 | ENST00000271857:ENST00000368661:ENST00000624995 |
| 1:153778696:G:T | | ENST00000271857:ENST00000368661:ENST00000624995 |
| 1:153778699:T:C | | ENST00000271857:ENST00000368661:ENST00000624995 |
| 1:153778704:G:A | | ENST00000271857:ENST00000368661:ENST00000624995 |
| 1:153778705:T:C | | ENST00000271857:ENST00000368661:ENST00000624995 |
| 1:153778707:GC:G | | ENST00000271857:ENST00000368661:ENST00000624995 |
| 1:153778708:C:A | rs773343272 | ENST00000271857:ENST00000368661:ENST00000624995 |
| 1:153778710:C:T | rs200097042 | ENST00000271857:ENST00000368661:ENST00000624995 |
| 1:153778710:C:G | | ENST00000271857:ENST00000368661:ENST00000624995 |
| 1:153778711:C:T | rs547501540 | ENST00000271857:ENST00000368661:ENST00000624995 |
| 1:153778713:G:GT | rs757896214 | ENST00000271857:ENST00000368661:ENST00000624995 |
| 1:153778717:G:T | | ENST00000271857:ENST00000368661:ENST00000624995 |
| 1:153778717:G:A | | ENST00000271857:ENST00000368661:ENST00000624995 |
| 1:153778722:C:T | rs1369925156 | ENST00000271857:ENST00000368661:ENST00000624995 |
| 1:153778723:A:G | | ENST00000271857:ENST00000368661:ENST00000624995 |
| 1:153778725:T:C | | ENST00000271857:ENST00000368661:ENST00000624995 |
| 1:153778726:C:A | | ENST00000271857:ENST00000368661:ENST00000624995 |
| 1:153778728:C:T | rs1248699174 | ENST00000271857:ENST00000368661:ENST00000624995 |
| 1:153778728:C:G | | ENST00000271857:ENST00000368661:ENST00000624995 |
| 1:153778729:C:T | rs1399471528 | ENST00000271857:ENST00000368661:ENST00000624995 |
| 1:153778735:TG:T | rs1159358491 | ENST00000271857:ENST00000368661:ENST00000624995 |
| 1:153778737:G:A | rs1433455597 | ENST00000271857:ENST00000368661:ENST00000624995 |
| 1:153778738:G:A | | ENST00000271857:ENST00000368661:ENST00000624995 |
| 1:153778738:G:T | rs1176043639 | ENST00000271857:ENST00000368661:ENST00000624995 |
| 1:153778740:T:C | rs141064582 | ENST00000271857:ENST00000368661:ENST00000624995 |
| 1:153778741:ATGC | | ENST00000271857:ENST00000368661:ENST00000624995 |
| TGGCGGGCCAGAG | | |
| C:A | | |
| 1:153778742T:A | | ENST00000271857:ENST00000368661:ENST00000624995 |
| 1:153778743:G:A | | ENST00000271857:ENST00000368661:ENST00000624995 |
| 1:153778744:C:T | | ENST00000271857:ENST00000368661:ENST00000624995 |
| 1:153778744:C:A | | ENST00000271857:ENST00000368661:ENST00000624995 |
| 1:153778746:G:A | | ENST00000271857:ENST00000368661:ENST00000624995 |
| 1:153778746:G:C | rs1001146776 | ENST00000271857:ENST00000368661:ENST00000624995 |
| 1:153778749:G:A | rs369621949 | ENST00000271857:ENST00000368661:ENST00000624995 |
| 1:153778749:G:C | | ENST00000271857:ENST00000368661:ENST00000624995 |
| 1:153778750:G:A | rs750100094 | ENST00000271857:ENST00000368661:ENST00000624995 |
| 1:153778753:C:T | rs1270591468 | ENST00000271857:ENST00000368661:ENST00000624995 |
| 1:153778756:A:C | | ENST00000271857:ENST00000368661:ENST00000624995 |
| 1:153778757:G:T | | ENST00000271857:ENST00000368661:ENST00000624995 |
| 1:153778758:CTGG | | ENST00000271857:ENST00000368661:ENST00000624995 |
| CCCAGGGG:C | | |
| 1:153778758:C:G | | ENST00000271857:ENST00000368661:ENST00000624995 |
| 1:153778761:G:C | | ENST00000271857:ENST00000368661:ENST00000624995 |
| 1:153778762:C:T | rs765738547 | ENST00000271857:ENST00000368661:ENST00000624995 |
| 1:153778764:C:G | | ENST00000271857:ENST00000368661:ENST00000624995 |
| 1:153778764:C:T | rs751144146 | ENST00000271857:ENST00000368661:ENST00000624995 |
| 1:153778765:A:G | | ENST00000271857:ENST00000368661:ENST00000624995 |
| 1:153778771:A:C | | ENST00000271857:ENST00000368661:ENST00000624995 |
| 1:153778771:A:G | rs147963978 | ENST00000271857:ENST00000368661:ENST00000624995 |
| 1:153778771:A:G | rs147963978 | ENST00000271857:ENST00000368661:ENST00000624995 |
| 1:153778772:G:T | rs971593239 | ENST00000271857:ENST00000368661:ENST00000624995 |
| 1:153778776:C:G | rs377442396 | ENST00000271857:ENST00000368661:ENST00000624995 |
| 1:153778777:T:C | | ENST00000271857:ENST00000368661:ENST00000624995 |
| 1:153778780:A:G | | ENST00000271857:ENST00000368661:ENST00000624995 |
| 1:153778780:A:C | rs756498765 | ENST00000271857:ENST00000368661:ENST00000624995 |
| 1:153778782:G:A | | ENST00000271857:ENST00000368661:ENST00000624995 |
| 1:153778782:G:C | | ENST00000271857:ENST00000368661:ENST00000624995 |
| 1:153778783:A:G | | ENST00000271857:ENST00000368661:ENST00000624995 |
| 1:153778784:T:A | rs778216359 | ENST00000271857:ENST00000368661:ENST00000624995 |
| 1:153778785:G:A | | ENST00000271857:ENST00000368661:ENST00000624995 |
| 1:153778789:T:C | | ENST00000271857:ENST00000368661:ENST00000624995 |
| 1:153778791:C:T | rs138614386 | ENST00000271857:ENST00000368661:ENST00000624995 |
| 1:153778791:C:T | rs138614386 | ENST00000271857:ENST00000368661:ENST00000624995 |
| 1:153778792:G:A | rs771218990 | ENST00000271857:ENST00000368661:ENST00000624995 |
| 1:153778792:G:C | | ENST00000271857:ENST00000368661:ENST00000624995 |
| 1:153778795:C:T | | ENST00000271857:ENST00000368661:ENST00000624995 |
| 1:153778796:TG:T | | ENST00000271857:ENST00000368661:ENST00000624995 |
| 1:153778798:G:A | | ENST00000271857:ENST00000368661:ENST00000624995 |
| 1:153778800:G:T | | ENST00000271857:ENST00000368661:ENST00000624995 |
| 1:153778800:G:A | | ENST00000271857:ENST00000368661:ENST00000624995 |
| 1:153778801:A:T | rs1252493937 | ENST00000271857:ENST00000368661:ENST00000624995 |
| 1:153778801:A:G | | ENST00000271857:ENST00000368661:ENST00000624995 |
| 1:153778802:T:G | | ENST00000271857:ENST00000368661:ENST00000624995 |
| 1:153778803:G:C | | ENST00000271857:ENST00000368661:ENST00000624995 |
| 1:153778803:G:A | | ENST00000271857:ENST00000368661:ENST00000624995 |
| 1:153778804:T:C | | ENST00000271857:ENST00000368661:ENST00000624995 |
| 1:153778807:T:C | | ENST00000271857:ENST00000368661:ENST00000624995 |
| 1:153778808:C:G | | ENST00000271857:ENST00000368661:ENST00000624995 |
| 1:153778810:T:C | | ENST00000271857:ENST00000368661:ENST00000624995 |
| 1:153778812:A:C | rs768550208 | ENST00000271857:ENST00000368661:ENST00000624995 |
| 1:153778815:A:T | | ENST00000271857:ENST00000368661:ENST00000624995 |
| 1:153778816:C:G | | ENST00000271857:ENST00000368661:ENST00000624995 |
| 1:153778816:C:T | | ENST00000271857:ENST00000368661:ENST00000624995 |
| 1:153778816:C:A | | ENST00000271857:ENST00000368661:ENST00000624995 |
| 1:153778818:G:T | | ENST00000271857:ENST00000368661:ENST00000624995 |
| 1:153778819:G:C | rs761581765 | ENST00000271857:ENST00000368661:ENST00000624995 |
| 1:153778819:G:A | rs761581765 | ENST00000271857:ENST00000368661:ENST00000624995 |
| 1:153778822:A:C | rs1218297742 | ENST00000271857:ENST00000368661:ENST00000624995 |
| 1:153778824:C:G | | ENST00000271857:ENST00000368661:ENST00000624995 |
| 1:153778825:T:C | | ENST00000271857:ENST00000368661:ENST00000624995 |
| 1:153778825:T:G | rs773063098 | ENST00000271857:ENST00000368661:ENST00000624995 |
| 1:153778833:T:C | | ENST00000271857:ENST00000368661:ENST00000624995 |
| 1:153778835:C:G | rs145965895 | ENST00000271857:ENST00000368661:ENST00000624995 |
| 1:153778836:G:A | rs141752584 | ENST00000271857:ENST00000368661:ENST00000624995 |
| 1:153778836:G:A | rs141752584 | ENST00000271857:ENST00000368661:ENST00000624995 |
| 1:153778837:A:G | rs768130983 | ENST00000271857:ENST00000368661:ENST00000624995 |
| 1:153778838:T:A | rs536824983 | ENST00000271857:ENST00000368661:ENST00000624995 |
| 1:153778841:C:G | | ENST00000271857:ENST00000368661:ENST00000624995 |
| 1:153778842:C:T | | ENST00000271857:ENST00000368661:ENST00000624995 |
| 1:153778843:A:G | | ENST00000271857:ENST00000368661:ENST00000624995 |
| 1:153778845:G:A | rs756653941 | ENST00000271857:ENST00000368661:ENST00000624995 |
| 1:153778846:G:T | rs555781194 | ENST00000271857:ENST00000368661:ENST00000624995 |
| 1:153778846:G:A | rs555781194 | ENST00000271857:ENST00000368661:ENST00000624995 |
| 1:153778851:C:T | | ENST00000271857:ENST00000368661:ENST00000624995 |
| 1:153778854:C:T | rs140437865 | ENST00000271857:ENST00000368661:ENST00000624995 |
| 1:153778854:C:G | | ENST00000271857:ENST00000368661:ENST00000624995 |
| 1:153778855:G:A | rs138771667 | ENST00000271857:ENST00000368661:ENST00000624995 |
| 1:153778855:G:T | | ENST00000271857:ENST00000368661:ENST00000624995 |
| 1:153778855:G:A | rs138771667 | ENST00000271857:ENST00000368661:ENST00000624995 |
| 1:153778858:T:A | | ENST00000271857:ENST00000368661:ENST00000624995 |
| 1:153778860:C:T | rs772199009 | ENST00000271857:ENST00000368661:ENST00000624995 |
| 1:153778861:A:G | rs1478343134 | ENST00000271857:ENST00000368661:ENST00000624995 |
| 1:153778863:G:T | | ENST00000271857:ENST00000368661:ENST00000624995 |
| 1:153778863:G:A | rs780120241 | ENST00000271857:ENST00000368661:ENST00000624995 |
| 1:153778864:A:G | rs146302140 | ENST00000271857:ENST00000368661:ENST00000624995 |
| 1:153778864:A:T | rs146302140 | ENST00000271857:ENST00000368661:ENST00000624995 |
| 1:153778866:C:A | | ENST00000271857:ENST00000368661:ENST00000624995 |
| 1:153778866:C:T | rs374567006 | ENST00000271857:ENST00000368661:ENST00000624995 |
| 1:153778867:G:A | rs202116475 | ENST00000271857:ENST00000368661:ENST00000624995 |
| 1:153778870:C:A | rs770673905 | ENST00000271857:ENST00000368661:ENST00000624995 |
| 1:153778875:G:A | | ENST00000271857:ENST00000368661:ENST00000624995 |
| 1:153778876:A:G | | ENST00000271857:ENST00000368661:ENST00000624995 |
| 1:153778878:A:T | | ENST00000271857:ENST00000368661:ENST00000624995 |
| 1:153778878:A:C | | ENST00000271857:ENST00000368661:ENST00000624995 |
| 1:153778879:C:T | | ENST00000271857:ENST00000368661:ENST00000624995 |
| 1:153778880:C:CT | | ENST00000271857:ENST00000368661:ENST00000624995 |
| 1:153778881:T:G | | ENST00000271857:ENST00000368661:ENST00000624995 |
| 1:153778881:T:C | | ENST00000271857:ENST00000368661:ENST00000624995 |
| 1:153778883:C:G | rs762166253 | ENST00000271857:ENST00000368661:ENST00000624995 |
| 1:153778884:A:G | rs1000687067 | ENST00000271857:ENST00000368661:ENST00000624995 |
| 1:153778886:G:A | rs377369659 | ENST00000271857:ENST00000368661:ENST00000624995 |
| 1:153778886:G:T | | ENST00000271857:ENST00000368661:ENST00000624995 |
| 1:153779113:G:A | | ENST00000271857:ENST00000368661:ENST00000624995 |
| 1:153779113:G:T | | ENST00000271857:ENST00000368661:ENST00000624995 |
| 1:153779114:G:C | rs930038793 | ENST00000271857:ENST00000368661:ENST00000624995 |
| 1:153779116:G:A | rs1380326354 | ENST00000271857:ENST00000368661:ENST00000624995 |
| 1:153779118:A:C | rs1173730214 | ENST00000271857:ENST00000368661:ENST00000624995 |
| 1:153779119:A:AG | | ENST00000271857:ENST00000368661:ENST00000624995 |
| 1:153779120:G:C | | ENST00000271857:ENST00000368661:ENST00000624995 |
| 1:153779120:G:T | | ENST00000271857:ENST00000368661:ENST00000624995 |
| 1:153779121:G:T | rs200472466 | ENST00000271857:ENST00000368661:ENST00000624995 |
| 1:153779121:G:C | rs200472466 | ENST00000271857:ENST00000368661:ENST00000624995 |
| 1:153779122:G:T | | ENST00000271857:ENST00000368661:ENST00000624995 |
| 1:153779122:G:A | rs774295555 | ENST00000271857:ENST00000368661:ENST00000624995 |
| 1:153779122:G:C | | ENST00000271857:ENST00000368661:ENST00000624995 |
| 1:153779124:G:A | rs1224726568 | ENST00000271857:ENST00000368661:ENST00000624995 |
| 1:153779129:T:A | | ENST00000271857:ENST00000368661:ENST00000624995 |
| 1:153779130:G:A | | ENST00000271857:ENST00000368661:ENST00000624995 |
| 1:153779134:C:T | | ENST00000271857:ENST00000368661:ENST00000624995 |
| 1:153779136:A:G | | ENST00000271857:ENST00000368661:ENST00000624995 |
| 1:153779137:C:A | | ENST00000271857:ENST00000368661:ENST00000624995 |
| 1:153779137:C:CA | | ENST00000271857:ENST00000368661:ENST00000624995 |
| 1:153779137:C:G | | ENST00000271857:ENST00000368661:ENST00000624995 |
| 1:153779139:A:G | rs771875829 | ENST00000271857:ENST00000368661:ENST00000624995 |
| 1:153779140:C:T | | ENST00000271857:ENST00000368661:ENST00000624995 |
| 1:153779140:C:G | | ENST00000271857:ENST00000368661:ENST00000624995 |
| 1:153779142:G:A | rs547168142 | ENST00000271857:ENST00000368661:ENST00000624995 |
| 1:153779142:G:T | | ENST00000271857:ENST00000368661:ENST00000624995 |
| 1:153779145:G:A | rs766408586 | ENST00000271857:ENST00000368661:ENST00000624995 |
| 1:153779146:T:C | | ENST00000271857:ENST00000368661:ENST00000624995 |
| 1:153779148:G:A | | ENST00000271857:ENST00000368661:ENST00000624995 |
| 1:153779149:C:T | rs1377714473 | ENST00000271857:ENST00000368661:ENST00000624995 |
| 1:153779154:G:T | rs1278808046 | ENST00000271857:ENST00000368661:ENST00000624995 |
| 1:153779155:T:A | | ENST00000271857:ENST00000368661:ENST00000624995 |
| 1:153779158:T:C | | ENST00000271857:ENST00000368661:ENST00000624995 |
| 1:153779158:TC:T | rs773458443 | ENST00000271857:ENST00000368661:ENST00000624995 |
| 1:153779159:C:A | rs144843954 | ENST00000271857:ENST00000368661:ENST00000624995 |
| 1:153779160:G:C | | ENST00000271857:ENST00000368661:ENST00000624995 |
| 1:153779160:G:A | rs750817089 | ENST00000271857:ENST00000368661:ENST00000624995 |
| 1:153779161:A:G | rs1294576727 | ENST00000271857:ENST00000368661:ENST00000624995 |
| 1:153779162:G:T | | ENST00000271857:ENST00000368661:ENST00000624995 |
| 1:153779163:G:A | | ENST00000271857:ENST00000368661:ENST00000624995 |
| 1:153779164:C:T | rs758565684 | ENST00000271857:ENST00000368661:ENST00000624995 |
| 1:153779170:A:G | rs141869816 | ENST00000271857:ENST00000368661:ENST00000624995 |
| 1:153779178:C:T | | ENST00000271857:ENST00000368661:ENST00000624995 |
| 1:153779179:A:G | | ENST00000271857:ENST00000368661:ENST00000624995 |
| 1:153779184:G:A | rs749188333 | ENST00000271857:ENST00000368661:ENST00000624995 |
| 1:153779185:T:G | | ENST00000271857:ENST00000368661:ENST00000624995 |
| 1:153779185:T:C | rs757296206 | ENST00000271857:ENST00000368661:ENST00000624995 |
| 1:153779189:C:A | rs752655899 | ENST00000271857:ENST00000368661:ENST00000624995 |
| 1:153779190:G:A | rs775281896 | ENST00000271857:ENST00000368661:ENST00000624995 |
| 1:153779191:T:C | rs140137601 | ENST00000271857:ENST00000368661:ENST00000624995 |
| 1:153779194:A:G | rs1376126558 | ENST00000271857:ENST00000368661:ENST00000624995 |
| 1:153779195:T:G | | ENST00000271857:ENST00000368661:ENST00000624995 |
| 1:153779196:G:T | | ENST00000271857:ENST00000368661:ENST00000624995 |
| 1:153779196:G:A | rs768156076 | ENST00000271857:ENST00000368661:ENST00000624995 |
| 1:153779197:G:C | rs1440270337 | ENST00000271857:ENST00000368661:ENST00000624995 |
| 1:153779197:G:T | | ENST00000271857:ENST00000368661:ENST00000624995 |
| 1:153779199:G:A | | ENST00000271857:ENST00000368661:ENST00000624995 |
| 1:153779200:T:A | | ENST00000271857:ENST00000368661:ENST00000624995 |
| 1:153779203:C:G | | ENST00000271857:ENST00000368661:ENST00000624995 |
| 1:153779205:G:C | rs373575068 | ENST00000271857:ENST00000368661:ENST00000624995 |
| 1:153779205:G:T | | ENST00000271857:ENST00000368661:ENST00000624995 |
| 1:153779206:T:C | rs1270019035 | ENST00000271857:ENST00000368661:ENST00000624995 |
| 1:153779208:C:T | | ENST00000271857:ENST00000368661:ENST00000624995 |
| 1:153779209:C:T | | ENST00000271857:ENST00000368661:ENST00000624995 |
| 1:153779212:G:A | rs766501697 | ENST00000271857:ENST00000368661:ENST00000624995 |
| 1:153779213:T:C | rs751863190 | ENST00000271857:ENST00000368661:ENST00000624995 |
| 1:153779341:A:AG | rs1295602822 | ENST00000271857:ENST00000368661:ENST00000624995 |
| GGCATGAAGGCAG | | |
| GGC | | |
| 1:153779342:G:A | | ENST00000271857:ENST00000368661:ENST00000624995 |
| 1:153779343:G:T | | ENST00000271857:ENST00000368661:ENST00000624995 |
| 1:153779343:G:C | | ENST00000271857:ENST00000368661:ENST00000624995 |
| 1:153779343:G:A | rs750328049 | ENST00000271857:ENST00000368661:ENST00000624995 |
| 1:153779345:C:T | | ENST00000271857:ENST00000368661:ENST00000624995 |
| 1:153779346:A:G | rs779860149 | ENST00000271857:ENST00000368661:ENST00000624995 |
| 1:153779347:T:G | rs144962694 | ENST00000271857:ENST00000368661:ENST00000624995 |
| 1:153779350:AG:A | | ENST00000271857:ENST00000368661:ENST00000624995 |
| 1:153779351:G:A | | ENST00000271857:ENST00000368661:ENST00000624995 |
| 1:153779352:G:A | | ENST00000271857:ENST00000368661:ENST00000624995 |
| 1:153779354:A:AG | | ENST00000271857:ENST00000368661:ENST00000624995 |
| 1:153779355:G:A | rs202142419 | ENST00000271857:ENST00000368661:ENST00000624995 |
| 1:153779355:G:A | rs202142419 | ENST00000271857:ENST00000368661:ENST00000624995 |
| 1:153779356:G:T | | ENST00000271857:ENST00000368661:ENST00000624995 |
| 1:153779357:G:T | | ENST00000271857:ENST00000368661:ENST00000624995 |
| 1:153779360:G:A | rs1420372320 | ENST00000271857:ENST00000368661:ENST00000624995 |
| 1:153779361:G:A | | ENST00000271857:ENST00000368661:ENST00000624995 |
| 1:153779361:G:T | | ENST00000271857:ENST00000368661:ENST00000624995 |
| 1:153779363:A:T | | ENST00000271857:ENST00000368661:ENST00000624995 |
| 1:153779364:T:C | rs1484559103 | ENST00000271857:ENST00000368661:ENST00000624995 |
| 1:153779366:G:A | rs777276444 | ENST00000271857:ENST00000368661:ENST00000624995 |
| 1:153779367:C:T | rs1472399917 | ENST00000271857:ENST00000368661:ENST00000624995 |
| 1:153779373:T:C | rs1378522119 | ENST00000271857:ENST00000368661:ENST00000624995 |
| 1:153779376:T:G | | ENST00000271857:ENST00000368661:ENST00000624995 |
| 1:153779379:T:G | | ENST00000271857:ENST00000368661:ENST00000624995 |
| 1:153779381:C:T | rs1375296136 | ENST00000271857:ENST00000368661:ENST00000624995 |
| 1:153779382:G:T | rs772337443 | ENST00000271857:ENST00000368661:ENST00000624995 |
| 1:153779382:G:A | rs772337443 | ENST00000271857:ENST00000368661:ENST00000624995 |
| 1:153779382:GT:G | rs1202063612 | ENST00000271857:ENST00000368661:ENST00000624995 |
| 1:153779383:T:TC | rs775999280 | ENST00000271857:ENST00000368661:ENST00000624995 |
| 1:153779383:TC:T | rs572408453 | ENST00000271857:ENST00000368661:ENST00000624995 |
| 1:153779384:C:A | rs775914465 | ENST00000271857:ENST00000368661:ENST00000624995 |
| 1:153779384:C:T | | ENST00000271857:ENST00000368661:ENST00000624995 |
| 1:153779385:C:G | rs1227264155 | ENST00000271857:ENST00000368661:ENST00000624995 |
| 1:153779385:C:A | | ENST00000271857:ENST00000368661:ENST00000624995 |
| 1:153779387:C:T | rs1001930434 | ENST00000271857:ENST00000368661:ENST00000624995 |
| 1:153779388:C:T | | ENST00000271857:ENST00000368661:ENST00000624995 |
| 1:153779388:C:G | rs764205164 | ENST00000271857:ENST00000368661:ENST00000624995 |
| 1:153779390:C:G | rs372347674 | ENST00000271857:ENST00000368661:ENST00000624995 |
| 1:153779390:C:A | rs372347674 | ENST00000271857:ENST00000368661:ENST00000624995 |
| 1:153779390:C:T | rs372347674 | ENST00000271857:ENST00000368661:ENST00000624995 |
| 1:153779391:A:C | | ENST00000271857:ENST00000368661:ENST00000624995 |
| 1:153779391:A:G | | ENST00000271857:ENST00000368661:ENST00000624995 |
| 1:153779392:C:A | | ENST00000271857:ENST00000368661:ENST00000624995 |
| 1:153779393:G:C | | ENST00000271857:ENST00000368661:ENST00000624995 |
| 1:153779393:G:T | rs111292643 | ENST00000271857:ENST00000368661:ENST00000624995 |
| 1:153779393:G:T | rs111292643 | ENST00000271857:ENST00000368661:ENST00000624995 |
| 1:153779393:G:A | rs111292643 | ENST00000271857:ENST00000368661:ENST00000624995 |
| 1:153779394:C:T | rs780806018 | ENST00000271857:ENST00000368661:ENST00000624995 |
| 1:153779398:G:C | | ENST00000271857:ENST00000368661:ENST00000624995 |
| 1:153779400:A:G | rs755765156 | ENST00000271857:ENST00000368661:ENST00000624995 |
| 1:153779400:A:AC | rs761005440 | ENST00000271857:ENST00000368661:ENST00000624995 |
| 1:153779402:C:G | | ENST00000271857:ENST00000368661:ENST00000624995 |
| 1:153779402:C:CT | | ENST00000271857:ENST00000368661:ENST00000624995 |
| 1:153779403:T:C | rs1042131900 | ENST00000271857:ENST00000368661:ENST00000624995 |
| 1:153779405:A:T | | ENST00000271857:ENST00000368661:ENST00000624995 |
| 1:153779406:T:C | rs777221507 | ENST00000271857:ENST00000368661:ENST00000624995 |
| 1:153779407:G:A | rs748849120 | ENST00000271857:ENST00000368661:ENST00000624995 |
| 1:153779412:T:TC | rs1179263691 | ENST00000271857:ENST00000368661:ENST00000624995 |
| 1:153779417:A:G | | ENST00000271857:ENST00000368661:ENST00000624995 |
| 1:153779418:C:T | | ENST00000271857:ENST00000368661:ENST00000624995 |
| 1:153779418:C:A | | ENST00000271857:ENST00000368661:ENST00000624995 |
| 1:153779420:C:T | | ENST00000271857:ENST00000368661:ENST00000624995 |
| 1:153779421:A:C | rs1178732071 | ENST00000271857:ENST00000368661:ENST00000624995 |
| 1:153779422:C:A | rs757753500 | ENST00000271857:ENST00000368661:ENST00000624995 |
| 1:153779423:G:T | | ENST00000271857:ENST00000368661:ENST00000624995 |
| 1:153779423:G:A | rs374422194 | ENST00000271857:ENST00000368661:ENST00000624995 |
| 1:153779429:G:A | | ENST00000271857:ENST00000368661:ENST00000624995 |
| 1:153779435:T:G | rs746234303 | ENST00000271857:ENST00000368661:ENST00000624995 |
| 1:153779437:GC:G | rs142189417;r | ENST00000271857:ENST00000368661:ENST00000624995 |
| | S560953426 | |
| 1:153779437:G:GC | | ENST00000271857:ENST00000368661:ENST00000624995 |
| 1:153779438:C:T | rs765169484 | ENST00000271857:ENST00000368661:ENST00000624995 |
| 1:153779440:A:AC | rs750087291 | ENST00000271857:ENST00000368661:ENST00000624995 |
| 1:153779441:C:T | rs148055664 | ENST00000271857:ENST00000368661:ENST00000624995 |
| 1:153779441:C:T | rs148055664 | ENST00000271857:ENST00000368661:ENST00000624995 |
| 1:153779442:C:T | rs11552969 | ENST00000271857:ENST00000368661:ENST00000624995 |
| 1:153779442:C:G | rs11552969 | ENST00000271857:ENST00000368661:ENST00000624995 |
| 1:153779442:CT:C | | ENST00000271857:ENST00000368661:ENST00000624995 |
| 1:153779444:T:A | rs112342423 | ENST00000271857:ENST00000368661:ENST00000624995 |
| 1:153779445:A:G | | ENST00000271857:ENST00000368661:ENST00000624995 |
| 1:153779446:T:G | | ENST00000271857:ENST00000368661:ENST00000624995 |
| 1:153779447:G:T | rs1301783644 | ENST00000271857:ENST00000368661:ENST00000624995 |
| 1:153779450:C:G | | ENST00000271857:ENST00000368661:ENST00000624995 |
| 1:153779450:C:T | rs150607273 | ENST00000271857:ENST00000368661:ENST00000624995 |
| 1:153779450:C:T | rs150607273 | ENST00000271857:ENST00000368661:ENST00000624995 |
| 1:153779451:G:A | rs200824960 | ENST00000271857:ENST00000368661:ENST00000624995 |
| 1:153779451:G:T | | ENST00000271857:ENST00000368661:ENST00000624995 |
| 1:153779451:G:C | | ENST00000271857:ENST00000368661:ENST00000624995 |
| 1:153779451:G:A | rs200824960 | ENST00000271857:ENST00000368661:ENST00000624995 |
| 1:153779452:G:GT | rs1393517739 | ENST00000271857:ENST00000368661:ENST00000624995 |
| 1:153779453:C:G | rs766334578 | ENST00000271857:ENST00000368661:ENST00000624995 |
| 1:153779453:C:T | rs766334578 | ENST00000271857:ENST00000368661:ENST00000624995 |
| 1:153779454:C:T | rs1459215175 | ENST00000271857:ENST00000368661:ENST00000624995 |
| 1:153779456:C:T | rs143069452 | ENST00000271857:ENST00000368661:ENST00000624995 |
| 1:153779457:G:C | rs373742297 | ENST00000271857:ENST00000368661:ENST00000624995 |
| 1:153779457:G:A | rs373742297 | ENST00000271857:ENST00000368661:ENST00000624995 |
| 1:153779459:T:G | rs1193406424 | ENST00000271857:ENST00000368661:ENST00000624995 |
| 1:153779460:T:C | | ENST00000271857:ENST00000368661:ENST00000624995 |
| 1:153779461:C:G | | ENST00000271857:ENST00000368661:ENST00000624995 |
| 1:153779463:T:G | | ENST00000271857:ENST00000368661:ENST00000624995 |
| 1:153779465:A:T | | ENST00000271857:ENST00000368661:ENST00000624995 |
| 1:153779467:G:C | rs367786493 | ENST00000271857:ENST00000368661:ENST00000624995 |
| 1:153779468:C:T | | ENST00000271857:ENST00000368661:ENST00000624995 |
| 1:153779468:C:G | | ENST00000271857:ENST00000368661:ENST00000624995 |
| 1:153779469:T:C | | ENST00000271857:ENST00000368661:ENST00000624995 |
| 1:153779469:TC:T | rs1428285810 | ENST00000271857:ENST00000368661:ENST00000624995 |
| 1:153779472:A:G | rs755744516 | ENST00000271857:ENST00000368661:ENST00000624995 |
| 1:153779474:G:A | | ENST00000271857:ENST00000368661:ENST00000624995 |
| 1:153779475:T:C | | ENST00000271857:ENST00000368661:ENST00000624995 |
| 1:153779823:C:CA | | ENST00000271857:ENST00000368661:ENST00000624995 |
| 1:153779825:G:A | rs940220692 | ENST00000271857:ENST00000368661:ENST00000624995 |
| 1:153779828:G:T | | ENST00000271857:ENST00000368661:ENST00000624995 |
| 1:153779830:CTT:C | | ENST00000271857:ENST00000368661:ENST00000624995 |
| 1:153779832:T:A | | ENST00000271857:ENST00000368661:ENST00000624995 |
| 1:153779835:G:A | | ENST00000271857:ENST00000368661:ENST00000624995 |
| 1:153779841:A:G | rs764612952 | ENST00000271857:ENST00000368661:ENST00000624995 |
| 1:153779846:G:C | rs1041309320 | ENST00000271857:ENST00000368661:ENST00000624995 |
| 1:153779848:C:A | | ENST00000271857:ENST00000368661:ENST00000624995 |
| 1:153779848:C:G | rs199542091 | ENST00000271857:ENST00000368661:ENST00000624995 |
| 1:153779851:T:TCA | rs754569929 | ENST00000271857:ENST00000368661:ENST00000624995 |
| AA | | |
| 1:153779854:A:C | | ENST00000271857:ENST00000368661:ENST00000624995 |
| 1:153779856:C:G | rs559054034 | ENST00000271857:ENST00000368661:ENST00000624995 |
| 1:153779856:C:A | rs559054034 | ENST00000271857:ENST00000368661:ENST00000624995 |
| 1:153779857:A:G | rs377177127 | ENST00000271857:ENST00000368661:ENST00000624995 |
| 1:153779859:C:G | rs748248699 | ENST00000271857:ENST00000368661:ENST00000624995 |
| 1:153779859:C:T | rs748248699 | ENST00000271857:ENST00000368661:ENST00000624995 |
| 1:153779861:G:C | | ENST00000271857:ENST00000368661:ENST00000624995 |
| 1:153779865:G:A | | ENST00000271857:ENST00000368661:ENST00000624995 |
| 1:153779868:C:T | rs749428513 | ENST00000271857:ENST00000368661:ENST00000624995 |
| 1:153779869:G:A | rs191183550 | ENST00000271857:ENST00000368661:ENST00000624995 |
| 1:153779871:A:G | | ENST00000271857:ENST00000368661:ENST00000624995 |
| 1:153779872:T:C | | ENST00000271857:ENST00000368661:ENST00000624995 |
| 1:153779875:C:G | rs775467690 | ENST00000271857:ENST00000368661:ENST00000624995 |
| 1:153779880:G:A | | ENST00000271857:ENST00000368661:ENST00000624995 |
| 1:153779883:G:A | | ENST00000271857:ENST00000368661:ENST00000624995 |
| 1:153779884:G:C | rs768373838 | ENST00000271857:ENST00000368661:ENST00000624995 |
| 1:153779889:G:A | rs201498802 | ENST00000271857:ENST00000368661:ENST00000624995 |
| 1:153779889:G:A | rs201498802 | ENST00000271857:ENST00000368661:ENST00000624995 |
| 1:153779890:A:G | | ENST00000271857:ENST00000368661:ENST00000624995 |
| 1:153779892:C:CCT | | ENST00000271857:ENST00000368661:ENST00000624995 |
| GGGGT | | |
| 1:153779892:C:A | | ENST00000271857:ENST00000368661:ENST00000624995 |
| 1:153779892:C:G | rs1160368100 | ENST00000271857:ENST00000368661:ENST00000624995 |
| 1:153779895:A:G | | ENST00000271857:ENST00000368661:ENST00000624995 |
| 1:153779898:A:G | rs1361004914 | ENST00000271857:ENST00000368661:ENST00000624995 |
| 1:153779899:C:A | | ENST00000271857:ENST00000368661:ENST00000624995 |
| 1:153779901:C:G | | ENST00000271857:ENST00000368661:ENST00000624995 |
| 1:153779902:T:C | | ENST00000271857:ENST00000368661:ENST00000624995 |
| 1:153779904:T:C | | ENST00000271857:ENST00000368661:ENST00000624995 |
| 1:153779907:G:A | | ENST00000271857:ENST00000368661:ENST00000624995 |
| 1:153779908:AC:A | | ENST00000271857:ENST00000368661:ENST00000624995 |
| 1:153779911:C:A | rs1290510824 | ENST00000271857:ENST00000368661:ENST00000624995 |
| 1:153779917:A:G | rs1348345866 | ENST00000271857:ENST00000368661:ENST00000624995 |
| 1:153779919:G:A | rs145546950 | ENST00000271857:ENST00000368661:ENST00000624995 |
| 1:153779931:G:A | | ENST00000271857:ENST00000368661:ENST00000624995 |
| 1:153779934:G:A | rs142522769 | ENST00000271857:ENST00000368661:ENST00000624995 |
| 1:153779938:G:T | rs1280744792 | ENST00000271857:ENST00000368661:ENST00000624995 |
| 1:153779938:G:GT | | ENST00000271857:ENST00000368661:ENST00000624995 |
| GCCTACC | | |
| 1:153779938:G:A | | ENST00000271857:ENST00000368661:ENST00000624995 |
| 1:153779940:G:A | rs1406253728 | ENST00000271857:ENST00000368661:ENST00000624995 |
| 1:153779941:C:G | | ENST00000271857:ENST00000368661:ENST00000624995 |
| 1:153779941:C:T | | ENST00000271857:ENST00000368661:ENST00000624995 |
| 1:153779943:T:C | | ENST00000271857:ENST00000368661:ENST00000624995 |
| 1:153779944:A:G | | ENST00000271857:ENST00000368661:ENST00000624995 |
| 1:153779949:C:T | | ENST00000271857:ENST00000368661:ENST00000624995 |
| 1:153779950:C:A | | ENST00000271857:ENST00000368661:ENST00000624995 |
| 1:153779950:C:T | | ENST00000271857:ENST00000368661:ENST00000624995 |
| 1:153779953:T:C | | ENST00000271857:ENST00000368661:ENST00000624995 |
| 1:153779953:TCA:T | rs781223890 | ENST00000271857:ENST00000368661:ENST00000624995 |
| 1:153779954:C:CA | | ENST00000271857:ENST00000368661:ENST00000624995 |
| 1:153779955:A:G | | ENST00000271857:ENST00000368661:ENST00000624995 |
| 1:153779956:C:A | | ENST00000271857:ENST00000368661:ENST00000624995 |
| 1:153779956:C:T | | ENST00000271857:ENST00000368661:ENST00000624995 |
| 1:153779961:G:A | rs1399303095 | ENST00000271857:ENST00000368661:ENST00000624995 |
| 1:153779962:C:G | | ENST00000271857:ENST00000368661:ENST00000624995 |
| 1:153779962:C:T | rs1237094484 | ENST00000271857:ENST00000368661:ENST00000624995 |
| 1:153779964:C:T | rs1437271974 | ENST00000271857:ENST00000368661:ENST00000624995 |
| 1:153779965:G:C | | ENST00000271857:ENST00000368661:ENST00000624995 |
| 1:153779965:G:A | rs140906306 | ENST00000271857:ENST00000368661:ENST00000624995 |
| 1:153779965:G:A | rs140906306 | ENST00000271857:ENST00000368661:ENST00000624995 |
| 1:153779969:C:G | | ENST00000271857:ENST00000368661:ENST00000624995 |
| 1:153779970:A:G | | ENST00000271857:ENST00000368661:ENST00000624995 |
| 1:153779971:G:A | rs1426967802 | ENST00000271857:ENST00000368661:ENST00000624995 |
| 1:153779973:G:A | rs35517522 | ENST00000271857:ENST00000368661:ENST00000624995 |
| 1:153779973:G:A | rs35517522 | ENST00000271857:ENST00000368661:ENST00000624995 |
| 1:153779974:C:T | rs367682870 | ENST00000271857:ENST00000368661:ENST00000624995 |
| 1:153779976:C:A | rs368098884 | ENST00000271857:ENST00000368661:ENST00000624995 |
| 1:153779977:T:C | | ENST00000271857:ENST00000368661:ENST00000624995 |
| 1:153779982:G:T | rs776270134 | ENST00000271857:ENST00000368661:ENST00000624995 |
| 1:153779985:G:A | rs1277417928 | ENST00000271857:ENST00000368661:ENST00000624995 |
| 1:153779988:A:C | rs761710758 | ENST00000271857:ENST00000368661:ENST00000624995 |
| 1:153779989:A:G | | ENST00000271857:ENST00000368661:ENST00000624995 |
| 1:153779990:C:A | rs769324071 | ENST00000271857:ENST00000368661:ENST00000624995 |
| 1:153779991:C:G | | ENST00000271857:ENST00000368661:ENST00000624995 |
| 1:153779992:T:G | | ENST00000271857:ENST00000368661:ENST00000624995 |
| 1:153779994:C:T | rs1347564125 | ENST00000271857:ENST00000368661:ENST00000624995 |
| 1:153779995:G:C | | ENST00000271857:ENST00000368661:ENST00000624995 |
| 1:153779995:G:A | rs201820055 | ENST00000271857:ENST00000368661:ENST00000624995 |
| 1:153779995:G:A | rs201820055 | ENST00000271857:ENST00000368661:ENST00000624995 |
| 1:153779997:A:G | | ENST00000271857:ENST00000368661:ENST00000624995 |
| 1:153780000:T:C | | ENST00000271857:ENST00000368661:ENST00000624995 |
| 1:153780002:A:G | rs199682747 | ENST00000271857:ENST00000368661:ENST00000624995 |
| 1:153780002:A:G | rs199682747 | ENST00000271857:ENST00000368661:ENST00000624995 |

In some embodiments, the subject's aggregate burden of having any one or more SLC27A3 variant nucleic acid molecules encoding an SLC27A3 predicted loss-of-function polypeptide represents a weighted sum of a plurality of any of the SLC27A3 variant nucleic acid molecules encoding an SLC27A3 predicted loss-of-function polypeptide. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the SLC27A3 gene where the genetic burden is the number of alleles multiplied by the association estimate with Asthma or related outcome for each allele (e.g., a weighted polygenic burden score). This can include any genetic variants, regardless of their genomic annotation, in proximity to the SLC27A3 gene (up to 10 Mb around the gene) that show a non-zero association with asthma-related traits in a genetic association analysis. In some embodiments, when the subject has an aggregate burden above a desired threshold score, the subject has a decreased risk of developing asthma. In some embodiments, when the subject has an aggregate burden below a desired threshold score, the subject has an increased risk of developing asthma.

In some embodiments, the aggregate burden may be divided into quintiles, e.g., top quintile, intermediate quintile, and bottom quintile, wherein the top quintile of aggregate burden corresponds to the lowest risk group and the bottom quintile of aggregate burden corresponds to the highest risk group. In some embodiments, a subject having a greater aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with asthma in the top 10%, top 20%, top 30%, top 40%, or top 50% of p-value range for the association. In some embodiments, each of the identified genetic variants comprise the genetic variants having association with asthma with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with asthma with p-value of less than 5×10⁻⁸. In some embodiments, the identified genetic variants comprise genetic variants having association with asthma in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having aggregate burdens in the bottom decile, quintile, or tertile in a reference population. The threshold of the aggregate burden is determined on the basis of the nature of the intended practical application and the risk difference that would be considered meaningful for that practical application.

In some embodiments, when a subject is identified as having an increased risk of developing asthma, the subject is further administered a therapeutic agent that treats or prevents asthma, and/or an SLC27A3 inhibitor, as described herein. For example, when the subject is SLC27A3 reference, and therefore has an increased risk of developing asthma, the subject is administered an SLC27A3 inhibitor. In some embodiments, such a subject is also administered a therapeutic agent that treats or prevents asthma. In some embodiments, when the subject is heterozygous for an SLC27A3 variant nucleic acid molecule encoding an SLC27A3 predicted loss-of-function polypeptide, the subject is administered the therapeutic agent that treats or prevents asthma in a dosage amount that is the same as or less than a standard dosage amount, and is also administered an SLC27A3 inhibitor. In some embodiments, the subject is SLC27A3 reference. In some embodiments, the subject is heterozygous for an SLC27A3 variant nucleic acid molecule encoding an SLC27A3 predicted loss-of-function polypeptide. Furthermore, when the subject has a lower aggregate burden for having an SLC27A3 variant nucleic acid molecule encoding an SLC27A3 predicted loss-of-function polypeptide, and therefore has an increased risk of developing asthma, the subject is administered a therapeutic agent that treats or prevents asthma. In some embodiments, when the subject has a lower aggregate burden for having an SLC27A3 variant nucleic acid molecule encoding an SLC27A3 predicted loss-of-function polypeptide, the subject is administered the therapeutic agent that treats or prevents asthma in a dosage amount that is the same as or greater than the standard dosage amount administered to a subject who has a greater aggregate burden for having an SLC27A3 variant nucleic acid molecule encoding an SLC27A3 predicted loss-of-function polypeptide.

The present disclosure also provides methods of detecting the presence or absence of an SLC27A3 variant nucleic acid molecule (i.e., a genomic nucleic acid molecule, an mRNA molecule, or a cDNA molecule produced from an mRNA molecule) encoding an SLC27A3 predicted loss-of-function polypeptide in a biological sample from a subject. It is understood that gene sequences within a population and mRNA molecules encoded by such genes can vary due to polymorphisms such as single-nucleotide polymorphisms. The sequences provided herein for the SLC27A3 variant genomic nucleic acid molecule, SLC27A3 variant mRNA molecule, and SLC27A3 variant cDNA molecule are only exemplary sequences. Other sequences for the SLC27A3 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any SLC27A3 variant nucleic acid molecule encoding an SLC27A3 predicted loss-of-function polypeptide, preliminary processing designed to isolate or enrich the biological sample for the genomic DNA can be employed. A variety of techniques may be used for this purpose. When detecting the level of any SLC27A3 variant mRNA molecule, different techniques can be used enrich the biological sample with mRNA molecules. Various methods to detect the presence or level of an mRNA molecule or the presence of a particular variant genomic DNA locus can be used.

In some embodiments, detecting an SLC27A3 variant nucleic acid molecule encoding an SLC27A3 predicted loss-of-function polypeptide in a subject comprises performing a sequence analysis on a biological sample obtained from the subject to determine whether an SLC27A3 genomic nucleic acid molecule in the biological sample, and/or an SLC27A3 mRNA molecule in the biological sample, and/or an SLC27A3 cDNA molecule produced from an mRNA molecule in the biological sample, comprises one or more variations that cause a loss-of-function (partial or complete) or are predicted to cause a loss-of-function (partial or complete).

In some embodiments, the methods of detecting the presence or absence of an SLC27A3 variant nucleic acid molecule encoding an SLC27A3 predicted loss-of-function polypeptide (such as, for example, a genomic nucleic acid molecule, an mRNA molecule, and/or a cDNA molecule produced from an mRNA molecule) in a subject, comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an SLC27A3 genomic nucleic acid molecule or mRNA molecule, and if mRNA, optionally reverse transcribing the mRNA into cDNA. Such assays can comprise, for example determining the identity of these positions of the particular SLC27A3 nucleic acid molecule. In some embodiments, the method is an in vitro method.

In some embodiments, the determining step, detecting step, or sequence analysis comprises sequencing at least a portion of the nucleotide sequence of the SLC27A3 genomic nucleic acid molecule, the SLC27A3 mRNA molecule, or the SLC27A3 cDNA molecule in the biological sample, wherein the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an SLC27A3 genomic nucleic acid molecule is analyzed. In some embodiments, only an SLC27A3 mRNA is analyzed. In some embodiments, only an SLC27A3 cDNA obtained from SLC27A3 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an SLC27A3 variant genomic sequence, variant mRNA sequence, or variant cDNA sequence and not the corresponding SLC27A3 reference sequence under stringent conditions, and determining whether hybridization has occurred.

In some embodiments, the determining step, detecting step, or sequence analysis comprises: a) amplifying at least a portion of the nucleic acid molecule that encodes the SLC27A3 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an SLC27A3 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to SLC27A3 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein, and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to SLC27A3 variant genomic nucleic acid molecules, SLC27A3 variant mRNA molecules, and/or SLC27A3 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the SLC27A3 variant missense genomic nucleic acid molecules, SLC27A3 variant mRNA molecules, and/or SLC27A3 variant cDNA molecules disclosed herein. The primers described herein can be used to amplify SLC27A3 variant genomic nucleic acid molecules, SLC27A3 variant mRNA molecules, or SLC27A3 variant cDNA molecules, or a fragment thereof.

In the context of the disclosure “specifically hybridizes” means that the probe or primer (such as, for example, the alteration-specific probe or alteration-specific primer) does not hybridize to a nucleic acid sequence encoding an SLC27A3 reference genomic nucleic acid molecule, an SLC27A3 reference mRNA molecule, and/or an SLC27A3 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleotide sequence of an SLC27A3 reference genomic nucleic acid molecule is set forth in SEQ ID NO:1 (ENSG00000143554.14 encompassing chr1:153,774,354-153,780,157 in the GRCh38/hg38 human genome assembly).

The nucleotide sequence of an SLC27A3 reference mRNA molecule is set forth in SEQ ID NO:2. The nucleotide sequence of another SLC27A3 reference mRNA molecule is set forth in SEQ ID NO:3. The nucleotide sequence of another SLC27A3 reference mRNA molecule is set forth in SEQ ID NO:4. The nucleotide sequence of another SLC27A3 reference mRNA molecule is set forth in SEQ ID NO:5. The nucleotide sequence of another SLC27A3 reference mRNA molecule is set forth in SEQ ID NO:6. The nucleotide sequence of another SLC27A3 reference mRNA molecule is set forth in SEQ ID NO:7. The nucleotide sequence of another SLC27A3 reference mRNA molecule is set forth in SEQ ID NO:8. The nucleotide sequence of another SLC27A3 reference mRNA molecule is set forth in SEQ ID NO:9. The nucleotide sequence of another SLC27A3 reference mRNA molecule is set forth in SEQ ID NO:10. The nucleotide sequence of another SLC27A3 reference mRNA molecule is set forth in SEQ ID NO:11. The nucleotide sequence of another SLC27A3 reference mRNA molecule is set forth in SEQ ID NO:12. The nucleotide sequence of another SLC27A3 reference mRNA molecule is set forth in SEQ ID NO:13. The nucleotide sequence of another SLC27A3 reference mRNA molecule is set forth in SEQ ID NO:14.

The nucleotide sequence of an SLC27A3 reference cDNA molecule is set forth in SEQ ID NO:15. The nucleotide sequence of another SLC27A3 reference cDNA molecule is set forth in SEQ ID NO:16. The nucleotide sequence of another SLC27A3 reference cDNA molecule is set forth in SEQ ID NO:17. The nucleotide sequence of another SLC27A3 reference cDNA molecule is set forth in SEQ ID NO:18. The nucleotide sequence of another SLC27A3 reference cDNA molecule is set forth in SEQ ID NO:19. The nucleotide sequence of another SLC27A3 reference cDNA molecule is set forth in SEQ ID NO:20. The nucleotide sequence of another SLC27A3 reference cDNA molecule is set forth in SEQ ID NO:21. The nucleotide sequence of another SLC27A3 reference cDNA molecule is set forth in SEQ ID NO:22. The nucleotide sequence of another SLC27A3 reference cDNA molecule is set forth in SEQ ID NO:23. The nucleotide sequence of another SLC27A3 reference cDNA molecule is set forth in SEQ ID NO:24. The nucleotide sequence of another SLC27A3 reference cDNA molecule is set forth in SEQ ID NO:25. The nucleotide sequence of another SLC27A3 reference cDNA molecule is set forth in SEQ ID NO:26. The nucleotide sequence of another SLC27A3 reference cDNA molecule is set forth in SEQ ID NO:27.

The amino acid sequence of an SLC27A3 reference polypeptide is set forth in SEQ ID NO:28, and is 811 amino acids in length. The amino acid sequence of another SLC27A3 reference polypeptide is set forth in SEQ ID NO:29, and is 683 amino acids in length. The amino acid sequence of another SLC27A3 reference polypeptide is set forth in SEQ ID NO:30, and is 648 amino acids in length. The amino acid sequence of another SLC27A3 reference polypeptide is set forth in SEQ ID NO:31, and is 640 amino acids in length. The amino acid sequence of another SLC27A3 reference polypeptide is set forth in SEQ ID NO:32, and is 730 amino acids in length. The amino acid sequence of another SLC27A3 reference polypeptide is set forth in SEQ ID NO:33, and is 288 amino acids in length. The amino acid sequence of another SLC27A3 reference polypeptide is set forth in SEQ ID NO:34, and is 700 amino acids in length. The amino acid sequence of another SLC27A3 reference polypeptide is set forth in SEQ ID NO:35, and is 399 amino acids in length. The amino acid sequence of another SLC27A3 reference polypeptide is set forth in SEQ ID NO:36, and is 152 amino acids in length. The amino acid sequence of another SLC27A3 reference polypeptide is set forth in SEQ ID NO:37, and is 144 amino acids in length. The amino acid sequence of another SLC27A3 reference polypeptide is set forth in SEQ ID NO:38, and is 229 amino acids in length.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

The present disclosure also provides vectors comprising any one or more of the nucleic acid molecules disclosed herein. In some embodiments, the vectors comprise any one or more of the nucleic acid molecules disclosed herein and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides therapeutic agents that treat or prevent asthma for use in the treatment or prevention of asthma in a subject having: an SLC27A3 variant genomic nucleic acid molecule encoding an SLC27A3 predicted loss-of-function polypeptide; an SLC27A3 variant mRNA molecule encoding an SLC27A3 predicted loss-of-function polypeptide; or an SLC27A3 variant cDNA molecule encoding an SLC27A3 predicted loss-of-function polypeptide. Any of the therapeutic agents that treat or prevent asthma described herein can be used in these methods. The asthma can be allergic asthma, nonallergic asthma, exercise-induced bronchoconstriction, ACOS, eosinophilic asthma, childhood asthma, and/or occupational asthma.

The present disclosure also provides uses of therapeutic agents that treat or prevent asthma for use in the preparation of a medicament for treating or preventing asthma in a subject having: an SLC27A3 variant genomic nucleic acid molecule encoding an SLC27A3 predicted loss-of-function polypeptide; an SLC27A3 variant mRNA molecule encoding an SLC27A3 predicted loss-of-function polypeptide; or an SLC27A3 variant cDNA molecule encoding an SLC27A3 predicted loss-of-function polypeptide. Any of the therapeutic agents that treat or prevent asthma described herein can be used in these methods. The asthma can be allergic asthma, nonallergic asthma, exercise-induced bronchoconstriction, ACOS, eosinophilic asthma, childhood asthma, and/or occupational asthma.

The present disclosure also provides SLC27A3 inhibitors for use in the treatment or prevention of asthma in a subject that: a) is reference for an SLC27A3 genomic nucleic acid molecule, an SLC27A3 mRNA molecule, or an SLC27A3 cDNA molecule; or b) is heterozygous for: i) an SLC27A3 variant genomic nucleic acid molecule encoding an SLC27A3 predicted loss-of-function polypeptide; ii) an SLC27A3 variant mRNA molecule encoding an SLC27A3 predicted loss-of-function polypeptide; or iii) an SLC27A3 variant cDNA molecule encoding an SLC27A3 predicted loss-of-function polypeptide. Any of the SLC27A3 inhibitors described herein can be used in these methods. The asthma can be allergic asthma, nonallergic asthma, exercise-induced bronchoconstriction, ACOS, eosinophilic asthma, childhood asthma, and/or occupational asthma.

The present disclosure also provides uses of SLC27A3 inhibitors in the preparation of a medicament for treating or preventing asthma in a subject that: a) is reference for an SLC27A3 genomic nucleic acid molecule, an SLC27A3 mRNA molecule, or an SLC27A3 cDNA molecule; or b) is heterozygous for: i) an SLC27A3 variant genomic nucleic acid molecule encoding an SLC27A3 predicted loss-of-function polypeptide; ii) an SLC27A3 variant mRNA molecule encoding an SLC27A3 predicted loss-of-function polypeptide; or iii) an SLC27A3 variant cDNA molecule encoding an SLC27A3 predicted loss-of-function polypeptide. Any of the SLC27A3 inhibitors described herein can be used in these methods. The asthma can be allergic asthma, nonallergic asthma, exercise-induced bronchoconstriction, ACOS, eosinophilic asthma, childhood asthma, and/or occupational asthma.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Burden of Rare pLOFs and Deleterious Variants in SLC27A3 Associated with Lower Risk of Childhood Asthma

The exomes of 454,787 UKB study participants were sequenced, with 95.8% of targeted bases covered at a depth of 20× or greater, as previously described (Szustakowski, Advancing Human Genetics Research and Drug Discovery through Exome Sequencing of the UK Biobank. bioRxiv, 2021; and Van Hout et al., Nature, 2020). Twelve million variants were identified in 39 million base pairs across the coding regions of 18,659 genes (data not shown), which exceeds by about 1.3-fold the coding variation contained in the combined TOPMed3 and gnomAD4 datasets (9.5 million variants), and by about 8-fold the coding variation accessible in the UKB study through imputation (1.6 million variants). Among the variants identified were 3,375,252 (median of 10,260 per individual) synonymous U.S. Pat. No. 7,689,495 (9,284 per individual) missense and 889,957 (212 per individual) putative loss-of-function (pLOF) variants (data not shown), of which about half were observed only once in this dataset (singleton variants; data not shown).

A novel association between lower risk of childhood asthma and a burden of rare pLOFs and deleterious variants was discovered in SLC27A3 (3,787 carriers; OR=0.65, 95% Cl 0.55 to 0.76, P=8.2×10⁻⁸). SLC27A3 encodes an acyl-CoA synthetase that activates long-chain fatty acids, is most highly expressed in artery, adipose and lung tissue, and is up-regulated in lung cancer. The association with asthma was supported by the following additional observations. First, a burden of rare pLOF and deleterious variants was also associated with lower blood eosinophil counts (5,227 carriers; effect=−0.045 SD units, 95% Cl −0.070 to −0.020, P=4.4×10⁻⁴), a cell type with critical effector functions in allergic asthma. Second, there were consistent protective associations in the GHS cohort with both asthma (1,354 carriers; OR=0.87, 95% Cl 0.754 to 1.006, P=0.060) and eosinophil counts (1,755 carriers; effect=−0.052 SD units, 95% Cl −0.096 to −0.008, P=0.021). These results suggest that SLC27A3 harbors both loss of function variants, associated with protection from and higher risk of asthma.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having asthma, exercise-induced bronchoconstriction, or asthma-COPD overlap syndrome (ACOS) or at risk of developing asthma, exercise-induced bronchoconstriction, or ACOS, the method comprising administering a Solute Carrier Family 27 Member 3 (SLC27A3) inhibitor to the subject.

2. The method according to claim 1, wherein the is allergic asthma, nonallergic asthma, eosinophilic asthma, childhood asthma, or occupational asthma.

3-8. (canceled)

9. The method according to claim 1, wherein the SLC27A3 inhibitor comprises an inhibitory nucleic acid molecule that hybridizes to an SLC27A3 nucleic acid molecule.

10. The method according to claim 9, wherein the inhibitory nucleic acid molecule comprises an antisense nucleic acid molecule, a small interfering RNA (siRNA), or a short hairpin RNA (shRNA).

11-16. (canceled)

17. The method according to claim 1, further comprising detecting the presence or absence of an SLC27A3 variant nucleic acid molecule encoding an SLC27A3 predicted loss-of-function polypeptide in a biological sample from the subject.

18. The method according to claim 17, further comprising administering a therapeutic agent that treats, prevents, or inhibits asthma, exercise-induced bronchoconstriction, or ACOS in a standard dosage amount to a subject wherein the SLC27A3 variant nucleic acid molecule is absent from the biological sample.

19. The method according to claim 17, further comprising administering a therapeutic agent that treats, prevents, or inhibits asthma, exercise-induced bronchoconstriction, or ACOS in a dosage amount that is the same as or less than a standard dosage amount to a subject that is heterozygous for the SLC27A3 variant nucleic acid molecule.

20. The method according to claim 17, wherein the SLC27A3 predicted variant nucleic acid molecule is a splice-site variant, a stop-gain variant, a start-loss variant, a stop-loss variant, a frameshift variant, or an in-frame indel variant, or a variant that encodes a truncated SLC27A3 predicted loss-of-function polypeptide.

21. The method according to claim 20, wherein the SLC27A3 variant nucleic acid molecule encodes a truncated SLC27A3 predicted loss-of-function polypeptide.

22. A method of treating a subject with a therapeutic agent that treats or prevents asthma, exercise-induced bronchoconstriction, or asthma-COPD overlap syndrome (ACOS), wherein the subject has asthma, exercise-induced bronchoconstriction, or asthma-COPD overlap syndrome (ACOS) or is at risk of developing asthma, exercise-induced bronchoconstriction, or ACOS, the method comprising the steps of:
determining whether the subject has a Solute Carrier Family 27 Member 3 (SLC27A3) variant nucleic acid molecule encoding an SLC27A3 predicted loss-of-function polypeptide by:
obtaining or having obtained a biological sample from the subject; and
performing or having performed a sequence analysis on the biological sample to determine if the subject has a genotype comprising the SLC27A3 variant nucleic acid molecule; and
administering or continuing to administer the therapeutic agent that treats or prevents asthma, exercise-induced bronchoconstriction, or ACOS in a standard dosage amount to a subject that is SLC27A3 reference, and/or administering an SLC27A3 inhibitor to the subject;
administering or continuing to administer the therapeutic agent that treats or prevents asthma, exercise-induced bronchoconstriction, or ACOS in an amount that is the same as or less than a standard dosage amount to a subject that is heterozygous for the SLC27A3 variant nucleic acid molecule, and/or administering an SLC27A3 inhibitor to the subject; or
administering or continuing to administer the therapeutic agent that treats or prevents asthma, exercise-induced bronchoconstriction, or ACOS in an amount that is the same as or less than a standard dosage amount to a subject that is homozygous for the SLC27A3 variant nucleic acid molecule;
wherein the presence of a genotype having the SLC27A3 variant nucleic acid molecule encoding the SLC27A3 predicted loss-of-function polypeptide indicates the subject has a decreased risk of developing asthma, exercise-induced bronchoconstriction, or ACOS.

23. The method according to claim 22, wherein the subject is SLC27A3 reference, and the subject is administered or continued to be administered the therapeutic agent that treats, prevents, or inhibits asthma, exercise-induced bronchoconstriction, or ACOS in a standard dosage amount, and is administered an SLC27A3 inhibitor.

24. The method according to claim 22, wherein the subject is heterozygous for an SLC27A3 variant nucleic acid molecule, and the subject is administered or continued to be administered the therapeutic agent that treats, prevents, or inhibits asthma, exercise-induced bronchoconstriction, or ACOS in an amount that is the same as or less than a standard dosage amount, and is administered an SLC27A3 inhibitor.

25. The method according to claim 22, wherein the SLC27A3 variant nucleic acid molecule is a splice-site variant, a stop-gain variant, a start-loss variant, a stop-loss variant, a frameshift variant, or an in-frame indel variant, or a variant that encodes a truncated SLC27A3 predicted loss-of-function polypeptide.

26. The method according to claim 22, wherein the SLC27A3 variant nucleic acid molecule encodes a truncated SLC27A3 predicted loss-of-function polypeptide.

27. The method according to claim 22, wherein the SLC27A3 inhibitor comprises an inhibitory nucleic acid molecule that hybridizes to an SLC27A3 nucleic acid molecule.

28. The method according to claim 27, wherein the inhibitory nucleic acid molecule comprises an antisense nucleic acid molecule, a small interfering RNA (siRNA), or a short hairpin RNA (shRNA).

29-34. (canceled)

35. The method according to claim 22, wherein the asthma is allergic asthma, nonallergic asthma, eosinophilic asthma, childhood asthma, or occupational asthma.

36-41. (canceled)

42. The method according to claim 22, wherein the asthma is allergic asthma, and the therapeutic agent is chosen from inhaled steroids, anticholinergic maintenance medications, leukotriene modifiers, and biologic immunomodulators, or any combination thereof.

43. The method according to claim 22, wherein the asthma is nonallergic asthma, and therapeutic agent is chosen from inhaled steroids, anticholinergic maintenance medications, leukotriene modifiers, and biologic immunomodulators, or any combination thereof.

44. The method according to claim 22, wherein exercise-induced bronchoconstriction is treated, and the therapeutic agent is chosen from inhaled steroids, anticholinergic maintenance medications, leukotriene modifiers, and biologic immunomodulators, or any combination thereof.

45. The method according to claim 22, wherein ACOS is treated, and the therapeutic agent is chosen from inhaled steroids, anticholinergic maintenance medications, leukotriene modifiers, and biologic immunomodulators, or any combination thereof.

46. The method according to claim 22, wherein the asthma is eosinophilic asthma, and the therapeutic agent is chosen from inhaled steroids, anticholinergic maintenance medications, leukotriene modifiers, and biologic immunomodulators, or any combination thereof.

47. The method according to claim 22, wherein the asthma is childhood asthma, and the therapeutic agent is chosen from inhaled steroids, anticholinergic maintenance medications, leukotriene modifiers, and biologic immunomodulators, or any combination thereof.

48. The method according to claim 22, wherein the asthma is occupational asthma, and the therapeutic agent is chosen from inhaled steroids, anticholinergic maintenance medications, leukotriene modifiers, and biologic immunomodulators, or any combination thereof.

49-113. (canceled)
